(19) United States
(12) Patent Application Publication
(10) Pub. No.: US 2026/0257987 A1
(43) Pub. Date: Sep. 3, 2026

(54) COMPOUNDS AND METHOD FOR UPREGULATION OF P53 THROUGH INDUCTION OF MDM2 DEGRADATION

(71) Applicant: Georgia State University Research Foundation, Inc., Atlanta, GA (US)

(72) Inventors: Binghe Wang, Marietta, GA (US); Xiaoxiao Yang, Marietta, GA (US); Ravi Tripathi, Atlanta, GA (US)

(21) Appl. No.: 18/877,722

(22) PCT Filed: Jun. 20, 2023
(86) PCT No.: PCT/US2023/068720

§ 371 Date: Dec. 20, 2024

Related U.S. Application Data
(60) Provisional application No. 63/353,783, filed on Jun. 20, 2022.

(51) Int. Cl. C07C 233/33; A61K 31/167; A61K 31/18; A61P 35/02; C07C 311/11

(52) U.S. Cl. CPC C07C 233/33; A61K 31/167; A61K 31/18; A61P 35/02; C07C 311/11; C07C 2603/24

(57) ABSTRACT

Anthraquinone analogs that have anticancer properties are disclosed. Pharmaceutical formulations in forms suitable for the delivery of the compounds to a subject in need thereof are disclosed. Methods of using the compounds for treating or ameliorating one or more symptoms associated with a cancer, such as leukemia, in a subject are also disclosed. The methods include (i) administering to the subject the pharmaceutical formulation containing one or more compounds, for one or more times. The compounds can induce apoptosis in cancer cells, such as leukemia cells harboring WT-p53 (e.g., EU-1 leukemia cells), MCF7 cells, RS4;11 cells, and/or HeLa cells, by downregulating MDM2 and upregulating p53 in these cancer cells.

### CROSS-REFERENCE TO RELATED APPLICATIONS

This application claims the benefit of and priority to U.S. Ser. No. 63/353,783, filed Jun. 20, 2022, which is specifically incorporated by reference herein in its entirety.

### FIELD OF THE INVENTION

This invention is generally in the field of anthraquinone analogs and methods of uses thereof.

### BACKGROUND OF THE INVENTION

Acute lymphocytic leukemia (ALL) is the most prevalent cancer among children and adolescents worldwide. ALL cells are immature malignant lymphoblasts with unsuppressed proliferation ability. If untreated, ALL normally leads to death within a few months. One well-characterized genomic factor is the ubiquitously adoption of a malfunctioned p53/MDM2 signaling pathway. While p53 is a tumor suppressor, controlling apoptosis and thus serving as a brake in cellular growth and replication, MDM2 is an E3 ligase that targets p53 to proteolysis. In normal cells, there is a well-maintained feedback control of the p53-MDM2 axis. However, in cancerous cells, the function of p53 is commonly inactivated by either mutation to the TP53 gene or through overexpression of MDM2, leading to the progression of cancer. In some leukemia phenotypes, both p53 mutation and MDM2 overexpression can be found, which correlates with their aggressive malignancy. In ALL patients, about 30% have over-expression of MDM2, leading to unrestrained proliferation. From a therapeutic point of view, overexpression of MDM2 has also been implicated in chemoresistance and relapse after treatment.

Restoring p53 function through intercepting the p53-MDM2 axis by inducing MDM2 degradation or inhibiting its E3 ligase activity is an effective therapeutic approach for ALL. For example, nutlins, MK-8242, RG711225, and some stapled peptides bind to the p53 binding domain of MDM2 and thereby prevent ubiquitination of p53. A small molecule Nilotinib downregulates MDM2 by promoting its self-ubiquitination. Another small molecule, triptolide inhibits mRNA expression of MDM2 in cancer cells. PROTAC molecules that target p53-MDM2 have been reported to downregulate MDM2 through recruiting other E3 ligase or MDM2 itself to induce polyubiquitination of MDM2 and the subsequent proteasomal degradation. Further, a series of anthraquinones with anticancer activity was discovered, such as BW-AQ-101 (2-chloro-N-(4,5-dihydroxy-9,10-dioxo-9,10-dihydroanthracen-2-yl)acetamide) that showeded an IC₅₀ of about 0.8 μM in EU-1 ALL cells (an in-house WT-p53 ALL cell line separated from a pediatric ALL patients).

There remains a need to develop anthraquinone analogs that process improved potency against cancer cells, such as EU-1 ALL cells.

Therefore, it is the object of the present invention to provide anthraquinone analogs that possess potency against cancer cells.

It is a further object of the present invention to provide anthraquinone analogs that possess improved potency against cancer cells.

It is a further object of the present invention to provide methods of using anthraquinone analogs as anticancer agents.

### SUMMARY OF THE INVENTION

Synthetic anthraquinone analogs (also referred herein as “compounds”) that possess anticancer properties and their methods of using have been developed. In particular, the compounds disclosed herein are suitable for treating or ameliorating symptom(s) of leukemia, such as Acute lymphocytic leukemia (ALL). Without being bound to any theory, the anthraquinone core structure and modifications at the R¹/R¹′ and/or R² positions of the anthraquinone core contribute to the antiancer activity of these compounds, rendering them useful in inducing apoptosis of cancer cells. For example, the compounds disclosed herein can downregulate MDM2 and upregulate p53 in cancer cells harboring WT-p53, such as leukemia cells harboring WT-p53 (e.g., EU-1 leukemia cells), MCF7 cells, RS4;11 cells, and HeLa cells. Additionally, the compounds disclosed herein can down-regulate MDM2 without the general cytotoxicity by inducing DNA damage and do not cardiotoxicity at the effective dosage in animal models as usually seen with doxorubicin and mitoxantrone.

The compounds can have the structures of Formula I:

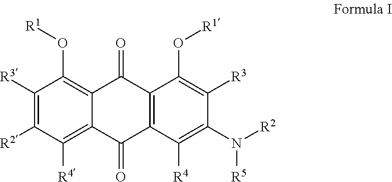

wherein: (a) R¹ and R¹′ can be independently a hydrogen, a hydroxyalkyl, or a carbonyl (e.g., a ketone), wherein at least one of R¹ and R¹′ is not hydrogen; (b) R² can be an electrophilic group; (c) R⁵ can be a hydrogen, a substituted or unsubstituted alkyl, a substituted or unsubstituted alkenyl, or a substituted or unsubstituted alkynyl; (d) R³, R⁴, R²′, R³′, and R⁴′ can be independently a hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclyl, a halogen, a hydroxyl, an amino, an amido, an azo, an alkoxy, a polyether, a thiol, a cyano, a nitro, or a carbonyl; (e) each substituent can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an azido, an oxo, a silyl, a sulfinyl, a sulfonyl, a sulfonic acid, a phosphonium, a phosphanyl, a phosphoryl, or a phosphonyl.

In some forms of Formula I, R² can be a carbonyl, a sulfinyl, a sulfonyl, or a sulfamoyl.

In some forms of Formula I, R² can be

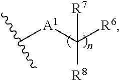

and wherein A¹ can be

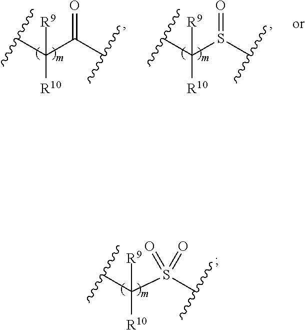

n and m can be independently an integer from 0 to 6, from 0 to 5, from 0 to 4, from 0 to 3, or from 0 to 2, such as 1 or 0; R⁶ can be a halogen (e.g., fluorine, chlorine, bromine, or iodine), a cyano, an azido, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, or a substituted or unsubstituted heterocyclyl; and R⁷—R¹⁰ can be independently a hydrogen, a substituted or unsubstituted alkyl, a substituted or unsubstituted alkenyl, a substituted or unsubstituted alkynyl, a halogen, a hydroxyl, an amino, an amido, an alkoxy, an azo, an azido, a polyether, a thiol, a cyano, a nitro, a nitrile, a carbonyl, a sulfinyl, a sulfonyl, or a sulfamoyl.

In some forms of Formula I, R² can be

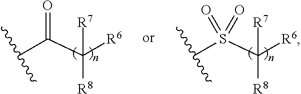

and wherein: n can be an integer from 0 to 3, from 0 to 2, 0 or 1; R⁶ can be a halogen (e.g., fluorine, chlorine, bromine, or iodine), a cyano, an azido, a substituted or unsubstituted alkenyl, or a substituted or unsubstituted alkynyl; and R⁷ and R⁸ can be independently a hydrogen, a substituted or unsubstituted alkyl, a substituted or unsubstituted alkenyl, a substituted or unsubstituted alkynyl, a halogen, a cyano, or an azido.

In some forms, R⁶ can be a halogen (e.g., fluorine, chlorine, bromine, or iodine), an azido, an unsubstituted C₂-C₆ alkenyl, or an unsubstituted C₂-C₆ alkynyl, such as a chlorine, an azido, an unsubstituted ethylenyl, an unsubstituted propylenyl, an unsubstituted ethynyl, or an unsubstituted propynyl. In some forms, R⁶ can be fluorine, bromine, iodine, an azido, an unsubstituted C₃-C₆ alkenyl, or an unsubstituted C₂-C₆ alkynyl.

In some forms of Formula I, R¹ and R¹′ can be independently a hydrogen,

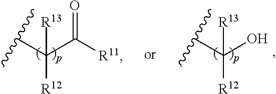

wherein at least one of R¹ and R¹′ is not hydrogen, and wherein: p can be an integer from 0 to 6, from 0 to 5, from 0 to 4, from 1 to 5, from 2 to 5, from 1 to 4, or from 2 to 4, such as 3; and R¹¹ R¹³ can be independently a hydrogen, a substituted or unsubstituted alkyl, a substituted or unsubstituted alkenyl, a substituted or unsubstituted alkynyl, a halogen, a hydroxyl, an amino, an amido, an alkoxy, an azo, an azido, a polyether, a thiol, a cyano, a nitro, or a nitrile.

In some forms, R¹¹ can be a hydrogen, a hydroxyl, or a substituted or unsubstituted alkyl; and R¹² and R¹³ can be independently a hydrogen or a substituted or unsubstituted alkyl.

In some forms, R¹¹ can be an unsubstituted C₁-C₆ alkyl, an unsubstituted C₁-C₅ alkyl, an unsubstituted C₁-C₄ alkyl, an unsubstituted C₁-C₃ alkyl, an unsubstituted methyl or ethyl, such as methyl.

In some forms, R¹² and R¹³ can be hydrogen.

In some forms, R⁵ can be hydrogen.

In some forms of Formula I, R³, R⁴, R²′, R³′, and R⁴′ can be independently a hydrogen or a substituted or unsubstituted alkyl.

In some forms of Formula I, R³, R⁴, R²′, R³′, and R⁴′ can be hydrogen.

In some forms, the compounds disclosed herein can have the structure of:

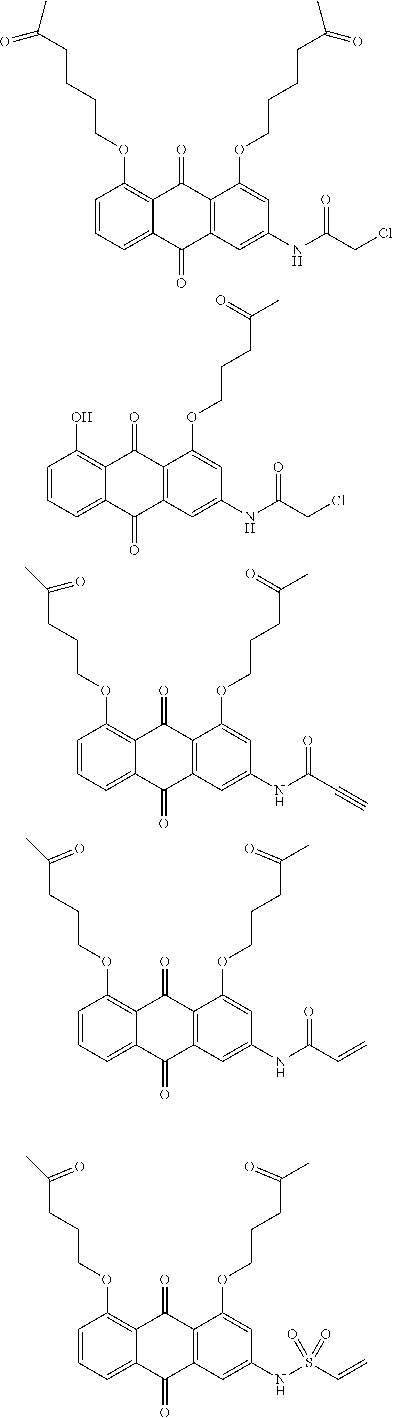

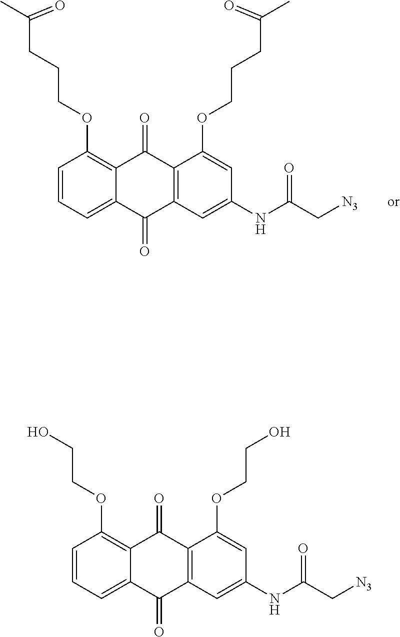 [SMILES: CC(=O)CCCOc1cccc2c1C(=O)c1c(OCCCC(C)=O)cc(NC(=O)CN=[N+]=[N-])cc1C2=O ; [N-]=[N+]=NCC(=O)Nc1cc(OCCO)c2c(c1)C(=O)c1cccc(OCCO)c1C2=O]

In some forms, the compound is not

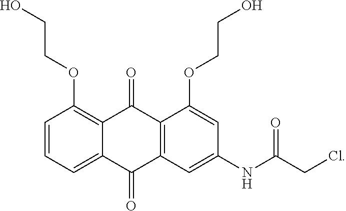 [SMILES: O=C(CCl)Nc1cc(OCCO)c2c(c1)C(=O)c1cccc(OCCO)c1C2=O]

In some forms, the compound is not

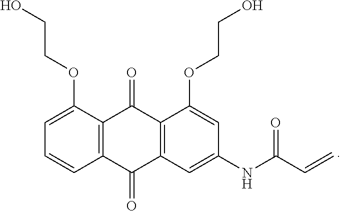 [SMILES: C=CC(=O)Nc1cc(OCCO)c2c(c1)C(=O)c1cccc(OCCO)c1C2=O]

In some forms, the compound is not

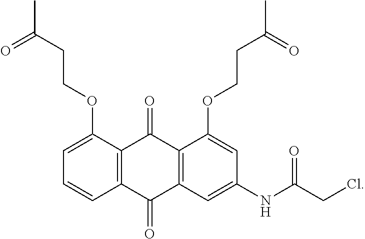 [SMILES: CC(=O)CCOc1cccc2c1C(=O)c1c(OCCC(C)=O)cc(NC(=O)CCl)cc1C2=O]

In some forms, the compound is not

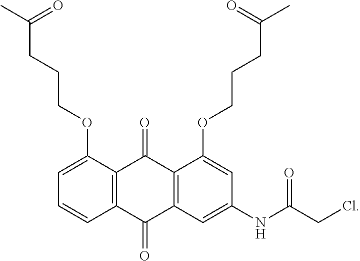 [SMILES: CC(=O)CCCOc1cccc2c1C(=O)c1c(OCCCC(C)=O)cc(NC(=O)CCl)cc1C2=O]

In some forms, the compound is not

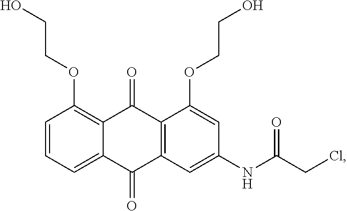 [SMILES: O=C(CCl)Nc1cc(OCCO)c2c(c1)C(=O)c1cccc(OCCO)c1C2=O]

not

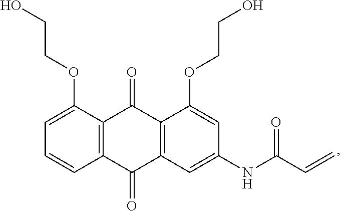 [SMILES: C=CC(=O)Nc1cc(OCCO)c2c(c1)C(=O)c1cccc(OCCO)c1C2=O]

not

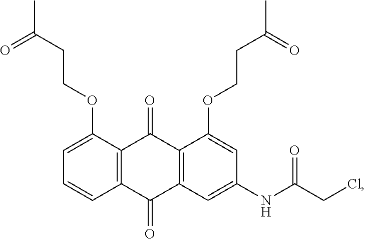 [SMILES: CC(=O)CCOc1cccc2c1C(=O)c1c(OCCC(C)=O)cc(NC(=O)CCl)cc1C2=O]

and not

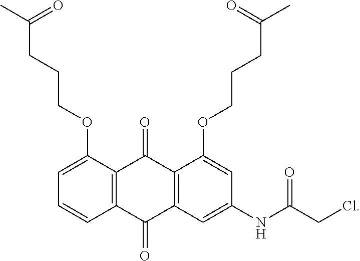 [SMILES: CC(=O)CCCOc1cccc2c1C(=O)c1c(OCCCC(C)=O)cc(NC(=O)CCl)cc1C2=O]

In some forms, the compound can have an IC₅₀ value against test cancer cells comparable or lower than an IC₅₀ value of BW-AQ-101 and/or BW-AQ-238 (structure shown below) against the same test cancer cells, tested under the same conditions. The test cancer cells can be cancer cells that harbor WT-p53, such as leukemia cells harboring WT-p53 (e.g., EU-1 leukemia cells), MCF7 cells, RS4;11 cells, and/or HeLa cells.

Pharmaceutical formulations containing one or more compounds disclosed herein; and a pharmaceutically acceptable carrier and/or excipient are disclosed. In some forms, the total concentration of the one or more compounds in the pharmaceutical formulation can be at least 0.001 wt %, at least 0.005 wt %, at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.1 wt % to 20 wt %, from 0.01 wt % to 10 wt %, from 0.05 wt % to 10 wt %, or from 0.1 wt % to 10 wt %.

In some forms, the one or more compounds in the pharmaceutical formulation can be in an effective amount to treat or ameliorate one or more symptoms associated with a cancer in a subject. In some forms, the one or more compounds in the pharmaceutical formulation can be in an effective amount to induce apoptosis of cancer cells in a subject, such as cancer cells harboring WT-p53 (e.g., leukemia cells harboring WT-p53, MCF7 cells, RS4;11 cells, or HeLa cells, or a combination thereof). In some forms, the one or more compounds in the pharmaceutical formulation can be in an effective amount to downregulate MDM2 and upregulate p53 in these cancer cells compared to the cancer cells before treatment as shown by Western blot.

The pharmaceutical formulation may contain a second active agent, optionally more than one second active agent, such as one or more anticancer agents.

Methods for treating or ameliorating one or more symptoms associated with a cancer in a subject in need thereof are also disclosed. Generally, the method includes (i) administering to the subject the pharmaceutical formulation described herein, wherein step (i) occurs one or more times. The method may further include administering to the subject a second active agent, such as an anticancer agent, prior to, during, and/or subsequent to step (i). In some forms, the cancer being treated can be leukemia, such as Acute lymphocytic leukemia (ALL), Acute myelogenous leukemia (AML), Chronic lymphocytic leukemia (CLL), or Chronic myelogenous leukemia (CML).

The method may include only a single administration of the pharmaceutical formulation or more than one step of administering to the subject the pharmaceutical formulation, such as by oral administration, parenteral administration, inhalation, mucosal administration, topical or a combination thereof.

In some forms, following the single administration or multiple administrations of the pharmaceutical formulation, an effective amount of the compounds to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment, is administered to the subject. The effective amount of the compounds administered to the subject can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 20 mg/kg.

When multiple administration steps are performed in the disclosed method, the administration can be performed every day, every 2 days, every 3 days, every 5 days, every 7 days, every 10 days, every two weeks, or every month for a period from one day to 6 months, from one day to 5 months, from one day to 4 months, from one day to 3 months, from one day to 2 months, from one day to thirty days, or from one day to ten days.

Methods for treating cancer cells in a subject in need thereof are also disclosed. The cancer cells can be cancer cells harboring WT-p53, for example, leukemia cells harboring WT-p53 (e.g., EU-1 leukemia cells), MCF7 cells, RS4;11 cells, and/or HeLa cells. Generally, the method includes (i) administering to the subject the pharmaceutical formulation, wherein step (i) occurs one or more times. The method can include only a single administration of the pharmaceutical formulation or more than one step of administering to the subject the pharmaceutical formulation. Typically, following the single or multiple administration steps, an effective amount of the compounds to induce apoptosis of the cancer cells is administered to the subject.

In some forms, the effective amount of the compounds administered to the subject is effective to downregulate MDM2 and upregulate p53 in test cancer cells compared to the test cancer cells before treatment as shown by Western blot. In some forms, the compound(s) administered to the subject can have an IC₅₀ value against test cancer cells comparable to or lower than an IC₅₀ value of BW-AQ-101 and/or BW-AQ-238 against the same test cancer cells, tested under the same conditions.

### BRIEF DESCRIPTION OF THE DRAWINGS

FIGS. 1A-1B are graphs showing detailed correlation for structure determination of BW-AQ-336 by HMBC 2D-NMR. FIG. 1C is a graph showing HMBC 2D-NMR of BW-AQ-336.

FIGS. 2A-2B are Western blot graphs showing the downregulation of MDM2 and upregulation p53 by BW-AQ-295 in dosage-(FIG. 2A) and time-dependent (FIG. 2B) manner in EU-1 leukemia cells. GAPDH was probed as the loading control.

FIGS. 2C-2D are bar graphs showing the relative quantification of Western blot of EU-1 cells treated with BW-AQ-295: dosage dependency at 6 h (FIG. 2C); and time dependency at the dosage of 0.8 μM (FIG. 2D). Data are shown as the fold change compared to the control group (dosage=0 or time=0) after normalization by GAPDH. * p<0.05, ** p<0.01, versus vehicle group.

FIG. 3 is a graph showing the time plot of NAC-reactivity of the anthraquinone analogs at 37° C. in PBS.

FIGS. 4A-4B are Western blot graphs showing the downregulation of MDM2 and upregulation p53 by BW-AQ-350 in dosage-(FIG. 4A) and time-dependent (FIG. 4B) manner in EU-1 leukemia cells. GAPDH was probed as the loading control. FIGS. 4C-4D are bar graphs showing the relative quantification of Western blot of EU-1 cells treated with BW-AQ-350: dosage dependency at 5 h (FIG. 4C); and time dependency at the dosage of 1 μM (FIG. 4D). Data are shown as the fold change compared to the control group (dosage=0 or time=0) after normalization by GAPDH. * p<0.05, ** p<0.01, versus vehicle group.

FIGS. 5A-5D are graphs showing in-vitro cytotoxicity results of BW-AQ-365. FIG. 5A shows the cell viability of EU-1 cells at 24 hours vs. concentration of BW-AQ-365. FIG. 5B shows the cell viability of RS4;11 cells (with a cell density of 10000 cells/well) at 48 hours vs. concentration of BW-AQ-365. FIG. 5C shows the cell viability of RS4;11 cells (with a cell density of 25000 cells/well) at 48 hours vs. concentration of BW-AQ-365. FIG. 5D shows the cell viability of RS4;11 cells (with a cell density of 25000 cells/well) at 72 hours vs. concentration of BW-AQ-365. FIG. 5E is a graph showing the cell viability of RS4;11 cells (with a cell density of 25000 cells/well) at 48 hours vs. concentration of Doxorubicin.

### DETAILED DESCRIPTION OF THE INVENTION

### I. Definitions

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

“Substituted alkyl” refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF₃, —CH₂—CF₃, —CCl₃); —CN; —NCOCOCH₂CH₂; —NCOCOCHCH; and —NCS; and combinations thereof.

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C=C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkynyl,” as used herein, refers to straight or branched chain, or cyclic carbon-containing alkynyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkynyl group” is a cycloalkynyl group where at least one of the carbon atoms of the ring is substituted with a heteroatom such as, but not limited to, nitrogen, oxygen, sulphur, or phosphorus.

“Aryl,” as used herein, refers to C₅-C₂₆-membered aromatic or fused aromatic ring systems. Examples of aromatic groups are benzene, naphthalene, anthracene, phenanthrene, chrysene, pyrene, corannulene, coronene, etc.

The term “substituted aryl” refers to an aryl group, wherein one or more hydrogen atoms on one or more aromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₂₆-membered aromatic or fused aromatic ring systems, in which one or more carbon atoms on one or more aromatic ring structures have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “substituted heteroaryl” refers to a heteroaryl group in which one or more hydrogen atoms on one or more heteroaromatic rings are substituted with one or more substituents including, but not limited to, halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, imino, alkylthio, sulfate, sulfonate, sulfamoyl, sulfoxide, sulfonamido, sulfonyl, heterocyclyl, alkylaryl, haloalkyl (such as CF₃, —CH₂—CF₃, —CCl₃), —CN, aryl, heteroaryl, and combinations thereof.

The term “polyaryl” refers to a chemical moiety that includes two or more fused aryl groups. When two or more fused heteroaryl groups are involved, the chemical moiety can be referred to as a “polyheteroaryl.”

The term “substituted polyaryl” refers to a polyaryl in which one or more of the aryls are substituted, with one or more substituents including, but not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (or quarternized amino), amido, amidine, imine, cyano, nitro, azido, sulfhydryl, alkylthio, sulfate, sulfonate, sulfamoyl, sulfoxide, sulfonamido, sulfonyl, heterocyclyl, alkylaryl, haloalkyl, —CN, aryl, heteroaryl, and combinations thereof. When a polyheteroaryl is involved, the chemical moiety can be referred to as a “substituted polyheteroaryl.”

The term “cyclic ring” or “cyclic group” refers to a substituted or unsubstituted monocyclic ring or a substituted or unsubstituted polycyclic ring (such as those formed from single or fused ring systems), such as a substituted or unsubstituted cycloalkyl, a substituted or unsubstituted cycloalkenyl, a substituted or unsubstituted cycloalkynyl, or a substituted or unsubstituted heterocyclyl, that have from three to 30 carbon atoms, as geometric constraints permit. The substituted cycloalkyls, cycloalkenyls, cycloalkynyls, and heterocyclyls are substituted as defined above for the alkyls, alkenyls, alkynyls, and heterocyclyls, respectively.

The term “aralkyl” as used herein is an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” or “alkoxy,” “aroxy” or “aryloxy,” generally describe compounds represented by the formula —ORY, wherein R^{v} includes, but is not limited to, a substituted or unsubstituted alkyl, a substituted or unsubstituted alkenyl, a substituted or unsubstituted alkynyl, a substituted or unsubstituted cycloalkyl, a substituted or unsubstituted heterocyclyl, a substituted or unsubstituted cycloalkenyl, a substituted or unsubstituted heterocycloalkenyl, a substituted or unsubstituted aryl, a substituted or unsubstituted heteroaryl, a substituted or unsubstituted polyaryl, a substituted or unsubstituted polyheteroaryl, a substituted or unsubstituted arylalkyl, a substituted or unsubstituted heteroalkyl, a substituted or unsubstituted alkylaryl, a substituted or unsubstituted alkylheteroaryl, a substituted or unsubstituted aralkyl, a substituted or unsubstituted carbonyl, a phosphonium, a phosphanyl, a phosphonyl, a sulfinyl, a silyl, a thiol, an amido, and an amino. Exemplary alkoxyl groups include methoxy, ethoxy, propyloxy, tert-butoxy and the like. A “lower alkoxy” group is an alkoxy group containing from one to six carbon atoms. An “ether” is two functional groups covalently linked by an oxygen as defined below. Accordingly, the substituent of an alkyl that renders that alkyl an ether is or resembles an alkoxyl, such as can be represented by one of—O-alkyl, —O-alkenyl, —O-alkynyl, —O-arakyl, —O-aryl, —O-heteroaryl, —O-polyaryl, —O-polyheteroaryl, —O-heterocyclyl, etc.

The term “substituted alkoxy” refers to an alkoxy group having one or more substituents replacing one or more hydrogen atoms on one or more carbons of the alkoxy backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, oxo, sulfate, sulfonate, sulfamoyl, sulfonamido, sulfonyl, heterocyclyl, alkylaryl, haloalkyl, —CN, aryl, heteroaryl, and combinations thereof.

The term “ether” as used herein is represented by the formula A²OA¹, where A² and A¹ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a phosphonyl, a sulfinyl, a silyl, a thiol, a substituted or unsubstituted carbonyl, an alkoxy, an amido, or an amino, described above.

The term “polyether” as used herein is represented by the formula:

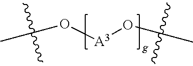

where A³ can be, independently, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl, a substituted or unsubstituted polyaryl, a substituted or unsubstituted polyheteroaryl, a phosphonium, a phosphanyl, a substituted or unsubstituted carbonyl, an alkoxy, an amido, or an amino, described above; g can be a positive integer from 1 to 30.

The term “phenoxy” is art recognized and refers to a compound of the formula —ORY wherein R^{Y} is C₆H₅ (i.e., —O—C₆H₅). One of skill in the art recognizes that a phenoxy is a species of the aroxy genus.

The term “substituted phenoxy” refers to a phenoxy group, as defined above, having one or more substituents replacing one or more hydrogen atoms on one or more carbons of the phenyl ring. Such substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof.

The terms “aroxy” and “aryloxy,” as used interchangeably herein, are represented by—O-aryl or —O-heteroaryl, wherein aryl and heteroaryl are as defined herein.

The terms “substituted aroxy” and “substituted aryloxy,” as used interchangeably herein, represent-O-aryl or —O-heteroaryl, having one or more substituents replacing one or more hydrogen atoms on one or more ring atoms of the aryl and heteroaryl, as defined herein. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “amino” as used herein includes the group

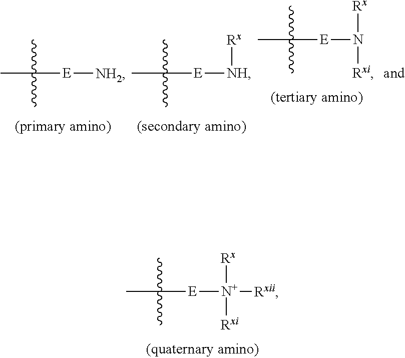

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The terms “amide” or “amido” are used interchangeably, refer to both “unsubstituted amido” and “substituted amido” and are represented by the general formula:

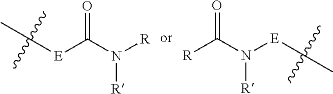

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R″, or R and R′ taken together with the N atom to which they are attached complete a heterocycle having from 3 to 14 atoms in the ring structure; R″ ″′ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed. It is understood by those of ordinary skill in the art, that the E groups listed above are divalent (e.g., methylene, ethane-1,2-diyl, ethene-1,2-diyl, 1,4-phenylene, cyclohexane-1,2-diyl).

“Carbonyl,” as used herein, is art-recognized and includes such moieties as can be represented by the general formula:

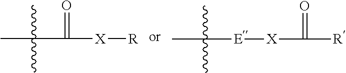

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or (CH₂)ₘ—R″, or a pharmaceutical acceptable salt; E″ is absent, or E″ is a substituted or unsubstituted alkyl, a substituted or unsubstituted alkenyl, a substituted or unsubstituted alkynyl, a substituted or unsubstituted aralkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. It is understood by those of ordinary skill in the art, that the E″ groups listed above are divalent (e.g., methylene, ethane-1,2-diyl, ethene-1,2-diyl, 1,4-phenylene, cyclohexane-1,2-diyl). Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “phosphanyl” is represented by the formula

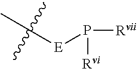

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi} and R^{vii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R″, or R^{vi} and R^{vii} taken together with the P atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonium” is represented by the formula

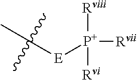

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted cycloalkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R^{vi}, R^{vii}, and R^{viii} each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R″, or R^{vi}, R^{vii}, and R^{viii} taken together with the P⁺ atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphonyl” is represented by the formula

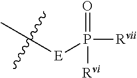

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, oxygen, alkoxy, aroxy, or substituted alkoxy or substituted aroxy, wherein, independently of E, R^{vi} and R^{vi} are independently a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R^{vi} and R^{vii} taken together with the P atom to which they are attached complete a heterocycle having from 3 to 14 atoms in the ring structure; R″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “phosphoryl” defines a phosphonyl in which E is absent, oxygen, alkoxy, aroxy, substituted alkoxy or substituted aroxy, as defined above, and independently of E, R^{vi} and R^{vi} are independently hydroxyl, alkoxy, aroxy, substituted alkoxy or substituted aroxy, as defined above. When E is oxygen, the phosphoryl cannot be attached to another chemical species, such as to form an oxygen-oxygen bond, or other unstable bonds, as understood by one of ordinary skill in the art. When E, R^{vi} and R^{vii} are substituted, the substituents include, but are not limited to, halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfinyl” is represented by the formula

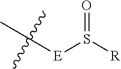

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a silyl, a thiol, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonyl” is represented by the formula

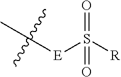

wherein E is absent, or E is a substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted arylalkyl, etc.), a substituted or unsubstituted aryl, a substituted or unsubstituted heteroaryl, a substituted or unsubstituted heterocyclyl, a substituted or unsubstituted polyaryl, a substituted or unsubstituted polyheteroaryl, wherein independently of E, R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R′″, or E and R taken together with the S atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonic acid” refers to a sulfonyl, as defined above, wherein R is hydroxyl, and E is absent, or E is substituted or unsubstituted cycloalkyl, substituted or unsubstituted heterocyclyl, substituted or unsubstituted alkylaryl, substituted or unsubstituted arylalkyl, substituted or unsubstituted aryl, a substituted or unsubstituted polyaryl, a substituted or unsubstituted polyheteroaryl, or substituted or unsubstituted heteroaryl. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfate” refers to a sulfonyl, as defined above, wherein E is absent, oxygen, alkoxy, aroxy, substituted alkoxy or substituted aroxy, as defined above, and R is independently hydroxyl, alkoxy, aroxy, substituted alkoxy or substituted aroxy, as defined above. When E is oxygen, the sulfate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfonate” refers to a sulfonyl, as defined above, wherein E is oxygen, alkoxy, aroxy, substituted alkoxy or substituted aroxy, as defined above, and R is independently hydrogen, substituted or unsubstituted alkyl, substituted or unsubstituted alkenyl, substituted or unsubstituted alkynyl, substituted or unsubstituted amino, substituted or unsubstituted cycloalkyl, substituted or unsubstituted heterocyclyl, substituted or unsubstituted aralkyl, substituted or unsubstituted alkylaryl, substituted or unsubstituted arylalkyl, substituted or unsubstituted aryl, substituted or unsubstituted heteroaryl, a substituted or unsubstituted polyaryl, a substituted or unsubstituted polyheteroaryl, —(CH₂)ₘ—R″, R′″ represents a hydroxy group, substituted or unsubstituted carbonyl group, an aryl, a cycloalkyl ring, a cycloalkenyl ring, a heterocycle, an amido, an amino, or a polycycle; and m is zero or an integer ranging from 1 to 8. When E is oxygen, sulfonate cannot be attached to another chemical species, such as to form an oxygen-oxygen bond, or other unstable bonds, as understood by one of ordinary skill in the art. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “sulfamoyl” refers to a sulfonamide or sulfonamide represented by the formula

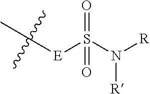

wherein E is absent, or E is substituted or unsubstituted alkyl, a substituted or unsubstituted alkenyl, a substituted or unsubstituted alkynyl, a substituted or unsubstituted aralkyl (e.g., a substituted or unsubstituted alkylaryl, a substituted or unsubstituted cycloalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl, etc.), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″ ″, or R and R′ taken together with the N atom to which they are attached complete a heterocycle having from 3 to 14 atoms in the ring structure; R“ ” represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The term “silyl group” as used herein is represented by the formula —SiRR′R,″ where R, R′, and R″ can be, independently, a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, a phosphonyl, a sulfinyl, a thiol, an amido, an amino, an alkoxy, or an oxo, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted, independently, with the substituents described above in the definition of “substituted.”

The numerical ranges disclose individually each possible number that such a range could reasonably encompass, as well as any sub-ranges and combinations of sub-ranges encompassed therein. For example, in a given range carbon range of C₃-C₉, the range also discloses C₃, C₄, C₅, C₆, C₇, C₈, and Co, as well as any subrange between these numbers (for example, C₄-C₆), and any possible combination of ranges possible between these values. In yet another example, a given temperature range may be from about 25° C. to 30° C., where the range also discloses temperatures that can be selected independently from about 25, 26, 27, 28, 29, and 30° C., as well as any range between these numbers (for example, 26 to 28° C.), and any possible combination of ranges between these values.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

“Analog” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered an analog of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified, if it induces the same pharmacological effect, physiological effect, or both, as the specified compound. “Analog” can also refer to a modification including, but not limited to, hydrolysis, reduction, or oxidation products, of the disclosed compounds. Hydrolysis, reduction, and oxidation reactions are known in the art.

### II. Compositions

Synthetic anthraquinone analogs (also referred herein as “compounds”) have been developed. The synthetic compounds disclosed herein have anticancer properties and should be suitable for use in the treatment or amelioration of the symptoms of multiple types of cancers. In particular, these compounds are suitable for treating or ameliorating symptom(s) of leukemia, such as Acute lymphocytic leukemia (ALL). Without being bound to any theory, the anthraquinone core structure and modifications at the R¹/R¹′ and/or R² positions of the anthraquinone core contribute to the antiancer activity of these compounds, rendering them useful in inducing apoptosis of cancer cells. For example, the compounds disclosed herein can downregulate MDM2 and upregulate p53 in cancer cells harboring WT-p53, such as leukemia cells harboring WT-p53 (e.g., EU-1 leukemia cells), MCF7 cells, RS4;11 cells, and HeLa cells. Additionally, the compounds disclosed herein can down-regulate MDM2 without the general cytotoxicity by inducing DNA damage and do not cardiotoxicity at the effective dosage in animal models as usually seen with doxorubicin and mitoxantrone.

In some forms, The compounds disclosed herein have an IC₅₀ value against test cancer cells comparable or lower than an IC₅₀ value of BW-AQ-101 and/or BW-AQ-238 against the same test cancer cells, tested under the same conditions. Generally, if the IC₅₀ value of the compounds against the test cancer cells is +10% of the IC₅₀ value of BW-AQ-101 or BW-AQ-238 against the same test cancer cells tested under the same conditions, then the two IC₅₀ values are considered comparable. The term “same conditions” means test is performed using the same protocol, such as same amount of cells and enzymes, same dye and dye concentration, same inbubation time and temperature, etc.

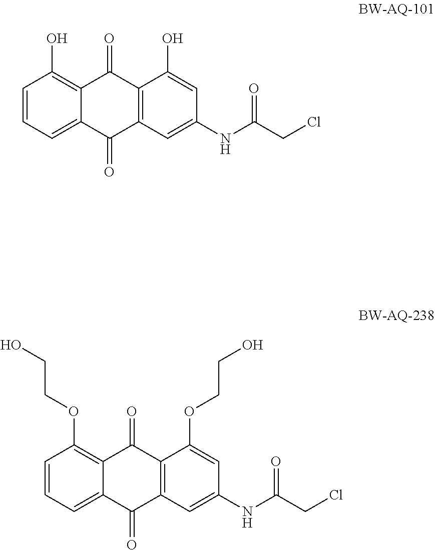 [SMILES: O=C(CCl)Nc1cc(O)c2c(c1)C(=O)c1cccc(O)c1C2=O ; O=C(CCl)Nc1cc(OCCO)c2c(c1)C(=O)c1cccc(OCCO)c1C2=O]

Pharmaceutical formulations containing the compounds are also disclosed.

### A. Compounds

### 1. Compound Structures

The compounds can have the structures of Formula I:

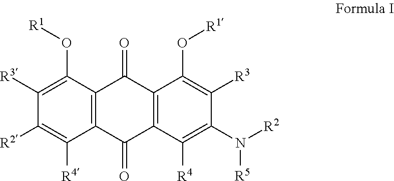

wherein: (a) R¹ and R¹′ can be independently a hydrogen, a hydroxyalkyl, or a carbonyl (e.g., a ketone), wherein at least one of R¹ and R¹′ is not hydrogen; (b) R² can be an electrophilic group; (c) R⁵ can be a hydrogen, a substituted or unsubstituted alkyl, a substituted or unsubstituted alkenyl, or a substituted or unsubstituted alkynyl; (d) R³, R⁴, R²′, R³′, and R⁴′ can be independently a hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclyl, a halogen, a hydroxyl, an amino, an amido, an azo, an alkoxy, a polyether, a thiol, a cyano, a nitro, or a carbonyl; (e) each substituent can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an azido, an oxo, a silyl, a sulfinyl, a sulfonyl, a sulfonic acid, a phosphonium, a phosphanyl, a phosphoryl, or a phosphonyl.

In some forms of Formula I, R² can be a carbonyl, a sulfinyl, a sulfonyl, or a sulfamoyl.

In some forms of Formula I, R² can be

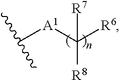

and wherein A¹ can be

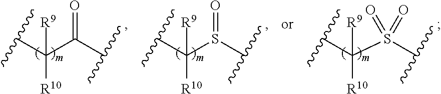

n and m can be independently an integer from 0 to 6, from 0 to 5, from 0 to 4, from 0 to 3, or from 0 to 2, such as 1 or 0; R⁶ can be a halogen (e.g., fluorine, chlorine, bromine, or iodine), a cyano, an azido, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, or a substituted or unsubstituted heterocyclyl; and R⁷—R¹⁰ can be independently a hydrogen, a substituted or unsubstituted alkyl, a substituted or unsubstituted alkenyl, a substituted or unsubstituted alkynyl, a halogen, a hydroxyl, an amino, an amido, an azido, an alkoxy, an azo, a polyether, a thiol, a cyano, a nitro, a nitrile, a carbonyl, a sulfinyl, a sulfonyl, or a sulfamoyl.

In some forms of Formula I, R² can be

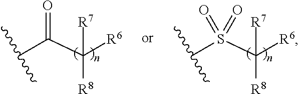

and wherein: n can be an integer from 0 to 3, from 0 to 2, 0 or 1; R⁶ can be a halogen (e.g., fluorine, chlorine, bromine, or iodine), a cyano, an azido, a substituted or unsubstituted alkenyl, or a substituted or unsubstituted alkynyl; and R⁷ and R⁸ can be independently a hydrogen, a substituted or unsubstituted alkyl, a substituted or unsubstituted alkenyl, a substituted or unsubstituted alkynyl, a halogen, a cyano, or an azido.

In some forms of Formula I, R² can be

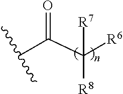

and wherein: n can be an integer from 0 to 3, from 0 to 2, 0 or 1; R⁶ can be a halogen (e.g., fluorine, chlorine, bromine, or iodine), a cyano, an azido, a substituted or unsubstituted alkenyl, or a substituted or unsubstituted alkynyl, such as a chlorine, an unsubstituted ethylenyl, an unsubstituted propylenyl, an unsubstituted ethynyl, or an unsubstituted propynyl; and R⁷ and R⁸ can be independently a hydrogen, a substituted or unsubstituted alkyl, a substituted or unsubstituted alkenyl, a substituted or unsubstituted alkynyl, a halogen, a cyano, or an aizdo, such as hydrogen.

In some forms of Formula I, R² can be

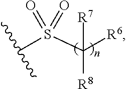

and wherein: n can be an integer from 0 to 3, from 0 to 2, 0 or 1; R⁶ can be a halogen (e.g., fluorine, chlorine, bromine, or iodine), a cyano, an azido, a substituted or unsubstituted alkenyl, or a substituted or unsubstituted alkynyl, such as a chlorine, an unsubstituted ethylenyl, an unsubstituted propylenyl, an unsubstituted ethynyl, or an unsubstituted propynyl; and R⁷ and R⁸ can be independently a hydrogen, a substituted or unsubstituted alkyl, a substituted or unsubstituted alkenyl, a substituted or unsubstituted alkynyl, a halogen, a cyano, or an aizdo, such as hydrogen.

In some forms, R⁶ can be a halogen (e.g., fluorine, chlorine, bromine, or iodine), an azido, an unsubstituted C₂-C₆ alkenyl, or an unsubstituted C₂-C₆ alkynyl, such as a chlorine, an azido, an unsubstituted ethylenyl, an unsubstituted propylenyl, an unsubstituted ethynyl, or an unsubstituted propynyl. In some forms of Formula I, R⁶ can be fluorine, bromine, iodine, an azido, an unsubstituted C₃-C₆ alkenyl, or an unsubstituted C₂-C₆ alkynyl.

In some forms of Formula I, R¹ and R¹′ can be independently a hydrogen,

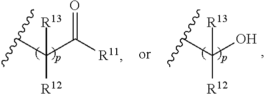

wherein at least one of R¹ and R¹′ is not hydrogen, and wherein: p can be an integer from 0 to 6, from 0 to 5, from 0 to 4, from 1 to 5, from 2 to 5, from 1 to 4, or from 2 to 4, such as 3; and R¹¹_R¹³ can be independently a hydrogen, a substituted or unsubstituted alkyl, a substituted or unsubstituted alkenyl, a substituted or unsubstituted alkynyl, a halogen, a hydroxyl, an amino, an amido, an alkoxy, an azo, an azido, a polyether, a thiol, a cyano, a nitro, or a nitrile.

In some forms of Formula I, R¹ and R¹′ can be independently

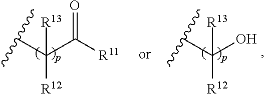

and wherein: p can be an integer from 0 to 6, from 0 to 5, from 0 to 4, from 1 to 5, from 2 to 5, from 1 to 4, or from 2 to 4, such as 3; and R¹¹—R¹³ can be independently a hydrogen, a substituted or unsubstituted alkyl, a substituted or unsubstituted alkenyl, a substituted or unsubstituted alkynyl, a halogen, a hydroxyl, an amino, an amido, an alkoxy, an azo, an azido, a polyether, a thiol, a cyano, a nitro, or a nitrile.

In some forms, R¹¹ can be a hydrogen, a hydroxyl, or a substituted or unsubstituted alkyl; and R¹² and R¹³ can be independently a hydrogen or a substituted or unsubstituted alkyl.

In some forms, R¹¹ can be an unsubstituted C₁-C₆ alkyl, an unsubstituted C₁-C₅ alkyl, an unsubstituted C₁-C₄ alkyl, an unsubstituted C₁-C₃ alkyl, an unsubstituted methyl or ethyl, such as methyl.

In some forms of Formula I, R¹ and R¹′ can be independently a hydrogen or

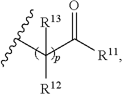

wherein at least one of R¹ and R¹′ is not hydrogen, wherein p can be an integer from 1 to 5, from 2 to 5, from 1 to 4, or from 2 to 4, such as 3; R¹¹ can be an unsubstituted C₁-C₆ alkyl, an unsubstituted C₁-C₅ alkyl, an unsubstituted C₁-C₄ alkyl, an unsubstituted C₁-C₃ alkyl, an unsubstituted methyl or ethyl, such as methyl; and R¹² and R¹³ can be independently a hydrogen or a substituted or unsubstituted alkyl, such as hydrogen.

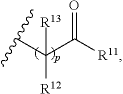

In some forms of Formula I, R¹ and R¹′ can be independently wherein p can be an integer from 1 to 5, from 2 to 5, from 1 to 4, or from 2 to 4, such as 3; R¹¹ can be an unsubstituted C₁-C₆ alkyl, an unsubstituted C₁-C₅ alkyl, an unsubstituted C₁-C₄ alkyl, an unsubstituted C₁-C₃ alkyl, an unsubstituted methyl or ethyl, such as methyl; and R¹² and R¹³ can be independently a hydrogen or a substituted or unsubstituted alkyl, such as hydrogen.

In some forms, R¹² and R¹³ can be hydrogen.

In some forms, R⁵ can be hydrogen.

In some forms of Formula I, R³, R⁴, R²′, R³′, and R⁴′ can be independently a hydrogen or a substituted or unsubstituted alkyl.

In some forms of Formula I, R³, R⁴, R²′, R³′, and R⁴′ can be hydrogen.

For any of the forms of Formula I described above, when any of R¹—R¹³ is a substituted functional group, the substituents can be a substituted or unsubstituted alkyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, an amino, an amido, an azido, or an oxo, or a combination thereof.

For any of the forms of Formula I described above, when any of R¹—R¹³ is a substituted functional group, the substituents can be an unsubstituted alkyl, an unsubstituted heterocyclyl, an unsubstituted aryl, an unsubstituted heteroaryl, an unsubstituted polyaryl, an unsubstituted polyheteroaryl, an unsubstituted aralkyl, a carbonyl, an alkoxy, a halogen, a hydroxyl, an amino, an amido, an azido, or an oxo, or a combination thereof.

For any of the forms of Formula I described above, when any of R¹—R¹³ is an alkyl, the alkyl can be a linear alkyl, a branched alkyl, or a cyclic alkyl (either monocyclic or polycyclic). Exemplary alkyl includes a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, C₁-C₂ alkyl group, a branched C₃-C₉, C₃-C₉, C₃-C₈, C₃-C₇, C₃-C₆, C₃-C₅, C₃-C₄ alkyl group, or a cyclic C₃-C₉, C₃-C₉, C₃-C₈, C₃-C₇, C₃-C₆, C₃-C₅, C₃-C₄ alkyl group.

For any of the forms of Formula I described above, when any of R¹—R¹³ is an alkenyl, the alkenyl can be a linear alkenyl, a branched alkenyl, or a cyclic alkenyl (either monocyclic or polycyclic). Exemplary alkenyl includes a linear C₂-C₃₀ alkenyl, a branched C₄-C₃₀ alkenyl, a cyclic C₃-C₃₀ alkenyl, a linear C₂-C₂₀ alkenyl, a branched C₄-C₂₀ alkenyl, a cyclic C₃-C₂₀ alkenyl, a linear C₂-C₁₀ alkenyl, a branched C₄-C₁₀ alkenyl, a cyclic C₃-C₁₀ alkenyl, a linear C₂-C₆ alkenyl, a branched C₄-C₆ alkenyl, a cyclic C₃-C₆ alkenyl, a linear C₂-C₄ alkenyl, cyclic C₃-C₄ alkenyl, such as a linear C₂-C₁₀, C₂-C₉, C₂-C₈, C₂-C₇, C₂-C₆, C₂-C₅, C₂-C₄, C₂-C₃ alkenyl group, a branched C₃-C₉, C₃-C₉, C₃-C₈, C₃-C₇, C₃-C₆, C₃-C₅, C₃-C₄ alkenyl group, or a cyclic C₃-C₉, C₃-C₉, C₃-C₈, C₃-C₇, C₃-C₆, C₃-C₅, C₃-C₄ alkenyl group.

For any of the forms of Formula I described above, when any of R¹—R¹³ is an alkynyl, the alkynyl can be a linear alkynyl, a branched alkynyl, or a cyclic alkynyl (either monocyclic or polycyclic). Exemplary alkynyl includes a linear C₂-C₃₀ alkynyl, a branched C₄-C₃₀ alkynyl, a cyclic C₃-C₃₀ alkynyl, a linear C₂-C₂₀ alkynyl, a branched C₄-C₂₀ alkynyl, a cyclic C₃-C₂₀ alkynyl, a linear C₂-C₁₀ alkynyl, a branched C₄-C₁₀ alkynyl, a cyclic C₃-C₁₀ alkynyl, a linear C₂-C₆ alkynyl, a branched C₄-C₆ alkynyl, a cyclic C₃-C₆ alkynyl, a linear C₂-C₄ alkynyl, cyclic C₃-C₄ alkynyl, such as a linear C₂-C₁₀, C₂-C₉, C₂-C₈, C₂-C₇, C₂-C₆, C₂-C₅, C₂-C₄, C₂-C₃ alkynyl group, a branched C₃-C₉, C₃-C₉, C₃-C₈, C₃-C₇, C₃-C₆, C₃-C₅, C₃-C₄ alkynyl group, or a cyclic C₃-C₉, C₃-C₉, C₃-C₈, C₃-C₇, C₃-C₆, C₃-C₅, C₃-C₄ alkynyl group.

It is understood that any of the exemplary alkyl, alkenyl, and alkynyl groups described above can be heteroalkyl, heteroalkenyl, and heteroalkynyl, respectively. For example, the alkyl can be a linear C₂-C₃₀ heteroalkyl, a branched C₄-C₃₀ heteroalkyl, a cyclic C₃-C₃₀ heteroalkyl (i.e. a heterocycloalkyl), a linear C₁-C₂₀ heteroalkyl, a branched C₄-C₂₀ heteroalkyl, a cyclic C₃-C₂₀ heteroalkyl, a linear C₁-C₁₀ heteroalkyl, a branched C₄-C₁₀ heteroalkyl, a cyclic C₃-C₁₀ heteroalkyl, a linear C₁-C₆ heteroalkyl, a branched C₄-C₆ heteroalkyl, a cyclic C₃-C₆ heteroalkyl, a linear C₁-C₄ heteroalkyl, cyclic C₃-C₄ heteroalkyl, such as a linear C₁-C₁₀, C₁-C₉, C₁-C₈, C₁-C₇, C₁-C₆, C₁-C₅, C₁-C₄, C₁-C₃, C₁-C₂ heteroalkyl group, a branched C₃-C₉, C₃-C₉, C₃-C₈, C₃-C₇, C₃-C₆, C₃-C₅, C₃-C₄ heteroalkyl group, or a cyclic C₃-C₉, C₃-C₉, C₃-C₈, C₃-C₇, C₃-C₆, C₃-C₅, C₃-C₄ heteroalkyl group. For example, the alkenyl can be a linear C₂-C₃₀ heteroalkenyl, a branched C₄-C₃₀ heteroalkenyl, a cyclic C₃-C₃₀ heteroalkenyl (i.e., a heterocycloalkenyl), a linear C₂-C₂₀ heteroalkenyl, a branched C₄-C₂₀ heteroalkenyl, a cyclic C₃-C₂₀ heteroalkenyl, a linear C₂-C₁₀ heteroalkenyl, a branched C₄-C₁₀ heteroalkenyl, a cyclic C₃-C₁₀ heteroalkenyl, a linear C₂-C₆ heteroalkenyl, a branched C₄-C₆ heteroalkenyl, a cyclic C₃-C₆ heteroalkenyl, a linear C₂-C₄ heteroalkenyl, cyclic C₃-C₄ heteroalkenyl, such as a linear C₂-C₁₀, C₂-C₉, C₂-C₈, C₂-C₇, C₂-C₆, C₂-C₅, C₂-C₄, C₂-C₃ heteroalkenyl group, a branched C₃-C₉, C₃-C₉, C₃-C₈, C₃-C₇, C₃-C₆, C₃-C₅, C₃-C₄ heteroalkenyl group, or a cyclic C₃-C₉, C₃-C₉, C₃-C₈, C₃-C₇, C₃-C₆, C₃-C₅, C₃-C₄ heteroalkenyl group. For example, the alkynyl can be a linear C₂-C₃₀ heteroalkynyl, a branched C₄-C₃₀ heteroalkynyl, a cyclic C₃-C₃₀ heteroalkynyl (i.e., a heterocycloalkynyl), a linear C₂-C₂₀ heteroalkynyl, a branched C₄-C₂₀ heteroalkynyl, a cyclic C₃-C₂₀ heteroalkynyl, a linear C₂-C₁₀ heteroalkynyl, a branched C₄-C₁₀ heteroalkynyl, a cyclic C₃-C₁₀ heteroalkynyl, a linear C₂-C₆ heteroalkynyl, a branched C₄-C₆ heteroalkynyl, a cyclic C₃-C₆ heteroalkynyl, a linear C₂-C₄ heteroalkynyl, cyclic C₃-C₄ heteroalkynyl, such as a linear C₂-C₁₀, C₂-C₉, C₂-C₈, C₂-C₇, C₂-C₆, C₂-C₅, C₂-C₄, C₂-C₃ heteroalkynyl group, a branched C₃-C₉, C₃-C₉, C₃-C₈, C₃-C₇, C₃-C₆, C₃-C₅, C₃-C₄ heteroalkynyl group, or a cyclic C₃-C₉, C₃-C₉, C₃-C₅, C₃-C₇, C₃-C₆, C₃-C₅, C₃-C₄ heteroalkynyl group.

For any of the forms of Formula I described above, when any of R¹—R¹³ is an aryl, the aryl group can be a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁₁ aryl, or a C₆-C₉ aryl. It is understood that the aryl can be a heteroaryl, such as a C₅-C₃₀ heteroaryl, a C₅-C₂₀ heteroaryl, a C₅-C₁₂ heteroaryl, a C₅-C₁₁ heteroaryl, a C₅-C₉ heteroaryl, a C₆-C₃₀ heteroaryl, a C₆-C₂₀ heteroaryl, a C₆-C₁₂ heteroaryl, a C₆-C₁₁ heteroaryl, or a C₆-C₉ heteroaryl. The polyaryl group can be a C₁₀-C₃₀ polyaryl, a C₁₀-C₂₀ polyaryl, a C₁₀-C₁₂ polyaryl, a C₁₀-C₁₁ polyaryl, or a C₁₂-C₂₀ polyaryl. It is understood that the aryl can be a polyheteroaryl, such as a C₁₀-C₃₀ polyheteroaryl, a C₁₀-C₂₀ polyheteroaryl, a C₁₀-C₁₂ polyheteroaryl, a C₁₀-C₁₁ polyheteroaryl, or a C₁₂-C₂₀ polyheteroaryl.

The compounds may contain one or more chiral centers or may otherwise be capable of existing as multiple stereoisomers. These may be pure (single) stereoisomers or mixtures of stereoisomers, such as enantiomers, diastereomers, and enantiomerically or diastereomerically enriched mixtures. The compounds may be capable of existing as geometric isomers. Accordingly, it is to be understood that the present invention includes pure geometric isomers or mixtures of geometric isomers.

### 2. Exemplary Compounds

Exemplary compounds are presented below.

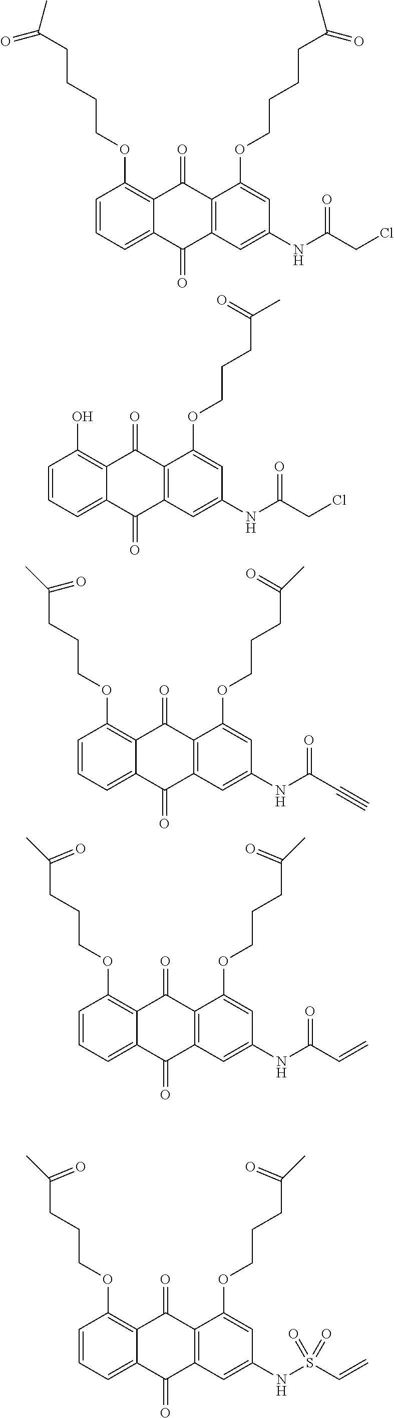

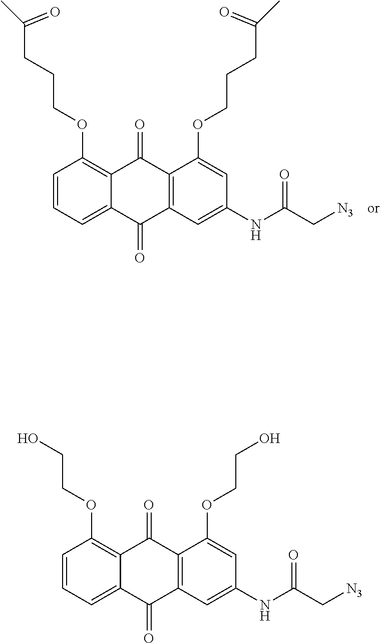 [SMILES: CC(=O)CCCOc1cccc2c1C(=O)c1c(OCCCC(C)=O)cc(NC(=O)CN=[N+]=[N-])cc1C2=O ; [N-]=[N+]=NCC(=O)Nc1cc(OCCO)c2c(c1)C(=O)c1cccc(OCCO)c1C2=O]

In some forms, the compound is not

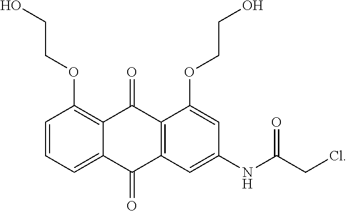 [SMILES: O=C(CCl)Nc1cc(OCCO)c2c(c1)C(=O)c1cccc(OCCO)c1C2=O]

In some forms, the compound is not

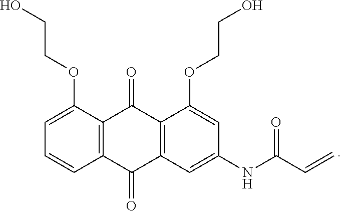 [SMILES: C=CC(=O)Nc1cc(OCCO)c2c(c1)C(=O)c1cccc(OCCO)c1C2=O]

In some forms, the compound is not

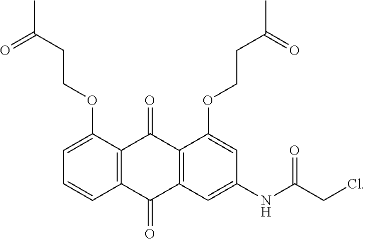 [SMILES: CC(=O)CCOc1cccc2c1C(=O)c1c(OCCC(C)=O)cc(NC(=O)CCl)cc1C2=O]

In some forms, the compound is not

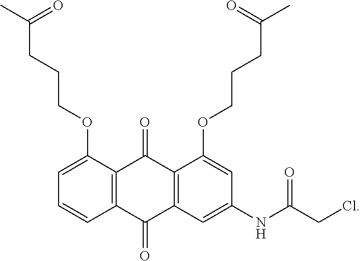 [SMILES: CC(=O)CCCOc1cccc2c1C(=O)c1c(OCCCC(C)=O)cc(NC(=O)CCl)cc1C2=O]

In some forms, the compound is not

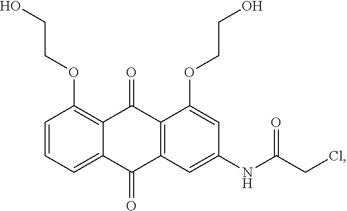 [SMILES: O=C(CCl)Nc1cc(OCCO)c2c(c1)C(=O)c1cccc(OCCO)c1C2=O]

not

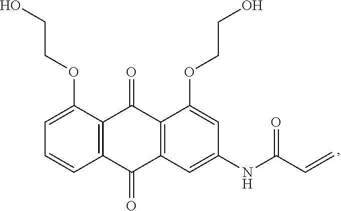 [SMILES: C=CC(=O)Nc1cc(OCCO)c2c(c1)C(=O)c1cccc(OCCO)c1C2=O]

not

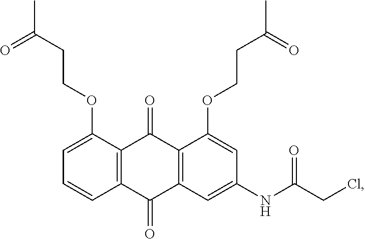 [SMILES: CC(=O)CCOc1cccc2c1C(=O)c1c(OCCC(C)=O)cc(NC(=O)CCl)cc1C2=O]

and not

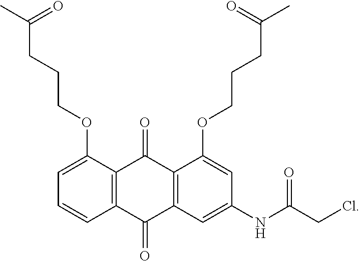 [SMILES: CC(=O)CCCOc1cccc2c1C(=O)c1c(OCCCC(C)=O)cc(NC(=O)CCl)cc1C2=O]

In some forms, when R¹ and/or R¹′ contains a terminal hydroxyl group, the compound may have similar cytoxoticity compared to compounds that do not have hydroxyl terminal groups at the R¹ and/or R¹′ positions, and may have increased solubility and/or different pharmacokinetics profile compared to compounds that do not have hydroxyl terminal groups at the R¹ and/or R¹′ positions. For example, when the compound

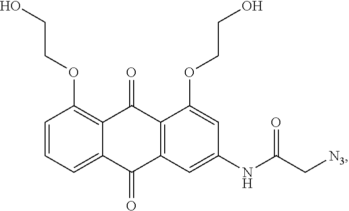 [SMILES: [N-]=[N+]=NCC(=O)Nc1cc(OCCO)c2c(c1)C(=O)c1cccc(OCCO)c1C2=O]

is it may have a similar cytotoxicity compared to one or more compounds exemplified above that do not have a terminal hycrosyl at the R¹ and/or R¹′ positions, and may have an increased solubility and/or different pharmacokinetics profile compared to one or more compounds exemplified above that do not have hydroxyl terminal groups at the R¹ and/or R¹′ positions.

### 3. Pharmaceutically Acceptable Salts

The compounds may be neutral or may be one or more pharmaceutically acceptable salts, crystalline forms, non crystalline forms, hydrates, or solvates, or a combination thereof. References to the compounds may refer to the neutral molecule, and/or those additional forms thereof collectively and individually from the context. Pharmaceutically acceptable salts of the compounds include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

### B. Pharmaceutical Formulations

Pharmaceutical formulations that contain one or more the compounds disclosed herein, in a form suitable for administration to a mammal, are disclosed. Typically, the compound(s) in the pharmaceutical formulation is present in an amount effective to treat or ameliorate one or more symptoms associated with a cancer in a subject. In some forms, the compound(s) in the pharmaceutical formulation is present in an amount effective to induce apoptosis of cancer cells in the subject. In some forms, the cancer cells are cancer cells harboring WT-p53, and the effective amount of the one or more compounds is effective to downregulate MDM2 and upregulate p53 in the cancer cells compared to the cancer cells before treatment as shown by Western blot. The cancer cells harboring WT-p-53 can be leukemia cells harboring WT-p53 (e.g. EU-1 leukemia cells), MCF7 cells, RS4;11 cells, or HeLa cells, or a combination thereof.

The pharmaceutical formulation containing the compound(s) may also include one or more pharmaceutically acceptable carriers and/or one or more pharmaceutically acceptable excipients. For example, the pharmaceutical formulation may be in the form of a liquid, such as a solution or a suspension, and contain one or more the disclosed compounds in an aqueous medium and, optionally, one or more suitable excipients for the liquid formulation. Optionally, the pharmaceutical formulation is in a solid form, and contains one or more the disclosed compounds and one or more suitable excipients for a solid formulation.

The pharmaceutical formulation may include a second active agent, optionally more than one second active agent. The second active agent can be an anticancer agent that is different from the compounds disclosed herein.

### 1. Carriers and Excipients

The pharmaceutical formulation can contain one or more pharmaceutically acceptable carriers and/or one or more pharmaceutically acceptable excipients. Suitable pharmaceutically acceptable carriers and excipients are generally recognized as safe (GRAS), and may be administered to an individual without causing undesirable biological side effects or unwanted interactions.

Representative carriers and excipients that can be used in the pharmaceutical formulations include solvents (including buffers), diluents, pH modifying agents, preservatives, antioxidants, suspending agents, wetting agents, viscosity modifiers, tonicity agents, and stabilizing agents, and a combination thereof.

In some forms, the compounds can be dissolved or suspended in a suitable carrier to form a liquid pharmaceutical formulation, such as sterile saline, phosphate buffered saline (PBS), balanced salt solution (BSS), viscous gel, or other pharmaceutically acceptable carriers for administration. The pharmaceutical formulation may also be a sterile solution, suspension, or emulsion in a nontoxic, parenterally acceptable diluent or solvent.

Excipients can be added to a liquid or solid pharmaceutical formulation to assist in sterility, stability (e.g. shelf-life), integration, and to adjust and/or maintain pH or isotonicity of the compounds in the pharmaceutical formulation, such as diluents, pH modifying agents, preservatives, antioxidants, suspending agents, wetting agents, viscosity modifiers, tonicity agents, and stabilizing agents, and a combination thereof.

### 2. Form

The pharmaceutical formulation containing one or more the disclosed compounds can be in a liquid form or a solid form, as a liquid formulation or a solid formulation for oral administration or parenteral administration (e.g. intramuscular administration, intravenous administration, intraperitoneal administration, and subcutaneous administration) to a subject.

### a. Oral Formulations

In some forms, the pharmaceutical formulation containing one or more the disclosed compounds can be in a form suitable for oral administration to a subject, such as a mammal (i.e. an oral formulation). Oral administration may involve swallowing, so that the compound(s) enter the gastrointestinal tract, or buccal or sublingual administration may be employed by which the compound(s) enter(s) the blood stream directly from the mouth.

Formulations suitable for oral administration include solid formulations such as tablets, capsules containing particulates, liquids, powders, lozenges (including liquid-filled lozenges), chews, multi- and nano-particulates, gels, solid solutions, liposomes, films, ovules, sprays, and liquid formulations.

Liquid formulations for oral administration include suspensions, solutions, syrups, and elixirs. Such oral formulations may be employed as fillers in soft or hard capsules and can contain one or more suitable carriers and/or excipients, for example, water, ethanol, polyethylene glycol, propylene glycol, chitosan polymers and chitosan derivatives (e.g. N-trimethylene chloride chitosan, chitosan esters, chitosan modified with hydrophilic groups, such as amino groups, carboxyl groups, sulfate groups, etc.), methylcellulose, a suitable oil, one or more emulsifying agents, and/or suspending agents. Liquid formulations for oral administration may also be prepared by the reconstitution of a solid, for example, from a sachet.

Optionally, the compound(s) is/are included in a fast-dissolving and/or fast-disintegrating dosage form.

For tablet or capsule dosage forms, in addition to the compound(s) described herein, tablets generally contain disintegrants, binders, diluents, surface active agents, lubricants, glidants, antioxidants, colourants, flavouring agents, preservatives, or taste masking agents, or a combination thereof.

Examples of suitable disintegrants for forming a table or capsule dosage form containing the compound(s) include, but are not limited to, sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant can have a concentration in a range from about 1 wt % to about 25 wt %, from about 5 wt % to about 20 wt % of the tablet or capsule dosage form containing the compound(s).

Binders are generally used to impart cohesive qualities to a tablet formulation. Suitable binders for forming a tablet or capsule formulation containing the compound(s) include, but are not limited to, microcrystalline cellulose, gelatin, sugars, polyethylene glycol, natural and synthetic gums, polyvinylpyrrolidone, pregelatinised starch, chitosan polymers and chitosan derivatives (e.g. N-trimethylene chloride chitosan, chitosan esters, chitosan modified with hydrophilic groups, such as amino groups, carboxyl groups, sulfate groups, etc.), hydroxypropyl cellulose, and hydroxypropyl methylcellulose.

Suitable diluents for forming a table or capsule formulation containing the compound(s) include, but are not limited to, lactose (as, for example, the monohydrate, spray-dried monohydrate or anhydrous form), chitosan polymers and chitosan derivatives (e.g. N-trimethylene chloride chitosan, chitosan esters, chitosan modified with hydrophilic groups, such as amino groups, carboxyl groups, sulfate groups, etc.), N-sulfonated derivatives of chitosan, quaternarized derivatives of chitosan, carbosyalkylated chitosan, microcrystalline chitosan, mannitol, xylitol, dextrose, sucrose, sorbitol, microcrystalline cellulose, starch and dibasic calcium phosphate dihydrate.

Tablet or capsule formulations containing the compound(s) may also contain surface active agents, such as sodium lauryl sulfate and polysorbate 80, and glidants such as silicon dioxide and talc. When present, surface active agents can have a concentration in a range from about 0.2 w1% to 5 w1% of the tablet or capsule formulation.

Tablet or capsule formulations containing the compound(s) also can contain lubricants, such as magnesium stearate, calcium stearate, zinc stearate, sodium stearyl fumarate, and mixtures of magnesium stearate with sodium lauryl sulphate. Lubricants can have a concentration in a range from about 0.25 wt % to 10 wt %, from about 0.5 wt % to about 3 wt % of the tablet or capsule formulation.

Other possible excipients included in a tablet or capsule formulation containing the compound(s) include glidants (e.g., Talc or colloidal anhydrous silica at about 0.1 wt % to about 3 wt % of the table or capsule formulation), antioxidants, colourants, flavouring agents, preservatives and taste-masking agents. When present, glidants can have a concentration in a range from about 0.2 wt % to 1 wt % of the tablet or capsule formulation.

An exemplary tablet formulation contains up to about 80 wt % of the compound(s) described herein, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet or capsule blends, including the compound(s) and one or more suitable excipients, may be compressed directly or by roller to form tablets. Tablet or capsule blends or portions of the blends may alternatively be wet-, dry-, or melt-granulated, melt congealed, or extruded before tableting. The final table or capsule formulation may contain one or more layers and may be coated or uncoated; it may even be encapsulated in a particle, such as a polymeric particle or a liposomal particle.

Solid formulations containing the compound(s) for oral administration may be formulated to be immediate and/or modified release. Modified release formulations include delayed, sustained, pulsed, controlled, targeted and programmed release formulations.

### b. Parenteral Formulations

In some forms, the pharmaceutical formulation containing one or more the disclosed compounds can be in a form suitable for administration directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intramuscular, and subcutaneous delivery. Suitable means for parenteral administration include needle (including microneedle) injectors, needle-free injectors, and infusion techniques.

For example, the pharmaceutical formulation containing one or more the compounds are in a form suitable for intramuscular administration, intravenous administration, intraperitoneal administration, or subcutaneous administration, or a combination thereof.

Parenteral formulations containing the compound(s) described herein are typically aqueous solutions which can contain excipients such as salts, carbohydrates and buffering agents (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4), but, for some applications, they may be more suitably formulated as a sterile aqueous solution or as a dried form to be used in conjunction with a suitable vehicle such as sterile, pyrogen-free water.

The liquid formulations containing the compound(s) for parenteral administration may be a solution, a suspension, or an emulsion.

The liquid pharmaceutically acceptable carrier forming the parenteral formulation containing the compound(s) can include one or more physiologically compatible buffers, such as a phosphate buffer. One skilled in the art can readily determine a suitable saline content and pH for an aqueous carrier for administration (e.g., from about pH 6.5 to about pH 8.0, from about pH 6.5 to about pH 7.4, from about pH 6.5 to about pH 7.0, from about pH 7.0 to pH 8.0, or from about pH 7.0 to about pH 7.4).

Liquid formulations containing the compound(s) for parenteral administration may include one or more suspending agents, such as cellulose derivatives, sodium alginate, polyvinylpyrrolidone, gum tragacanth, or lecithin. The liquid formulations may also include one or more preservatives, such as ethyl or n-propyl p-hydroxybenzoate.

In some forms, the liquid formulation containing the compound(s) contains one or more solvents that are low toxicity organic (i.e., nonaqueous) class 3 residual solvents, such as ethanol, acetone, ethyl acetate, tetrahydofuran, ethyl ether, and propanol, and a combination thereof. Any such solvents included in the liquid formulation should not detrimentally react with the compound(s) and any additional active agents when present in the liquid formulation. Solvents such as freon, alcohol, glycol, polyglycol, or fatty acid, can also be included in the liquid formulation containing the compound(s) as desired to increase the volatility of the solution or suspension.

Liquid formulations containing the compound(s) for parenteral administration may also contain minor amounts of polymers, surfactants, or other pharmaceutically acceptable excipients known to those in the art. In this context, “minor amounts” means an amount that is sufficiently small to avoid adversely affecting uptake of the compounds by the targeted cells, such as pituitary gonadotrophs.

The preparation of parenteral formulations containing the compound(s) is typically under sterile conditions, for example, by lyophilisation, which can be accomplished using standard pharmaceutical techniques known to those skilled in the art.

Formulations for parenteral administration containing the compound(s) may be formulated to provide immediate and/or modified release of the active agent. Modified release formulations include delayed, sustained, pulsed, controlled, targeted and programmed release formulations.

### c. Pulmonary and Mucosal Formulations

In some forms, the pharmaceutical formulation containing one or more the disclosed compounds can be in a form suitable for pulmonary or mucosal administration. The administration can include delivery of the composition to the lungs, nasal, oral (sublingual, buccal), vaginal, or rectal mucosa.

For example, the compounds can be administered intranasally or by oral inhalation, such as in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (such as an atomiser using electrohydrodynamics to produce a fine mist), or nebuliser, with or without the use of a suitable propellant, such as water, ethanol-water mixture, 1,1,1,2-tetrafluoroethane or 1,1,1,2,3,3,3-heptafluoropropane. For intranasal or oral inhalation use, the powder may comprise a bioadhesive agent, for example, chitosan or cyclodextrin. The term aerosol as used herein refers to any preparation of a fine mist of particles, which can be in solution or a suspension, whether or not it is produced using a propellant. Aerosols can be produced using standard techniques, such as ultrasonication or high-pressure treatment.

The pressurized container, pump, spray, atomizer, or nebuliser contains a solution or suspension of one or more of the compounds including, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, a drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compounds described herein, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose, and trehalose.

A suitable solution formulation containing the compound(s) for use in an atomizer using electrohydrodynamics to produce a fine mist may contain from 1 μg to 20 mg of one or more of the compounds per actuation and the actuation volume may vary from 1 μl to 100 μl. A typical formulation may contain one or more of the compounds described herein, propylene glycol, sterile water, ethanol and sodium chloride. Alternative solvents that may be used instead of propylene glycol include glycerol and polyethylene glycol. Suitable flavors, such as menthol and levomenthol, or sweeteners, such as saccharin or saccharin sodium, may be added to those formulations intended for inhaled/intranasal administration.

Formulations for inhaled/intranasal administration may be formulated to be immediate and/or modified release using, for example, PGLA. Modified release formulations include delayed, sustained, pulsed, controlled, targeted, and programmed release formulations.

In the case of dry powder inhalers and aerosols, the dosage unit is determined by means of a valve which delivers a metered amount. Units in accordance with the compounds are typically arranged to administer a metered dose or “puff.” The overall daily dose will be administered in a single dose or, more usually, as divided doses throughout the day.

In some forms, the compounds can be formulated for pulmonary delivery, such as intranasal administration or oral inhalation. Carriers for pulmonary formulations can be divided into those for dry powder formulations and for administration as solutions. For administration via the upper respiratory tract, the formulation can be formulated into an aqueous solution, e.g., water or isotonic saline, buffered or un-buffered, or as an aqueous suspension, for intranasal administration as drops or as a spray. Such aqueous solutions or suspensions may be isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. One skilled in the art can readily determine a suitable saline content and pH for an innocuous aqueous solution for nasal and/or upper respiratory administration.

In some forms, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In some forms, solvents that are low toxicity organic (i.e., nonaqueous) class 3 residual solvents, such as ethanol, acetone, ethyl acetate, tetrahydrofuran, ethyl ether, and propanol may be used for the formulations containing the compound(s). The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In some forms, the pharmaceutical formulations containing the compound(s) may contain minor amounts of polymers, surfactants, or other excipients well known to those of the art. In this context, “minor amounts” means no excipients are present that might affect or mediate uptake of the compounds by cells and that the excipients that are present in amount that do not adversely affect uptake of compounds by cells.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Non-aqueous suspensions of lipids can also be prepared in absolute ethanol using a reusable PARI LC Jet+nebulizer (PARI Respiratory Equipment, Monterey, CA).

d. Topical Formulations

The compounds can be administered directly to the external surface of the skin or the mucous membranes (including the surface membranes of the nose, lungs and mouth), such that the compounds can cross the external surface of the skin or mucous membrane and enters the underlying tissues.

Formulations for topical administration generally contain a dermatologically acceptable carrier that is suitable for application to the skin, has good aesthetic properties, is compatible with the active agents and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse or a transdermal patch. Other suitable topical carriers include anhydrous liquid solvents such as oils, alcohols, and silicones (e.g., mineral oil, ethanol isopropanol, dimethicone, cyclomethicone, and the like); aqueous-based single phase liquid solvents (e.g., hydro-alcoholic solvent systems, such as a mixture of ethanol and/or isopropanol and water); and thickened versions of these anhydrous and aqueous-based single phase solvents (e.g. where the viscosity of the solvent has been increased to form a solid or semi-solid by the addition of appropriate gums, resins, waxes, polymers, salts, and the like). Examples of topical carrier systems useful in the present formulations are described in the following four references all of which are incorporated herein by reference in their entirety: “Sun Products Formulary” Cosmetics & Toiletries, vol. 105, pp. 122-139 (December 1990); “Sun Products Formulary,” Cosmetics &

Toiletries, vol. 102, pp. 117-136 (March 1987); U.S. Pat. No. 5,605,894 to Blank et al., and U.S. Pat. No. 5,681,852 to Bissett.

Formulations containing the compound(s) for topical administration may be formulated to be immediate and/or modified release. Modified release formulations include delayed, sustained, pulsed, controlled, targeted and programmed release formulations. Thus, the compounds may be formulated as a solid, semi-solid, or thixotropic liquid for administration as an implanted depot providing modified release of the compounds. Examples of such formulations include drug-coated stents and poly (dl-lactic-coglycolic) acid (PGLA) microspheres.

### 3. Additional Active Agent(s)

In some forms, the pharmaceutical formulation can include one or more additional active agents, such as one or more additional anticancer agents. Anticancer agents that can be included in the pharmaceutical compositions or formulations are known, for example, see the National Cancer Institute database, “A to Z List of Cancer Drugs,” website cancer.gov/about-cancer/treatment/drugs.

Exemplary anticancer drugs that can be included in the pharmaceutical formulation containing the compound(s) include, but are not limited to, doxorubicin, olaparib, abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

### 4. Dosages/wt % Concentration

In some forms, the pharmaceutical formulation contains an effective amount of the comopund(s) for treating or ameliorating one or more symptoms associated with a cancer in a subject.

In some forms, the compound(s) in the pharmaceutical formulation is present in an amount effective to induce apoptosis of cancer cells in the subject. In some forms, the cancer cells are cancer cells harboring WT-p53, and the effective amount of the one more compounds is effective to downregulate MDM2 and upregulate p53 in the cancer cells compared to the cancer cells before treatment as shown by Western blot. The cancer cells harboring WT-p-53 can be leukemia cells harboring WT-p53 (e.g. EU-1 leukemia cells), MCF7 cells, RS4;11 cells, or HeLa cells, or a combination thereof.

The total concentration of the compound(s) in the pharmaceutical formulation can be at least 0.001 wt %, at least 0.005 wt %, at least 0.01 wt %, at least 0.05 wt %, at least 0.1 wt %, in a range from 0.001 wt % to 50 wt %, from 0.005 wt % to 50 wt %, from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.001 wt % to 10 wt %, from 0.005 wt % to 10 wt %, from 0.001 wt % to 1 wt %, from 0.005 wt % to 5 wt %, from 0.1 w1% to 20 wt %, from 0.01 wt % to 10 wt %, from 0.05 wt % to 10 wt %, or from 0.1 wt % to 10 wt %. The term “total concentration of the compound(s) in the pharmaceutical formulation” refers to the sum of the weight of all compound(s) relative to the weight of the formulation.

In some forms, the total concentration of the compound(s) in the pharmaceutical formulation that is effective to treat or ameliorate one or more symptoms associated with a cancer, such as leukemia, in a subject, can be at least 0.001 wt %, at least 0.005 wt %, at least 0.01 wt %, at least 0.05 wt %, at least 0.1 wt %, in a range from 0.001 wt % to 50 wt %, from 0.005 wt % to 50 wt %, from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 wt %, from 0.05 wt % to 20 wt %, from 0.001 wt % to 10 wt %, from 0.005 wt % to 10 wt %, from 0.001 wt % to 1 wt %, from 0.005 wt % to 5 wt %, from 0.1 wt % to 20 wt %, from 0.01 wt % to 10 wt %, from 0.05 wt % to 10 wt %, or from 0.1 wt % to 10 wt %.

In some forms, the total concentration of the compound(s) in the pharmaceutical formulation that is effective to induce apoptosis of cancer cells, such as cancer cells harboring WT-p53 in a subject can be at least 0.001 wt %, at least 0.005 wt %, at least 0.01 wt %, at least 0.05 wt %, at least 0.1 wt %, in a range from 0.001 wt % to 50 wt %, from 0.005 wt % to 50 wt %, from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 w1%, from 0.01 wt % to 30 wt %, from 0.05 wt % to 30 w1%, from 0.1 wt % to 30 w1%, from 0.01 wt % to 20 wt %, from 0.05 wt % to 20 wt %, from 0.001 wt % to 10 wt %, from 0.005 wt % to 10 wt %, from 0.001 wt % to 1 wt %, from 0.005 wt % to 5 wt %, from 0.1 wt % to 20 wt %, from 0.01 wt % to 10 wt %, from 0.05 wt % to 10 wt %, or from 0.1 wt % to 10 wt %.

In some forms, the total concentration of the compound(s) in the pharmaceutical formulation that is effective to downregulate MDM2 and/or upregulate p-53 in cancer cells harboring WT-p53 (such as leukemia cells harboring WT-p53 (e.g. EU-1 leukemia cells), MCF7 cells, RS4;11 cells, or HeLa cells, or a combination thereof) in a subject as shown by Wester blot can be at least 0.001 wt %, at least 0.005 wt %, at least 0.01 wt %, at least 0.05 wt %, at least 0.1 wt %, in a range from 0.001 wt % to 50 wt %, from 0.005 wt % to 50 wt %, from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 w1% to 30 wt %, from 0.05 wt % to 30 wt %, from 0.1 wt % to 30 wt %, from 0.01 wt % to 20 wt %, from 0.05 wt % to 20 wt %, from 0.001 wt % to 10 wt %, from 0.005 wt % to 10 wt %, from 0.001 wt % to 1 wt %, from 0.005 wt % to 5 wt %, from 0.1 wt % to 20 wt %, from 0.01 wt % to 10 wt %, from 0.05 wt % to 10 wt %, or from 0.1 wt % to 10 wt %.

In some forms, the pharmaceutical formulation containing the compound(s) can be provided in a unit dosage form. The dosage of the compounds in the pharmaceutical formulation in the unit dosage form can be in a range from about 0.002 mg to about 1 mg, in a range from about 0.006 mg to about 0.6 mg, in a range from about 0.01 mg to about 0.4 mg, in a range from about 0.02 mg to about 0.3 mg, or in a range from about 0.01 mg to about 0.2 mg.

### III. Methods of Making

The compounds can be synthesized using methods known in the art of organic synthesis, such as methods that use rhein or its methyl ester as the starting material in a suitable solvent medium to introduce functional groups at the R¹/R¹′ and R² positions, such as a hydroxyalkyl and/or an alkoxy group at the R¹ and/or R¹′ positions and/or an electrophilic group at the R² position.

For example, as shown in Example 1 below, the synthesis started with either rhein or its methyl ester. Alkylation with the corresponding alkenyl halide at the phenol hydroxyl groups of the anthraquinone core allowed for the introduction of the alkenyl group as a latent ketone moiety. Then the ester group of intermediates 1 was hydrolyzed before introducing an azido group on the free carboxylic acid 2, setting up a Curtis rearrangement for the subsequent formation of the arylamino group on 4. The introduction of various acyl or sulfonyl groups led to the installation of different R² in the target compounds. The conversion of the alkene moiety to a ketone group was conducted as the last step through Wacker oxidation for the synthesis of compounds with the chloroacetyl moiety (BW-AQ-260,-295,-345). For analogs with functional groups that are sensitive to Wacker oxidation, including BW-AQ-350,-353, and -354, the oxidative conversion of the alkenyl moiety to a ketone group was conducted as the penultimate step. Mono-substituted anthraquinone analogs, such as BW-AQ-336, can be synthesized by carefully controlling the alkylation conditions.

More specific methods for synthesizing exemplary compounds, are described in the Example below.

### IV. Methods For Using the Compounds

The disclosed compounds have anticancer properties and thereby can be used in methods for treating or ameliorating one or more symptoms associated with a cancer in a subject in need thereof, such as leukemia. In some forms, the compounds can be used in methods for treating cancer cells in a subject in need thereof.

A. Treating Ameliorating Symptom(s) Associated with Cancer

Methods of using the compounds for treating or ameliorating one or more symptoms associated with a cancer in a subject in need thereof are disclosed.

Generally, the method includes (i) administering to the subject a pharmaceutical formulation containing one or more of the compounds described above. The adminsitration step can occur one or more times.

The subject can be a mammal, such as a human, a dog, a cat, a rat, a monkey, rabbits, guinea pigs, etc., that is in need of cancer treatment. In some forms, the subject can be exhibiting symptoms of or diagnosed with cancer.

The pharmaceutical formulation can be administered by oral administration, parenteral administration, inhalation, mucosal administration, topical or a combination thereof. The compound(s) can be administered by a medical professional or the subject being treated (e.g. self-administration).

### 1. Cancers

The methods disclosed herein are particularly suitable for treating or ameliorating one more symptoms associated with leukemia, such as Acute lymphocytic leukemia (ALL), Acute myelogenous leukemia (AML), Chronic lymphocytic leukemia (CLL), or Chronic myelogenous leukemia (CML). However, other cancers may be treated using the disclosed methods to treat or ameliorate one or more more symptoms of those cancers.

In some forms of the method, the cancer being treated can be tumors, such as tumors of the hematopoietic and lymphoid tissues or hematopoietic and lymphoid malignancies, tumors that affect the blood, bone marrow, lymph, and lymphatic system, and tumors located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax, and genito-urinary apparatus.

In some forms of the method, the cancer being treated can be a colon cancer, breast cancer, ovarian cancer, cervical cancer, lung cancer, rectal cancer, kidney cancer, liver cancer, brain cancer, or leukemia, or a combination thereof. In some forms of the method, the cancer can be breast cancer, such as triple negative breast cancer (TNBC).

In some forms of the method, the cancer can be AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system lymphoma, central nervous system lymphoma, cerebellar astrocytoma, brain astrocytoma, cancer of the cervix, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood brain astrocytoma, childhood extracranial germ cell tumors, childhood Hodgkin's disease, childhood Hodgkin's lymphoma, childhood visual pathway and hypothalamic glioma, childhood lymphoblastic leukemia, childhood medulloblastoma, childhood non-Hodgkin's lymphoma, childhood supratentorial primitive neuroectodermal and pineal tumors, childhood primary liver cancer, childhood rhabdomyosarcoma, childhood soft tissue sarcoma, childhood visual pathway and hypothalamic glioma, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the oesophagus, Ewing's sarcoma and related tumors, cancer of the exocrine pancreas, extracranial germ cell tumor, extragonadal germ cell tumor, extrahepatic biliary tract cancer, cancer of the eye, breast cancer in women, Gaucher's disease, cancer of the gallbladder, gastric cancer, gastrointestinal carcinoid tumor, gastrointestinal tumors, germ cell tumors, gestational trophoblastic tumor, tricoleukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, cancer of kidney, cancer of the larynx, cancer of the lip and mouth, cancer of the liver, cancer of the lung, lymphoproliferative disorders, macroglobulinemia, breast cancer in men, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma during pregnancy, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, phaeochromocytoma, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

### 2. Effective Amount/Dosage

Generally, following the adminsitration step of the disclosed method, the pharmaceutical formulation is administered in an effective amount to treat or ameliorate one or more symptoms associated with a cancer in a subject.

In some forms, the pharmaceutical formulation is administered in an effective amount to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment.

Administering an effective amount of the pharmaceutical formualation can be achieved in a single administration step or using multiple administration steps. For example, if the unit dosage form contains an effective amount of the compound(s) to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment, then the method only requires a single administration step. Alternatively, if the unit dosage form contains less than the required effective amount of the compound(s) to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment, then the method involves at least two steps of administering the pharmaceutical formulation, and optionally more than two steps of administering the pharmaceutical formulation to the subject until an effective amount of the pharmaceutical formulation is administered to the subject to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment. When multiple administration steps are needed to administer an effective amount of the pharmaceutical formulation to the patient, each administration step may administer the same dosage or different dosages of the pharmaceutical formulation to the patient.

When multiple administration steps are needed to administer an effective amount of the pharmaceutical formulation to the patient, the administration steps may be performed regularly or irregularly. For example, the administration steps are performed at a suitable frequency, such as every hour, every 2 hours, every 5 hours, every 8 hours, every day, every 2 days, every 3 days, every 5 days, every 7 days, every 10 days, every two weeks, or every month. For example, the administration step is performed every hour, every 2 hours, every 5 hours, every 8 hours, every day, every 2 days, every 3 days, every 5 days, every 7 days, every 10 days, every two weeks, or every month for a period between one day and 6 months, between one day and 5 months, between one day and 4 months, between one day and 3 months, between one and thirty days, between one and ten days, between one and three days, between one and two days, or for one day. Alternatively, the administration may be performed irregularly, for example, the administration step is performed 1 day after the first administration, then 2 days after the second administration, then 5 days after the third administration, then 7 day after the fourth administration, and then 30 days after the fifth administration. The time interval between administrations is determined based on the patient's needs.

In some forms, the method includes only a single administration of the pharmaceutical formulation, wherein the pharmaceutical formulation contains an effective amount of the compounds to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment.

In some forms, the method includes more than one step of administering to the subject the pharmaceutical formulation, wherein following all of the administration steps an effective amount of the compounds to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment, is administered to the subject.

In some forms, following a single administration or multiple administrations, the effective amount of compound(s) that is administered to the subject to treat or ameliorate one or more symptoms associated with a cancer in a subject, such as to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment, can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 20 mg/kg of the subject.

### 3. Optional Steps

a. Administering Additional Active Agent(s)

One or more active agents in addition to the compounds may be administered to the subject throughout the method or at different intervals during the method. For example, the one or more additional active agents is administered to the subject prior to, during, and/or subsequent to step (i). In some forms, the one or more additional active agents can be included in a pharmaceutical formulation containing the compound(s) and is administered to the subject simultaneously with the compound(s) in the pharmaceutical formulation in association with one or more pharmaceutically acceptable excipients. In some forms, the one or more additional active agents can be administered separately from the pharmaceutical formulation containing the compound(s).

In some forms, the one or more additional active agents are one or more anticancer agents described above. The amount of the one or more additional anticancer agents required will vary from subject to subject according to their need.

### B. Treating Cancer Cells

In some forms, the compounds can be used in a method for treating cancer cells in a subject in need thereof.

The method can follow the method step described above, for example, administering to the subject the pharmaceutical formulation containing the comopund(s), such as by oral administration, parenteral administration, inhalation, mucosal, topical administration, or a combination thereof.

The administration step can occur one or more times to administer an effective amount of the compound(s) in the pharmaceutical formulation to induce apoptosis of the cancer cells, depending on whether a unit dosage contains an effective amount of the compound(s) to induce apoptosis of the cancer cells. When multiple administrations are needed to achieve a required effective amount of the compound(s) to induce apoptosis of the cancer cells in the subject, the dosage and frequency for each administration can follow the method described above.

In some forms of the method, following a single administration or multiple administrations, the effective amount of compound(s) that is administered to the subject is effective to downregulate MDM2 and upregulate p53 in test cancer cells compared to the test cancer cells before treatment as shown by Western blot.

In some forms of the method, the compound(s) can have an IC₅₀ value against test cancer cells comparable to or lower than an IC₅₀ value of BW-AQ-101 and/or BW-AQ-238 against the same test cancer cells, tested under the same conditions.

The cancer cells being treated in the subject using the methods disclosed herein can be the cancer cells of any one of the cancers described above. For example, the cancer cells can be cancer cells harboring WT-p53, such as leukemia cells harboring WT-p53, MCF7 cells, RS4;11 cells, or HeLa cells, or a combination thereof. In some forms, the cancer cells being treted in the subject using the methods disclosed herein can be EU-1 leukemia cells.

In some forms, following a single administration or multiple administrations, the effective amount of compound(s) that is administered to the subject to treat cancer cells in a subject, such as to induce apoptosis of the cancer cells in the subject, compared to the subject before administered with the pharmaceutical formulation, can be in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 20 mg/kg of the subject.

In some forms, the method can include the additional step described above. For example, the user can administer one or more additional active agents to the subject prior to, during, and/or subsequent to adminisering the compound to the subject.

The disclosed compounds, methods of using, and methods of making can be further understood through the following enumerated paragraphs.

1. A compound having the structure of

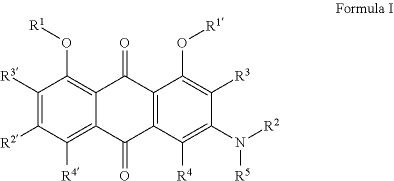

wherein
(a) R¹ and R¹′ are independently a hydrogen, a hydroxyalkyl, or a carbonyl (e.g., a ketone), wherein at least one of R¹ and R¹′ is not hydrogen;
(b) R² is an electrophilic group;
(c) R⁵ is a hydrogen, a substituted or unsubstituted alkyl, a substituted or unsubstituted alkenyl, or a substituted or unsubstituted alkynyl;
(d) R³, R⁴, R²′, R³′, and R⁴′ are independently a hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclyl, a halogen, a hydroxyl, an amino, an amido, an azo, an azido, an alkoxy, a polyether, a thiol, a cyano, a nitro, or a carbonyl;
(e) each substituent is independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an azido, an oxo, a silyl, a sulfinyl, a sulfonyl, a sulfonic acid, a phosphonium, a phosphanyl, a phosphoryl, or a phosphonyl.

2. The compound of paragraph 1, wherein R² is a carbonyl, a sulfinyl, a sulfonyl, or a sulfamoyl.

3. The compound of paragraph 1 or 2, wherein R² is

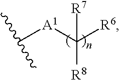

and wherein
A¹ is

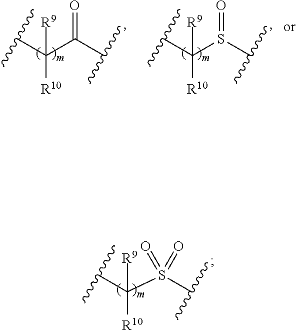

n and m are independently an integer from 0 to 6, from 0 to 5, from 0 to 4, from 0 to 3, or from 0 to 2, such as 1 or 0;
R⁶ is a halogen (e.g., fluorine, chlorine, bromine, or iodine), a cyano, an azido, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, or a substituted or unsubstituted heterocyclyl; and
R⁷—R¹⁰ are independently a hydrogen, a substituted or unsubstituted alkyl, a substituted or unsubstituted alkenyl, a substituted or unsubstituted alkynyl, a halogen, a hydroxyl, an amino, an amido, an alkoxy, an azo, an azido, a polyether, a thiol, a cyano, a nitro, a nitrile, a carbonyl, a sulfinyl, a sulfonyl, or a sulfamoyl.

4. The compound of any one of paragraphs 1-3, wherein R² is

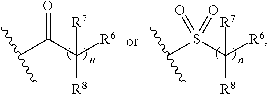

and wherein
n is an integer from 0 to 3, from 0 to 2, 0 or 1;
R⁶ is a halogen (e.g., fluorine, chlorine, bromine, or iodine), a cyano, an azido, a substituted or unsubstituted alkenyl, or a substituted or unsubstituted alkynyl; and
R⁷ and R⁸ are independently a hydrogen, a substituted or unsubstituted alkyl, a substituted or unsubstituted alkenyl, a substituted or unsubstituted alkynyl, a halogen, a cyano, or an azido.

5. The compound of paragraph 4, wherein R⁶ is a halogen (e.g., fluorine, chlorine, bromine, or iodine), an azido, an unsubstituted C₂-C₆ alkenyl, or an unsubstituted C₂-C₆ alkynyl, such as a chlorine, an azido, an unsubstituted ethylenyl, an unsubstituted propylenyl, an unsubstituted ethynyl, or an unsubstituted propynyl.

6. The compound of any one of paragraphs 1-5, wherein R¹ and R¹′ are independently a hydrogen, and wherein:

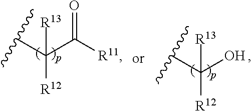

p is an integer from 0 to 6, from 0 to 5, from 0 to 4, from 1 to 5, from 2 to 5, from 1 to 4, or from 2 to 4, such as 3; and
R¹¹_R¹³ are independently a hydrogen, a substituted or unsubstituted alkyl, a substituted or unsubstituted alkenyl, a substituted or unsubstituted alkynyl, a halogen, a hydroxyl, an amino, an amido, an alkoxy, an azo, an azido, a polyether, a thiol, a cyano, a nitro, or a nitrile.

7. The compound of paragraph 6, wherein R¹¹ is a hydrogen, a hydroxyl, or a substituted or unsubstituted alkyl; and R¹² and R¹³ are independently a hydrogen or a substituted or unsubstituted alkyl.

8. The compound of paragraph 6 or 7, wherein R¹¹ is an unsubstituted C₁-C₆ alkyl, an unsubstituted C₁-C₅ alkyl, an unsubstituted C₁-C₄ alkyl, an unsubstituted C₁-C₃ alkyl, an unsubstituted methyl or ethyl, such as methyl.

9. The compound of any one of paragraphs 6-8, wherein R¹² and R¹³ are hydrogen.

10. The compound of any one of paragraphs 1-9, wherein R⁵ is hydrogen.

11. The compound of any one of paragraphs 1-10, wherein R³, R⁴, R²′, R³′, and R⁴′ are independently a hydrogen or a substituted or unsubstituted alkyl.

12. The compound of any one of paragraphs 1-11, wherein R³, R⁴, R²′, R³′, and R⁴′ are hydrogen.

13. The compound of any one of paragraphs 1-12, wherein the compound has the structure of:

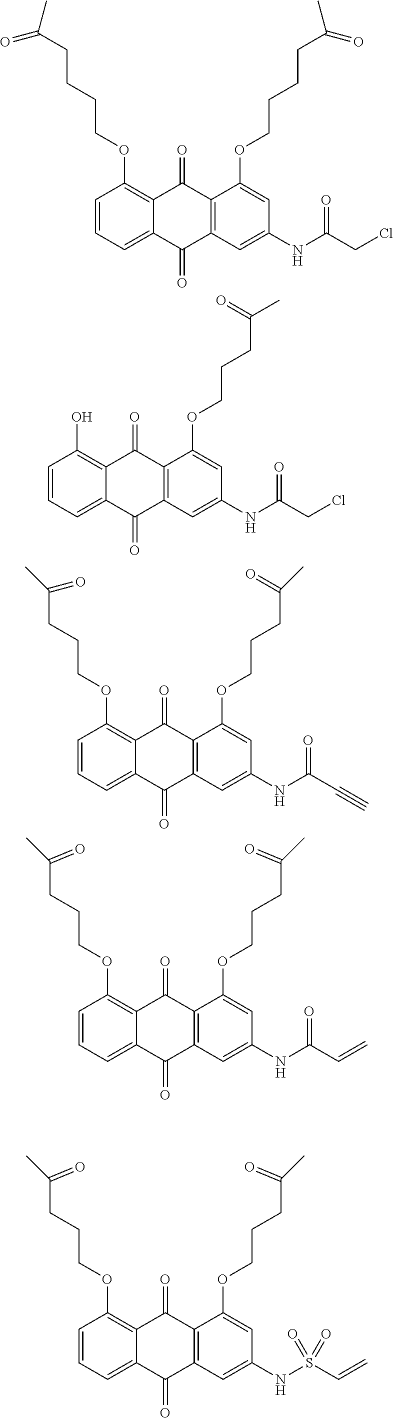

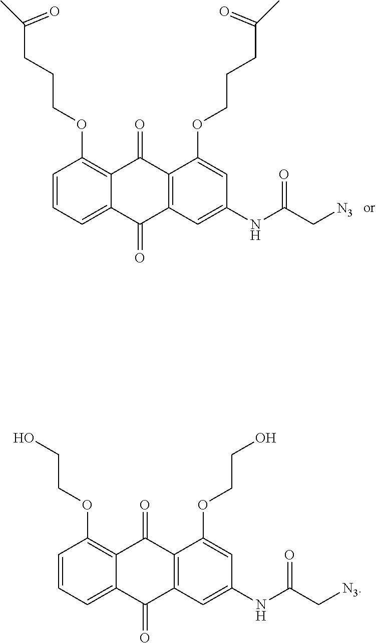 [SMILES: CC(=O)CCCOc1cccc2c1C(=O)c1c(OCCCC(C)=O)cc(NC(=O)CN=[N+]=[N-])cc1C2=O ; [N-]=[N+]=NCC(=O)Nc1cc(OCCO)c2c(c1)C(=O)c1cccc(OCCO)c1C2=O]

14. The compound of any one of paragraphs 1-12, wherein the compound is not

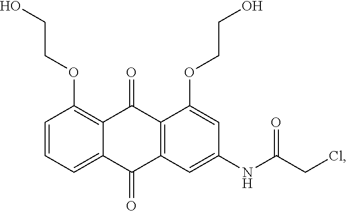 [SMILES: O=C(CCl)Nc1cc(OCCO)c2c(c1)C(=O)c1cccc(OCCO)c1C2=O]

not

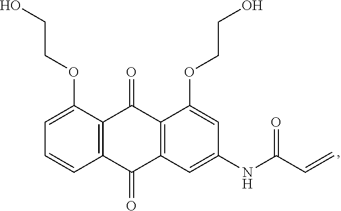 [SMILES: C=CC(=O)Nc1cc(OCCO)c2c(c1)C(=O)c1cccc(OCCO)c1C2=O]

not

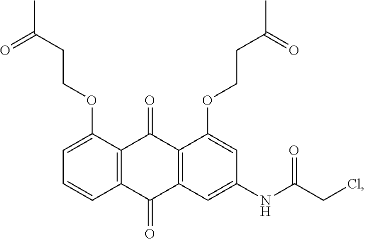 [SMILES: CC(=O)CCOc1cccc2c1C(=O)c1c(OCCC(C)=O)cc(NC(=O)CCl)cc1C2=O]

and not

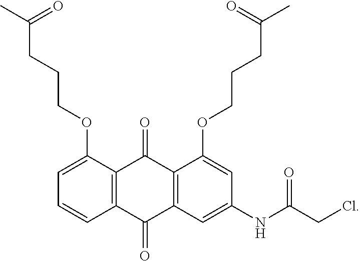 [SMILES: CC(=O)CCCOc1cccc2c1C(=O)c1c(OCCCC(C)=O)cc(NC(=O)CCl)cc1C2=O]

15. The compound of any one of paragraphs 1-14, wherein the compound has an IC₅₀ value against test cancer cells comparable or lower than an IC₅₀ value of BW-AQ-101 and/or BW-AQ-238 against the same test cancer cells, tested under the same conditions.

16. A pharmaceutical formulation comprising
one or more compounds of any one of paragraphs 1-15; and
a pharmaceutically acceptable carrier and/or excipient.

17. The pharmaceutical formulation of paragraph 16, wherein the one or more compounds are in an effective amount to treat or ameliorate one or more symptoms associated with a cancer in a subject.

18. The pharmaceutical formulation of paragraph 17, wherein the effective amount of the one more compounds is effective to induce apoptosis of cancer cells in a subject.

19. The pharmaceutical formulation of paragraph 18, wherein the cancer cells are cancer cells harboring WT-p53, and therein the effective amount of the one or more compounds is effective to downregulate MDM2 and upregulate p53 in the cancer cells compared to the cancer cells before treatment as shown by Western blot.

20. The pharmaceutical formulation of paragraph 18 or 19, wherein the cancer cells are leukemia cells harboring WT-p53 (e.g., EU-1 leukemia cells), MCF7 cells, RS4;11 cells, or HeLa cells, or a combination thereof.

21. The pharmaceutical formulation of any one of paragraphs 16-20, wherein the pharmaceutical formulation further comprises a second active agent, optionally more than one second active agent, optionally wherein the second active agent is an anticancer agent.

22. The pharmaceutical formulation of any one of paragraphs 16-21, wherein the total concentration of the one or more compounds in the pharmaceutical formulation is at least 0.001 wt %, at least 0.005 wt %, at least 0.01 wt %, at least 0.05 wt %, at least 0.1 wt %, in a range from 0.01 wt % to 50 wt %, from 0.05 wt % to 50 wt %, from 0.1 wt % to 50 wt %, from 0.01 wt % to 40 wt %, from 0.05 wt % to 40 wt %, from 0.1 wt % to 40 wt %, from 0.01 wt % to 30 wt %, from 0.05 wt % to 30 wt %, from 0.1 wt % to 30 wt %, from 0.01 wt % to 20 w1%, from 0.05 w1% to 20 w1%, from 0.1 wt % to 20 wt %, from 0.01 wt % to 10 wt %, from 0.05 wt % to 10 wt %, or from 0.1 wt % to 10 wt %.

23. A method for treating or ameliorating one or more symptoms associated with a cancer in a subject in need thereof comprising
(i) administering to the subject the pharmaceutical formulation of any one of paragraphs 16-22, wherein step (i) occurs one or more times.

24. The method of paragraph 23, wherein the cancer is leukemia, such as Acute lymphocytic leukemia (ALL), Acute myelogenous leukemia (AML), Chronic lymphocytic leukemia (CLL), or Chronic myelogenous leukemia (CML).

25. The method of paragraph 24, wherein the method comprises only a single administration of the pharmaceutical formulation, wherein the pharmaceutical formulation comprises an effective amount of the compounds to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment.

26. The method of paragraph 24, wherein the method comprises more than one step of administering to the subject the pharmaceutical formulation, wherein following all of the administration steps an effective amount of the compounds to reduce the number of leukemia cells in the blood of the subject by at least 20%, at least 30%, at least 40%, at least 50%, at least 55%, at least 60%, at least 65%, at least 70%, at least 75%, at least 80%, at least 85%, or at least 90% in the subject compared to the number of leukemia cells in the blood of the subject before treatment, is administered to the subject.

27. The method of paragraph 26, wherein the administration is performed every day, every 2 days, every 3 days, every 5 days, every 7 days, every 10 days, every two weeks, or every month for a period from one day to 6 months, from one day to 5 months, from one day to 4 months, from one day to 3 months, from one day to 2 months, from one day to thirty days, or from one day to ten days.

28. The method of any one of paragraphs 25-27, wherein the effective amount of the compounds is in a range from about 0.1 mg/kg to about 50 mg/kg, in a range from about 0.3 mg/kg to about 30 mg/kg, in a range from about 0.5 mg/kg to about 20 mg/kg, in a range from about 1 mg/kg to about 15 mg/kg, or in a range from about 0.5 mg/kg to about 10 mg/kg, such as about 20 mg/kg.

29. The method of any one of paragraphs 23-28, wherein the subject is a mammal.

30. The method of any one of paragraphs 23-29, wherein the pharmaceutical formulation is administered by oral administration, parenteral administration, inhalation, mucosal administration, topical or a combination thereof.

31. The method of any one of paragraphs 23-30, wherein the method further comprises administering to the subject a second active agent, optionally more than one second active agent, prior to, during, and/or subsequent to step (i).

32. The method of paragraph 31, wherein the second active agent is an anticancer agent.

33. A method for treating cancer cells in a subject in need thereof comprising (i) administering to the subject the pharmaceutical formulation of any one of paragraphs 16-22, wherein step (i) occurs one or more times.

34. The method of paragraph 33, wherein the method comprises only a single administration of the pharmaceutical formulation, wherein the pharmaceutical formulation comprises an effective amount of the compounds to induce apoptosis of the cancer cells.

35. The method of paragraph 33, wherein the method comprises more than one step of administering to the subject the pharmaceutical formulation, wherein following all of the administration steps an effective amount of the compounds to induce apoptosis of the cancer cells is administered to the subject.

36. The method of paragraph 34 or 35, wherein the effective amount of the compounds is effective to downregulate MDM2 and upregulate p53 in test cancer cells compared to the test cancer cells before treatment as shown by Western blot.

37. The method of any one of paragraphs 34-36, wherein the compound has an IC₅₀ value against test cancer cells comparable to or lower than an IC₅₀ value of BW-AQ-101 and/or BW-AQ-238 against the same test cancer cells, tested under the same conditions.

38. The method of any one of paragraphs 34-37, wherein the cancer cells are cancer cells harboring WT-p53.

39. The method of paragraph 38, wherein the cancer cells are leukemia cells harboring WT-p53, MCF7 cells, RS4;11 cells, or HeLa cells, or a combination thereof.

40. The method of paragraph 38 or 39, wherein the cancer cells are EU-1 leukemia cells.

The present invention will be further understood by reference to the following non-limiting examples.

### Examples

### Example 1. Introduction of an Alkylketone Sidechain on the Anthraquinone Core Improves Compound Potency Against Cancer Cells

### Materials and Methods

### Cell Culture and Cytotoxicity Test

Cell viability was assessed by using Cell Counting Kit-8 (CCK-8, Dojindo, Japan). EU-1 and RS4;11 cells were cultured in RPMI-1640 medium (Corning, USA); MCF7 and HEK-293 cells were cultured in MEM (Corning, USA); and HeLa and H₉c2 cells were cultured in high glucose DMEM (Corning, USA). All the culture mediums were supplemented with 10% fetal bovine serum (Corning, USA) and 100 unit/ml penicillin, and 0.1 μg/ml streptomycin. For cytotoxicity assays, cells were seeded into 96-well plates at a density of 3× 10⁵ cells per well (50 μL). Compounds were dissolved in DMSO (molecular biology grade, Sigma-Aldrich, USA) to make 10 mM stock solutions, which were serially diluted with the culture medium to various concentrations (final DMSO concentration=0.5%). A culture medium (50 μL) containing the anthraquinone analog was added directly to the cell suspension in each well immediately after seeding. After incubation for 24 h at 37° C. in a humidified atmosphere with 5% CO₂, 10 μL of CCK-8 solution was added to each well, and the plate was incubated for an additional 2-4 h at 37° C. before measuring the optical density at 450 nm with a microplate reader (PerkinElmer Victor 2, USA). The cell viability of each well was calculated as the percentage of the untreated control according to the manufacturer's manual. All tests were performed in triplicates, and IC₅₀ values were determined with six concentrations by non-linear regression using GraphPad Prism 9.

### Cell Culture and Cytotoxicity Test of BW-AQ-365

Cell viability was assessed by using Cell Counting Kit-8 (CCK-8, Dojindo, Japan). RS4;11 cells (wild-type p53) were cultured in RPMI-1640 medium (Corning, USA). The culture mediums were supplemented with 10% fetal bovine serum (Corning, USA) and 100 unit/ml penicillin, and 0.1 μg/ml streptomycin. For cytotoxicity assays, cells were seeded into 96-well plates at a density of 1.0~2.5×10⁵ cells per well (50 μL). Compounds were dissolved in DMSO (molecular biology grade, Sigma-Aldrich, USA) to make 10 mM stock solutions, which were serially diluted with the culture medium to various concentrations (final DMSO concentration=0.5%). A culture medium (50 μL) containing BW-AQ-365 was added directly to the cell suspension in each well immediately after seeding. After incubation for 24-72 h at 37° C. in a humidified atmosphere with 5% CO₂, 10 μL of CCK-8 solution was added to each well, and the plate was incubated for an additional 2-4 h at 37° C. before measuring the optical density at 450 nm with a microplate reader (PerkinElmer Victor 2, USA). The cell viability of each well was calculated as the percentage of the untreated control according to the manufacturer's manual. All tests were performed in triplicates, and IC₅₀ values were determined by non-linear regression using GraphPad Prism 9.0.

Western Blot 5×10⁶ cells were seeded in a 6-well plate and incubated for 12 h before the drug-loaded medium was added. The cells were harvested at designated time points, washed with cold PBS, and centrifuged at 1500 rpm×3 min. Then the cells were lysed by adding 100 μL of cold NP-40 buffer (supplied with complete™ protease Inhibitor tablet (Roche, USA)) and 1 mM PMSF (Thermo-Fisher, USA) on ice for 30 min. The cell lysates were centrifuged at 12500×g at 4° C. for 10 min, and the total protein concentration in the supernatant was measured with the BCA assay (Thermo-Fisher, USA). 30 μL of the cell lysate was mixed with 10 μL 4×Laemmli sample buffer and denatured at 95° C. for 5 min. The total protein concentration was adjusted with 1×Laemmli sample buffer. Equal amounts of protein sample were loaded onto a 4-15% gradient SDS-PAGE gel (Bio-rad, USA). After electrophoresis, the protein was transferred to the PTFE membrane (Bio-rad, USA) with the Trans-Blot Turbo system (Bio-rad, USA). The target protein was probed with the corresponding antibody using iBind Western Systems (Thermo-Fisher, USA), according to the manufacturer's manual. Antibody and dilution profile: MDM2 ((SMP14), 1:500 Santa Cruz, USA); p53 ((DO-1), 1:800, Santa Cruz, USA); GAPDH ((0411), 1:2000, Santa Cruz, USA) and HRP conjugated goat anti-mouse secondary antibody (1:2000, Bio-rad, USA). After incubation with Pierce ECL Plus Substrate (Thermo Scientific), chemiluminescent was detected and imaged with LSA⁴⁰⁰⁰ (GE Healthcare, Fairfield, USA).

### Reactivity Study by HPLC

HPLC was performed on Shimadzu LC-20AT HPLC system. Condition: mobile phase A: water with 0.05% TFA; B: acetonitrile with 0.05% TFA; gradient: 5-95% B, 0-10 min; 5-95% B 10-12 min; 5% B 12.1-15 min. Column: C18, 3.5 μm, 4.6×150 mm; UV detector monitored at 254 nm. For the thiol reactivity assay, a 0.3 mM solution of AQ-analogs was prepared in 0.5 mL PBS: MeOH (80:20) and warmed at 37° C. Next, 25 μL of NAC (from 30 mM stock solution in PBS) was added to the above solution. The resultant solution had final concentrations of 0.3 mM AQ-analogs and 1.5 mM NAC. 20 μL of the reaction mixture was injected into the HPLC at the designated time intervals. The area percentage values were determined via the integration of the area under the curve (AUC) of the chromatogram.

### Synthesis and Characterization of the Compounds

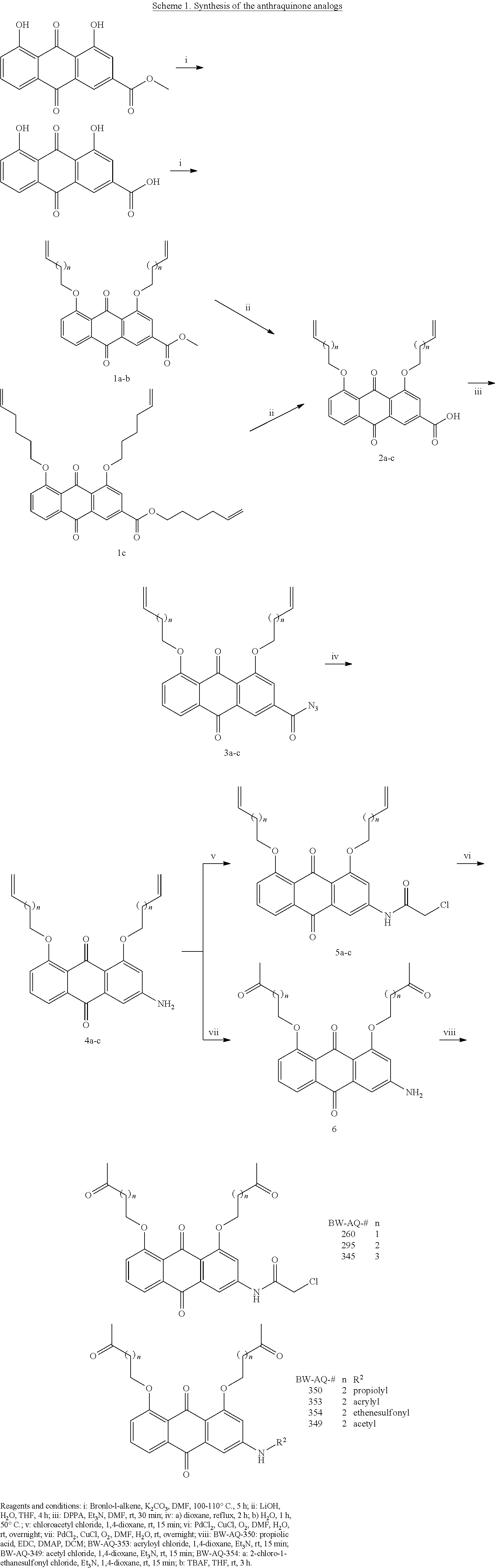

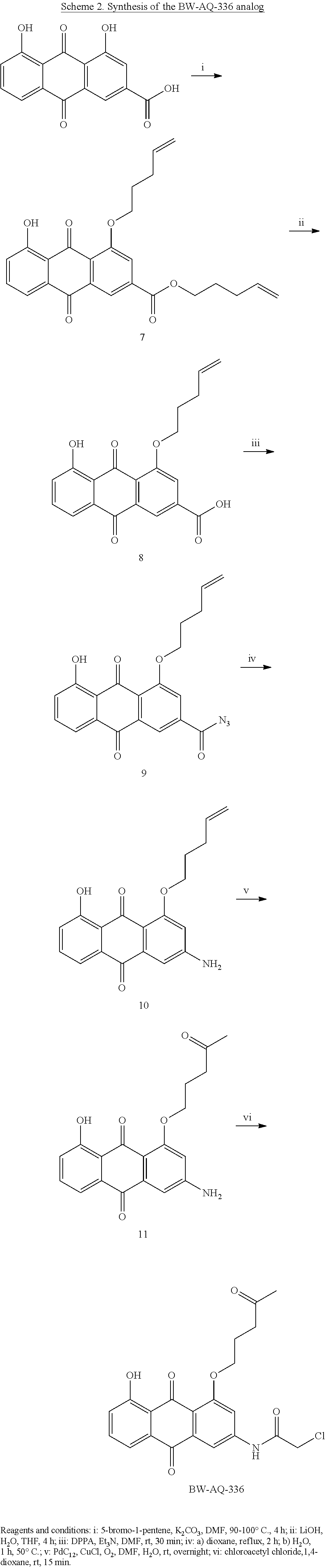

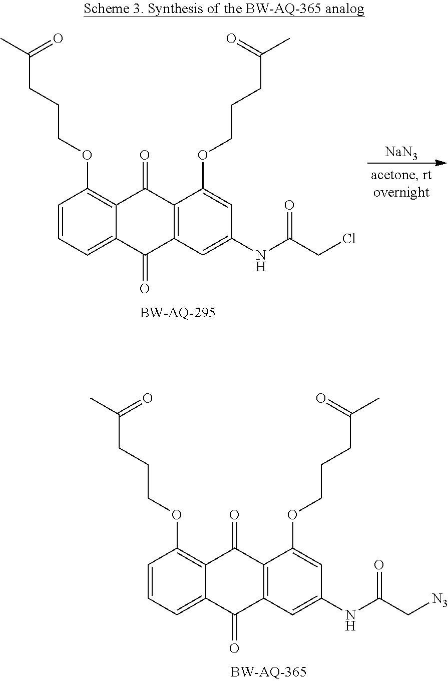 [SMILES: CC(=O)CCCOc1cccc2c1C(=O)c1c(OCCCC(C)=O)cc(NC(=O)CCl)cc1C2=O ; CC(=O)CCCOc1cccc2c1C(=O)c1c(OCCCC(C)=O)cc(NC(=O)CN=[N+]=[N-])cc1C2=O ; "NaN3"]

### General Information

Rhein was purchased from Nanjing Zelang Medical Technology Co. Ltd. (P.R. China) and used without further purification. All other starting materials were purchased from Sigma-Aldrich (USA) or Oakwood Chemical (USA), and analytical-grade solvents were purchased from Fisher Scientific (USA). Anhydrous chemical solvents were used for all moisture-sensitive reactions. High-resolution mass spectrometry (HRMS) analyses were conducted by the Georgia State University mass spectrometry facilities using ABI API 3200 (ESI-Triple Quadruple) instrument. 1H-(400 MHz) and ¹³C-NMR (100 MHz) spectra were recorded on a Bruker Avance 400 MHz NMR spectrometer. Deuterated solvents were purchased from Cambridge Isotope Laboratories, Inc (USA). Chemical shifts were reported as 8 values (ppm). TMS (8=0.00 ppm) or residual peaks of the deuterated solvent were used as the internal reference.

### General Procedure for the Synthesis of Compounds 1 and 7

Rhein (for compounds 1c and 7) or its methyl ester (for compound 1a-b) (1.676 mmol) was suspended in DMF (40 mL) in a dry round bottom flask. Next, K₂CO₃ (8.38 mmol) was added to the mixture, and the mixture was stirred gently for 5 min till the complete dissolution of all components. Further, an alkylating agent (for disubstituted-8.38 mmol; for mono-substituted 4.19 mmol) was added gradually. The solution was stirred for 4-5 h at 90-100° C. After consumption of the starting material, according to TLC (DCM: methanol=10:1), the reaction mixture was transferred to a conical flask having 200 mL water. The compound was extracted by DCM, washed by 1 M HCl and brine, and dried in-vacuo to yield a yellow solid product in good yield and purity. methyl 4,5-bis(but-3-en-1-yloxy)-9,10-dioxo-9,10-dihydroanthracene-2-carboxylate (1a): Isolated yield: 86%, ¹H-NMR (Chloroform-d) δ 8.45 (d, J=1.6 Hz, 1H), 7.92 (d, J=1.6 Hz, 1H), 7.87-7.83 (m, 1H), 7.63 (t, J=8.0 Hz, 1H), 7.33-7.29 (m, 1H), 6.02-6.07 (m, 2H), 5.24-5.30 (m, 2H), 5.14-5.20 (m, 2H), 4.26 (t, J=6.6 Hz, 2H), 4.20 (t, J=6.7 Hz, 2H), 4.00 (s, 3H), 2.71 (t, J=6.9 Hz, 4H). ¹³C-NMR (Chloroform-d) δ 183.3, 181.5, 165.5, 158.7, 134.9, 134.6, 134.5, 134.1, 134.0, 133.9, 127.4, 124.6, 119.9, 119.9, 119.8, 119.6, 119.2, 117.5, 117.4, 69.4, 69.4, 69.2, 52.7, 33.6, 33.5. HRMS (ESI) m/z: Calculated for C₂₄H₂₃₀₆[M+H]⁺407.1496; Found 407.1495. methyl 9,10-dioxo-4,5-bis(pent-4-en-1-yloxy)-9,10-dihydroanthracene-2-carboxylate (1b): Isolated yield: 91%, ¹H-NMR (Chloroform-d) δ 8.45 (s, 1H), 7.92 (s, 1H), 7.84 (d, J=7.7 Hz, 1H), 7.63 (t, J=8.0 Hz, 1H), 7.31 (d, J=8.3 Hz, 1H), 5.99-5.84 (m, 2H), 5.16-5.07 (m, 2H), 5.02 (d, J=10.0 Hz, 2H), 4.23 (t, J=6.3 Hz, 2H), 4.17 (t, J=6.3 Hz, 2H), 4.00 (s, 3H), 2.40 (dq, J=10.7, 6.6 Hz, 4H), 2.04 (h, J=6.8 Hz, 4H). ¹³C-NMR (Chloroform-d) δ 183.4, 181.6, 165.6, 158.8, 137.8, 137.7, 134.9, 134.7, 134.4, 133.8, 127.4, 124.5, 119.7, 119.6, 119.4, 119.0, 115.3, 115.3, 69.1, 68.8, 52.7, 29.9, 29.9, 28.2, 28.2. HRMS (ESI) m/z: Calculated for C₂₆H₂₆₀₆Na[M+Na]⁺457.1611; Found 457.1627.

hex-5-en-1-yl 4,5-bis(hex-5-en-1-yloxy)-9,10-dioxo-9,10-dihydroanthracene-2-carboxylate (1c): Isolated yield: 78%, ¹H-NMR (Chloroform-d) δ 8.42 (s, 1H), 7.90 (s, 1H), 7.82 (d, J=7.5 Hz, 1H), 7.61 (t, J=8.0 Hz, 1H), 7.29 (d, J=8.3 Hz, 1H), 5.94-5.76 (m, 3H), 5.02 (dd, J=31.0, 13.5 Hz, 6H), 4.38 (t, J=6.6 Hz, 2H), 4.20 (t, J=6.3 Hz, 2H), 4.14 (t, J=6.2 Hz, 2H), 2.23-2.10 (m, 6H), 1.93 (dt, J=13.0, 6.4 Hz, 4H), 1.87-1.78 (m, 2H), 1.75-1.63 (m, 4H), 1.56 (dt, J=14.4, 7.3 Hz, 2H). ¹³C-NMR (Chloroform-d) 8 183.7, 181.7, 165.3, 159, 138.7, 138.7, 138.3, 135, 134.9, 134.8, 134, 127.4, 124.6, 119.7, 119.6, 119.5, 119, 115.1, 114.9, 114.9, 69.9, 69.6, 65.9, 33.5, 33.5, 33.4, 28.6, 28.5, 28.2, 25.3, 25.3, 25.2. HRMS (ESI) m/z: Calculated for C₃₃H₃₉₀₆[M+H]⁺531.27; Found 531.2749.

pent-4-en-1-yl 5-hydroxy-9,10-dioxo-4-(pent-4-en-1-yloxy)-9,10-dihydroanthracene-2-carboxylate (7): Isolated yield: 41%, ¹H-NMR (Chloroform-d) δ 12.89 (s, 1H), 8.48 (d, J=1.2 Hz, 1H), 7.92 (d, J=0.9 Hz, 1H), 7.76 (d, J=7.5 Hz, 1H), 7.62 (t, J=7.9 Hz, 1H), 7.29 (d, J=8.3 Hz, 1H), 5.96-5.80 (m, 2H), 5.15-5.00 (m, 4H), 4.40 (t, J=6.6 Hz, 2H), 4.24 (t, J=6.3 Hz, 2H), 2.40 (q, J=7.0 Hz, 2H), 2.24 (q, J=7.0 Hz, 2H), 2.10-2.01 (m, 2H), 1.97-1.88 (m, 2H). ¹³C-NMR (Chloroform-d) δ 188.1, 182.1, 164.9, 162.6, 160.5, 137.5, 137.3, 136.5, 136.1, 135.9, 132.6, 125, 123.4, 120.4, 119.5, 119.1, 117.1, 115.7, 115.7, 69.2, 65.6, 30.2, 30, 28.2, 27.8. HRMS (ESI) m/z: Calculated for C₂₅H₂₅₀₆[M+H]⁺421.17; Found 421.1661.

### General Procedure for the Synthesis of Compounds 2 and 8

Compound 1 (for compound 2) or 7 (for compound 8) (0.257 mmol) was added to a mixture of THF and KOH (0.309 mmol) in water, and the mixture was stirred at room temperature. After completion of the reaction, monitored by TLC (hexane:ethyl acetate=3:1), THF was removed in-vacuo and residue was neutralized with 1% HCl and extracted by DCM. The solvent was reduced completely in-vacuo to afford the shiny-yellow product.

### 4,5-bis(but-3-en-1-yloxy)-9,10-dioxo-9,10-dihydroanthracene-2-carboxylic acid

(2a): Isolated yield: 93%, ¹H-NMR (DMSO-d6) δ 8.18 (d, J=1.5 Hz, 1H), 7.94-7.87 (m, 1H), 7.83-7.68 (m, 2H), 7.57 (dt, J=8.1, 2.4 Hz, 1H), 6.02-6.07 (m, 2H), 5.21-5.27 (m, 2H), 5.08-5.14 (m, 2H), 4.25 (1, J=6.5 Hz, 2H), 4.19 (t, J=6.5 Hz, 2H), 2.54-2.60 (m, 4H). ¹³C-NMR (DMSO-d₆) δ 183.1, 181.0, 166.3, 158.5, 158.5, 135.8, 135.2, 135.1, 134.9, 134.7, 134.4, 126.8, 124.1, 120.8, 119.9, 119.1, 118.8, 117.7, 117.6, 69.1, 68.9, 33.5, 33.4. HRMS (ESI) m/z: Calculated for C₂₃H₁₉₀₆[M−H]⁻391.1195; Found 391.1182.

### 9,10-dioxo-4,5-bis(pent-4-en-1-yloxy)-9,10-dihydroanthracene-2-carboxylic acid

(2b): Isolated yield: 97%, ¹H-NMR (DMSO-d₆) δ 8.22-8.15 (m, 1H), 7.94-7.86 (m, 1H), 7.81-7.67 (m, 2H), 7.55 (dd, J=8.2, 1.5 Hz, 1H), 5.87-5.95 (m, 2H), 5.10 (q, J=1.9 Hz, 1H), 5.06 (p, J=1.8 Hz, 1H), 4.99 (dt, J=10.2, 1.8 Hz, 2H), 4.18 (dt, J=21.9, 6.1 Hz, 4H), 2.39-2.28 (m, 4H), 1.95-1.82 (m, 4H). ¹³C-NMR (DMSO-d₆) δ 183.3, 181.1, 166.8, 158.5, 138.5, 134.6, 134.4, 134.4, 126.0, 124.0, 120.3, 119.7, 119.0, 118.6, 115.5, 68.7, 68.6, 29.9, 29.9, 28.2, 28.2. HRMS (ESI) m/z: Calculated for C₂₅H₁₃₀₆[M−H]419.1489; Found 419.1495.

### 4,5-bis(hex-5-en-1-yloxy)-9,10-dioxo-9,10-dihydroanthracene-2-carboxylic acid

(2c): Isolated yield: 91%, ¹H-NMR (Chloroform-d) § 8.50 (s, 1H), 7.94 (s, 1H), 7.82 (d, J=7.6 Hz, 1H), 7.61 (t, J=8.0 Hz, 1H), 7.28 (d, J=8.4 Hz, 1H), 5.85 (tddd, J=13.9, 10.6, 6.9, 3.7 Hz, 2H), 5.11-5.06 (m, 1H), 5.06-5.01 (m, 1H), 4.98 (d, J=10.1 Hz, 2H), 4.22 (t, J=6.3 Hz, 2H), 4.13 (t, J=6.3 Hz, 2H), 2.18 (p, J=6.7 Hz, 4H), 2.01-1.88 (m, 4H), 1.70 (dq, J=15.2, 7.8 Hz, 4H). ¹³C-NMR (Chloroform-d) δ 183.4, 181.8, 169.9, 159, 159, 138.6, 138.6, 135.1, 134.7, 134.1, 133.8, 127.9, 124.4, 120.4, 119.7, 119.6, 119.1, 114.9, 114.9, 100.1, 69.9, 69.6, 33.4, 33.4, 28.5, 28.5, 25.2, 25.2. HRMS (ESI) m/z: Calculated for C₂₇H₂₉O₆[M−H]⁻447.515; Found 447.0411.

5-hydroxy-9,10-dioxo-4-(pent-4-en-1-yloxy)-9,10-dihydroanthracene-2-carboxylic acid (8): Isolated yield: 97%, ¹H-NMR (Chloroform-d) δ 12.83 (s, 1H), 8.60 (s, 1H), 8.00 (s, 1H), 7.80 (d, J=7.2 Hz, 1H), 7.64 (t, J=7.8 Hz, 1H), 7.32 (d, J=8.3 Hz, 1H), 5.92 (d, J=6.4 Hz, 1H), 5.08 (dd, J=27.6, 13.3 Hz, 2H), 4.28 (s, 2H), 2.42 (d, J=6.9 Hz, 2H), 2.07 (d, J=5.8 Hz, 2H). ¹³C-NMR (DMSO-d₆) δ 187.6, 181.6, 165.7, 161.4, 160, 138, 137.4, 136.4, 135.2, 132.3, 124.4, 122.4, 119.3, 119.2, 118.4, 116.9, 115.3, 68.6, 40.1, 39.9, 39.1, 38.8, 29.5, 27.6. HRMS (ESI) m/z: Calculated for C₂₀H₁₇₀₆[M+H]⁺353.10; Found 353.1014.

### General Procedure for the Synthesis of Compounds 3 and 9

Compound 2 (for compound 3) or 8 (for compound 9) (0.05 mmol) was dissolved in DMF (4 mL). Further, Et₃N (0.118 mmol) was added to the reaction solution. The solution was stirred at 0° C. for 5 min. Next, DPPA (0.113 mmol) was added, and the solution was stirred continuously at room temperature till starting material was consumed. After completion of the reaction, according to TLC (DCM: methanol=10:1), about 10 mL of water was added to the mixture, and the light-yellow precipitate was obtained. The precipitate was filtered and washed generously with water.

4,5-bis(but-3-en-1-yloxy)-9,10-dioxo-9,10-dihydroanthracene-2-carbonyl azide (3a): Isolated yield: 89%, ¹H-NMR (Chloroform-d) δ 8.55 (s, 1H), 7.98 (s, 1H), 7.87 (d, J=7.6 Hz, 1H), 7.66 (t, J=8.0 Hz, 1H), 7.33 (d, J=8.5 Hz, 1H), 6.04-6.11 (m, 2H), 5.35-5.22 (m, 2H), 5.23-5.12 (m, 2H), 4.29 (t, J=6.6 Hz, 2H), 4.22 (t, J=6.7 Hz, 2H), 2.73 (p, J=7.3 Hz, 4H). ¹³C-NMR (Chloroform-d) δ 183.2, 181.5, 168.8, 158.7, 158.7, 135.1, 134.6, 134.1, 134.0, 133.4, 128.0, 124.6, 120.6, 119.9, 119.8, 119.2, 117.6, 117.5, 69.5, 69.2, 33.6, 33.5. HRMS (ESI) m/z: Calculated for C₂₃H₂₀N₃O₅[M+H]⁺418.1300; Found 418.1300.

9,10-dioxo-4,5-bis(pent-4-en-1-yloxy)-9,10-dihydroanthracene-2-carbonyl azide (3b): Isolated yield: 95%, ¹H-NMR (Chloroform-d) δ 8.41 (d, J=1.6 Hz, 1H), 7.88 (d, J=1.6 Hz, 1H), 7.83 (d, J=7.6 Hz, 1H), 7.64 (t, J=8.0 Hz, 1H), 7.31 (d, J=8.3 Hz, 1H), 5.98-5.83 (m, 2H), 5.07-5.15 (m, 2H), 4.99-5.04 (m, 2H), 4.19 (dt, J=21.8, 6.3 Hz, 4H), 2.40 (p, J=6.7 Hz, 4H), 2.05 (dp, J=14.2, 6.7 Hz, 4H). ¹³C-NMR (Chloroform-d) δ 183.1, 181.3, 171.3, 158.9, 158.8, 137.7, 137.6, 135.1, 134.6, 134.5, 134.0, 128.2, 124.4, 119.6, 119.6, 119.0, 118.6, 115.4, 115.3, 69.1, 68.8, 29.9, 29.9, 28.2, 28.1. HRMS (ESI) m/z: Calculated for C₂₅H₂₃N₃O₅Na[M+Na]⁺468.1530; Found 468.1535.

4,5-bis(hex-5-en-1-yloxy)-9,10-dioxo-9,10-dihydroanthracene-2-carbonyl azide (3c): Isolated yield: 98%, ¹H-NMR (Chloroform-d) § 8.39 (d, J=1.2 Hz, 1H), 7.85 (d, J=1.0 Hz, 1H), 7.81 (d, J=7.6 Hz, 1H), 7.61 (t, J=8.0 Hz, 1H), 7.28 (d, J=8.3 Hz, 1H), 5.92-5.78 (m, 2H), 5.11-5.02 (m, 2H), 4.98 (d, J=10.2 Hz, 2H), 4.19 (t, J=6.4 Hz., 2H), 4.13 (t, J=6.4 Hz, 2H), 2.16 (t, J=7.0 Hz, 4H), 1.99-1.87 (m, 4H), 1.75-1.63 (m, 4H). ¹³C-NMR (Chloroform-d) δ 183.2, 181.4, 171.4, 159.1, 159, 138.6, 138.6, 135.2, 134.7, 134.6, 134.1, 128.2, 124.4, 119.7, 119.6, 119, 118.6, 114.9, 114.9, 69.9, 69.6, 33.4, 33.4, 28.5, 28.4, 25.2, 25.2. HRMS (ESI) m/z: Calculated for C₂₇H₂₈N₃O₅[M+H]⁺474.20; Found 474.2030.

5-hydroxy-9,10-dioxo-4-(pent-4-en-1-yloxy)-9,10-dihydroanthracene-2-carbonyl azide (9): Isolated yield: 99%, ¹H-NMR (Chloroform-d)) § 12.86 (s, 1H), 8.51 (d, J=1.0 Hz, 1H), 7.93 (s, 1H), 7.80 (d, J=7.5 Hz, 1H), 7.65 (t, J=7.9 Hz, 1H), 7.32 (d, J=8.4 Hz, 1H), 5.90 (ddt, J=17.0, 10.2, 6.7 Hz, 1H), 5.08 (dd, J=25.9, 13.6 Hz, 2H), 4.26 (t, J=6.3 Hz, 2H), 2.41 (q, J=7.0 Hz, 2H), 2.08 (dd, J=13.9, 6.7 Hz, 2H). ¹³C-NMR (Chloroform-d) δ 188, 1, 171.2, 162.7, 160.7, 137.5, 136.3, 136.2, 132.5, 125.2, 124.4, 120.4, 120.1, 119.3, 118.7, 117.2, 115.8, 69.4, 30, 28.2. HRMS (ESI) m/z: Calculated for C₂₀H₁₆N₃O₅[M+H]⁺378.11; Found 378.1095.

### General Procedure for the Synthesis of Compounds 4 and 10

Compound 3 (for compound 4) or 9 (for compound 10) (0.05 mmol) was refluxed in dry dioxane (3.5 mL) for 1 h under an argon atmosphere at a temperature of 130° C. TLC analysis TLC (hexane: ethylacetate=2:1) showed completion of the reaction. Next, the reaction mixture was diluted with water (10 mL) and heated at 50° C. for another 30 min and then cooled to room temperature. The precipitate was filtered off and dried in-vacuo to afford a deep-red solid product.

3-amino-1,8-bis(but-3-en-1-yloxy) anthracene-9,10-dione (4a): Isolated yield: 59%. ¹H-NMR (Chloroform-d) δ 7.78 (dt, J=7.7, 1.6 Hz, 1H), 7.53 (td, J=8.0, 2.8 Hz, 1H), 7.31-7.22 (m, 1H), 7.07 (d, J=2.2 Hz, 1H), 6.48 (d, J=2.2 Hz, 1H), 5.94-6.07 (m, 2H), 5.17-5.25 (m, 2H), 5.07-5.14 (m, 2H), 4.42 (s, 2H), 4.15 (t, J=6.8 Hz, 2H), 4.04 (t, J=6.8 Hz, 2H), 2.72-2.58 (m, 4H). ¹³C-NMR (Chloroform-d) δ 184.5, 180.9, 161.2, 158.7, 151.3, 136.2, 134.8, 134.3, 134.3, 134.3, 133.0, 132.9, 129.4, 124.8, 120.4, 119.2, 117.2, 117.2, 115.9, 105.1, 104.5, 69.4, 69.4, 69.0, 33.6, 33.6, 33.5. HRMS (ESI) m/z: Calculated for C₂₂H₂₂NO₄Na[M+Na]⁺386.1352; Found 386.1368.

3-amino-1,8-bis(pent-4-en-1-yloxy) anthracene-9,10-dione (4b): Isolated yield: 77%, ¹H-NMR (Chloroform-d) δ 7.79 (d, J=7.6 Hz, 1H), 7.55 (t, J=8.0 Hz, 1H), 7.35-7.24 (m, 1H), 7.07 (d, J=2.2 Hz, 1H), 6.47 (d, J=2.3 Hz, 1H), 5.85-5.94 (m, 2H), 5.09 (dt, J=17.2, 1.8 Hz, 2H), 5.00 (dd, J=10.1, 1.9 Hz, 2H), 4.14 (q, J=6.2 Hz, 2H), 4.12-3.98 (m, 2H), 2.43-2.32 (m, 4H), 2.09-1.96 (m, 4H). ¹³C-NMR (Chloroform-d) δ 184.7, 180.8, 161.3, 158.9, 151.3, 138.0, 137.9, 136.3, 134.8, 132.8, 124.8, 120.1, 118.9, 116.0, 115.1, 104.6, 104.0, 77.3, 77.0, 76.7, 69.1, 68.7, 30.0, 30.0, 28.3, 28.2. HRMS (ESI) m/z: Calculated for C₂₄H₂₅NO₅Na[M+Na]⁺414.1700; Found 414.1681.

3-amino-1,8-bis(hex-5-en-1-yloxy) anthracene-9,10-dione (4c): Isolated yield: 64%, ¹H-NMR (Chloroform-d) δ 7.80 (d, J=7.6 Hz, 1H), 7.55 (t, J=8.0 Hz, 1H), 7.28 (s, 1H), 7.06 (d, J=2.2 Hz, 1H), 6.47 (d, J=2.1 Hz, 1H), 5.87 (ddt, J=16.9, 10.1, 6.6 Hz, 2H), 5.07 (d, J=17.1 Hz, 2H), 4.99 (d, J=10.1 Hz, 2H), 4.31 (s, 2H), 4.13 (t, J=6.5 Hz, 2H), 4.06 (t, J=6.5 Hz, 2H), 2.18 (q, J=7.0 Hz, 4H), 2.02-1.87 (m, 4H), 1.75-1.65 (m, 4H). ¹³C-NMR (Chloroform-d) δ 184.9, 181, 161.5, 159, 151.4, 138.8, 136.4, 134.9, 132.9, 124.8, 120.1, 119, 116.1, 114.8, 104.6, 104.1, 69.8, 69.4, 33.5, 33.5, 28.7, 28.6, 25.3. HRMS (ESI) m/z: Calculated for C₂₆H₃₀NO₄[M+H]⁺420.22; Found 420.2177.

3-amino-8-hydroxy-1-(pent-4-en-1-yloxy) anthracene-9,10-dione (10): Isolated yield: 56%, ¹H-NMR (Chloroform-d) δ 13.55 (s, 1H), 7.70 (d, J=7.3 Hz, 1H), 7.53 (t, J=7.9 Hz, 1H), 7.25 (d, J=8.7 Hz, 1H), 7.16 (d, J=1.8 Hz, 1H), 6.42 (d, J=1.7 Hz, 1H), 5.89 (ddt, J=13.2, 10.0, 6.6 Hz, 1H), 5.06 (dd, J=28.4, 13.3 Hz, 2H), 4.11 (t, J=6.5 Hz, 2H), 2.38 (dd, J=14.1, 7.0 Hz, 2H), 2.09-1.99 (m, 2H). ¹³C-NMR (Chloroform-d) δ 187, 183.5, 163.2, 162.5, 153.1, 151.5, 137.7, 137.5, 134.8, 132.8, 124.8, 118.5, 117.3, 115.6, 112.4, 106.5, 102.5, 68.7, 30, 29.8, 28.2. HRMS (ESI) m/z: Calculated for C₁₉H₁₈NO₄ [M+H]⁺324.12; Found 324.1226.

### General Procedure for the Synthesis of Compounds 5 and BW-AQ-336

Compound 4 (for compound 5) or compound 11 (for BW-AQ-336) (0.094 mmol) was dissolved in dry dioxane along with Et₃N (0.141 mmol). The solution was cooled down to 0° C. Further, chloroacetyl chloride (0.141 mmol) was added dropwise and stirred till the completion of the reaction, TLC (Hexane:ethyl acetate=2:1). Then, the reaction mixture was diluted with DCM (20 mL), washed by water (3×10 mL), brine (3×10 mL) and dried over anhydrous Na₂SO₄. Further, the solvent was reduced completely in-vacuo and purified by silica gel column chromatography, yielding desired yellow solid product. N-(4,5-bis(but-3-en-1-yloxy)-9,10-dioxo-9,10-dihydroanthracen-2-yl)-2-chloroacetamide (5a): Isolated yield: 92%, ¹H-NMR (Chloroform-d) δ 8.52 (s, 1H), 8.15 (d, J=2.2 Hz, 1H), 7.85 (dd, J=7.7, 1.1 Hz, 1H), 7.62 (dd, J=8.3, 7.7 Hz, 1H), 7.57 (d, J=2.1 Hz, 1H), 7.32 (dd, J=8.4, 1.1 Hz, 1H), 6.01-6.11 (m, 2H), 5.32-5.21 (m, 2H), 5.12-5.19 (m, 2H), 4.28-4.14 (m, 6H), 2.77-2.66 (m, 4H). ¹³C-NMR (Chloroform-d) δ 183.6, 180.9, 164.4, 160.3, 158.8, 141.5, 135.4, 134.6, 134.2, 134.1, 133.5, 124.5, 121.0, 120.2, 119.2, 117.5, 117.4, 109.8, 109.0, 69.3, 69.3, 42.9, 33.6, 33.5. HRMS (ESI) m/z: Calculated for C₂₄H₂₂NClO₅Na[M+Na]⁺462.1079; Found 462.1084.

2-chloro-N-(9,10-dioxo-4,5-bis(pent-4-en-1-yloxy)-9,10-dihydroanthracen-2-yl)acetamide (5b): Isolated yield: 96%, ¹H-NMR (Chloroform-d) δ 8.52 (s, 1H), 8.13 (d, J=2.1 Hz, 1H), 7.84 (dd, J=7.8, 1.1 Hz, 1H), 7.61 (t, J=8.0 Hz, 1H), 7.57 (d, J=2.1 Hz, 1H), 7.31 (dd, J=8.4, 1.1 Hz, 1H), 5.92 (m, 2H), 5.14 (q, J=1.8 Hz, 1H), 5.15-5.06 (m, 1H), 5.02 (dq, J=10.2, 1.5 Hz, 2H), 4.25 (s, 2H), 4.18 (dt, J=12.5, 6.3 Hz, 4H), 2.40, J=8.0, 6.7, 1.5 Hz, 4H), 2.05 (dtd, J=15.1, 6.5, 3.3 Hz, 4H). ¹³C-NMR (Chloroform-d) δ 183.7, 180.9, 164.2, 160.4, 159.0, 141.4, 137.8, 137.8, 135.5, 134.6, 133.5, 124.5, 121.0, 119.9, 119.0, 115.2, 109.6, 108.8, 69.0, 68.9, 42.8, 30.0, 29.9, 28.3, 28.1. HRMS (ESI) m/z: Calculated for C₂₆H₂₆NClO₅Na[M+Na]⁺490.1421; Found 490.1397.

N-(4,5-bis(hex-5-en-1-yloxy)-9,10-dioxo-9,10-dihydroanthracen-2-yl)-2-chloroacetamide (5c): Isolated yield: 86%, ¹H-NMR (Chloroform-d) δ 8.68 (s, 1H), 8.11 (d, J=1.8 Hz, 1H), 7.79 (d, J=7.6 Hz, 1H), 7.60-7.53 (m, 2H), 7.27 (d, J=8.5 Hz, 1H), 5.84 (ddt, J=16.9, 10.2, 6.6 Hz, 2H), 5.04 (dd, J=17.1, 1.5 Hz, 2H), 4.96 (d, J=10.2 Hz, 2H), 4.23 (s, 2H), 4.13 (dd, J=13.9, 6.7 Hz, 4H), 2.15 (q, J=7.1 Hz, 4H), 1.97-1.86 (m, 4H), 1.72-1.61 (m, 4H). ¹³C-NMR (Chloroform-d) δ 183.9, 181.1, 164.5, 160.6, 159.1, 141.7, 138.7, 135.5, 134.7, 133.6, 124.4, 120.9, 119.9, 119, 114.9, 114.8, 109.7, 108.9, 69.7, 69.7, 43, 33.5, 33.4, 28.6, 28.5, 25.2, 25.2. HRMS (ESI) m/z: Calculated for C₂₈H₃₁ClNO₅[M+H]⁺496.19; Found 496.1891.

2-chloro-N-(5-hydroxy-9,10-dioxo-4-((4-oxopentyl)oxy)-9,10-dihydroanthracen-2-yl)acetamide (BW-AQ-336): Isolated yield: 77%, ¹H-NMR (Chloroform-d) § 13.17 (s, 1H), 8.58 (s, 1H), 8.20 (s, 1H), 7.77 (d, J=7.3 Hz, 1H), 7.62 (dd, J=15.3, 7.2 Hz, 2H), 7.30 (d, J=8.2 Hz, 1H), 4.25 (d, J=6.3 Hz, 4H), 2.89 (t, J=6.8 Hz, 2H), 2.22 (d, J=6.5 Hz, 5H). ¹³C-NMR (Chloroform-d) δ 208.5, 187.5, 182.4, 164.6, 162.6, 162, 143.3, 136.6, 135.7, 132.6, 125.1, 77, 119, 117.4, 117.1, 110.3, 109, 68.7, 42.9, 39.7, 30.3, 29.8, 23, 22.8. HRMS (ESI) m/z: Calculated for C₂₁H₁₉ClNO₆[M+H]⁺416.09; Found 416.0887.

### General Procedure for the Synthesis of Compounds 6, 11, BW-AQ-260, 295, and 345

Into a 20 ml vial, compound 4 (for compound 6) or 5 (for BW-AQ-260,-295, and -345) or 10 (for compound 11) (0.03 mmol) was added followed by 3 ml DMF/Water (1:1). Then PdCl₂ (0.002 mmol) and CuCl (0.06 mmol) was added quickly, and a balloon of oxygen was plugged into the reaction mixture. Then the reaction was stirred overnight at room temperature. After TLC showed consumption of the starting material, the reaction mixture was diluted with water and extracted with DCM (5 ml, ×3). The combined organic layer was washed with water (50 ml) and brine (50 ml) successively and dried over anhydrous sodium sulphate. Then the solvent was removed in-vacuo, and the crude product was purified by silica-gel column chromatography (DCM/MeOH, 200/1). The product was isolated as a yellow solid.

3-amino-1,8-bis ((4-oxopentyl)oxy) anthracene-9,10-dione (6): Isolated yield: 56%. ¹H-NMR (Methanol-d₄) δ 7.73 (d, J=6.9 Hz, 1H), 7.61 (t, J=8.0 Hz, 1H), 7.43 (d, J=8.4 Hz, 1H), 7.00 (d, J=2.1 Hz, 1H), 6.57 (d, J=2.1 Hz, 1H), 4.15 (t, J=6.1 Hz, 2H), 4.09 (t, J=6.1 Hz, 2H), 2.86 (t, J=7.1 Hz, 4H), 2.20 (s, 6H), 2.14-2.07 (m, 4H). ¹³C-NMR (Dichloromethane-d₂) δ 208.7, 208.6, 184.7, 181, 161.4, 158.9, 152.3, 136.6, 135.2, 133.2, 124.9, 120.1, 119, 115.6, 104.6, 103.9, 68.8, 68.3, 39.9, 39.8, 30.2, 23.7, 23.6. HRMS (ESI) m/z: Calculated for C₂₄H₂₆NO₆[M+H]⁺424.465; Found 424.1769.

3-amino-8-hydroxy-1-((4-oxopentyl)oxy) anthracene-9, 10-dione (11): Isolated yield: 58%, ¹H-NMR (Chloroform-d) δ 13.56 (s, 1H), 7.69 (d, J=7.0 Hz, 1H), 7.53 (t, J=7.9 Hz, 1H), 7.24 (d, J=8.3 Hz, 1H), 7.15 (d, J=2.1 Hz, 1H), 6.44 (d, J=2.0 Hz, 1H), 4.13 (t, J=6.0 Hz, 2H), 2.88 (t, J=6.7 Hz, 2H), 2.21 (s, 3H), 2.19-2.12 (m, 2H). ¹³C-NMR (Chloroform-d) δ 208.9, 187, 183.4, 163, 162.5, 153.3, 137.5, 134.8, 132.8, 124.8, 118.5, 117.3, 112.3, 106.5, 102.4, 68.1, 39.5, 30.3, 23. HRMS (ESI) m/z: Calculated for C₁₉H₁₈NO₅[M+H]⁺340.12; Found 340.1175.

2-chloro-N-(9,10-dioxo-4,5-bis(3-oxobutoxy)-9,10-dihydroanthracen-2-yl)acetamide (BW-AQ-260): Isolated yield: 90%, ¹H-NMR (Chloroform-d) § 8.54 (s, 1H), 8.11 (d, J=2.1 Hz, 1H), 7.86 (dd, J=7.7, 1.1 Hz, 1H), 7.71-7.59 (m, 2H), 7.38-7.31 (m, 1H), 4.38-4.46 (m, 4H), 4.25 (s, 2H), 3.07 (td, J=6.2, 2.4 Hz, 4H), 2.39 (d, J=3.5 Hz, 6H). ¹³C-NMR (Chloroform-d) δ 206.8, 206.6, 183.3, 164.3, 159.9, 158.5, 141.6, 137.4, 135.5, 135.4, 134.5, 133.7, 129.0, 120.3, 119.7, 109.9, 109.4, 65.3, 65.2, 42.8, 42.7, 42.5, 31.0. HRMS (ESI) m/z: Calculated for C₂₄H₂₂NClO₇Na[M+Na]⁺494.0988; Found 494.0982.

2-chloro-N-(9,10-dioxo-4,5-bis ((4-oxopentyl)oxy)-9,10-dihydroanthracen-2-yl)acetamide (BW-AQ-295): Isolated yield: 87%, ¹H-NMR (Chloroform-d) δ 8.53 (s, 1H), 8.11 (d, J=2.1 Hz, 1H), 7.85 (dd, J=7.7, 1.1 Hz, 1H), 7.67-7.57 (m, 2H), 7.31 (dd, J=8.4, 1.1 Hz, 1H), 4.27-4.15 (m, 6H), 2.91 (td, J=6.9, 3.3 Hz, 4H), 2.30-2.11 (m, 10H). ¹³C-NMR (Chloroform-d) δ 208.6, 208.4, 183.5, 181.0, 164.3, 160.2, 158.7, 141.5, 135.5, 134.6, 133.7, 124.2, 120.7, 119.7, 119.2, 109.5, 109.0, 77.3, 77.0, 76.7, 68.4, 68.2, 42.8, 39.7, 39.5, 30.1, 30.1, 23.2, 23.1. HRMS (ESI) m/z: Calculated for C₂₆H₂₆NClO₇Na[M+Na]⁺522.1287; Found 522.1295.

2-chloro-N-(9,10-dioxo-4,5-bis ((5-oxohexyl)oxy)-9,10-dihydroanthracen-2-yl)acetamide (BW-AQ-345): Isolated yield: 39%, ¹H-NMR (Chloroform-d) δ 8.68 (s, 1H), 8.08 (d, J=1.9 Hz, 1H), 7.79 (d, J=7.5 Hz, 1H), 7.61-7.53 (m, 2H), 7.29-7.24 (m, 1H), 4.22 (s, 2H), 4.13 (q, J=6.0 Hz, 4H), 2.60 (t, J=6.7 Hz, 4H), 2.18 (s, 6H), 1.88 (tt, J=6.3, 4.0 Hz, 8H). ¹³C-NMR (Chloroform-d) δ 209.3, 209.2, 183.6, 181.1, 164.8, 160.3, 158.9, 142, 135.4, 134.6, 133.6, 124.1, 120.5, 119.8, 119.1, 109.6, 109.1, 69.4, 69.3, 43.2, 43.1, 29.9, 29.9, 28.5, 28.3, 20.4. HRMS (ESI) m/z: Calculated for C₂₈H₃₁ClNO₇[M+H]+528.18; Found 528.1793.

### Procedure for the Synthesis of BW-AQ-350

Compound 6 (0.025 mmol), propiolic acid (0.031), and DMAP (0.038) were mixed in dry DCM and stirred at an ice-bath temperature under argon protection. After 5 min, EDC (0.038) was added to the above reaction mixture. The reaction was monitored by TLC (DCM:MeOH=20:1). After completion of the reaction, DCM (10 mL) was added to the reaction solution and washed by water and brine. Next, the organic layer was dried over dry Na₂SO₄ and concentrated in-vacuo. The crude product was purified by silica gel column chromatography to yield a yellow product.

N-(9,10-dioxo-4,5-bis ((4-oxopentyl)oxy)-9,10-dihydroanthracen-2-yl) propiolamide (BW-AQ-350): Isolated yield: 82%, ¹H-NMR (Chloroform-d) δ 8.28 (s, 1H), 8.05 (s, 1H), 7.79 (d, J=7.6 Hz, 1H), 7.57 (dd, J=17.1, 9.2 Hz, 2H), 7.28 (d, J=8.6 Hz, 1H), 4.17 (t, J=5.9 Hz, 4H), 3.03 (s, 1H), 2.88 (dd, J=11.2, 6.8 Hz, 4H), 2.23 (s, 6H), 2.17 (td, J=6.3, 3.2 Hz, 4H). ¹³C-NMR (Chloroform-d) δ 208.9, 208.9, 208.8, 208.5, 183.6, 181.5, 181.1, 179, 160.3, 160, 158.8, 156.2, 149.9, 149.5, 142, 136.7, 135.5, 134.7, 134.6, 133.9, 133.8, 131.8, 129.2, 124.2, 122.3, 120.6, 120, 119.8, 119.5, 119.3, 118.6, 110, 109.7, 108.9, 103.5, 100.1, 75.4, 68.5, 68.3, 39.9, 39.7, 30.3, 30.2, 29.8, 23.3, 23.2. HRMS (ESI) m/z: Calculated for C₂₇H₂₅NO₇[M+Na]⁺498.1535; Found 498.1543.

### Procedure for the Synthesis of BW-AQ-349 and BW-AQ-353

Compound 6 (0.094 mmol) was dissolved in dry dioxane along with Et₃N (0.141 mmol). The solution was cooled down to 0° C. Acetyl chloride (for BW-AQ-349)/acryloyl chloride (for BW-AQ-353) (0.141 mmol) was added dropwise and stirred till the completion of the reaction, TLC (Hexane:ethyl acetate=2:1). After completion of the reaction, the reaction was diluted with water (10 mL) and extracted by DCM (3×10 mL). The combined organic phase was washed by brine (3×10 mL) and dried over anhydrous Na₂SO₄. Next, the solvent was completely removed in-vacuo and purified by silica gel column chromatography, yielding desired yellow solid product.

N-(9,10-dioxo-4,5-bis ((4-oxopentyl)oxy)-9,10-dihydroanthracen-2-yl)acetamide (BW-AQ-349): Isolated yield: 73%, ¹H-NMR (Methanol-d₄) δ 7.84 (dd, J=14.1, 4.9 Hz, 1H), 7.73 (dt, J=17.8, 5.9 Hz, 2H), 7.61 (dd, J=16.3, 8.2 Hz, 1H), 7.39 (dd, J=17.5, 8.5 Hz, 1H), 4.18-4.07 (m, 4H), 2.85 (td, J=7.0, 3.2 Hz, 4H), 2.21 (d, J=1.2 Hz, 6H), 2.12 (dt, J=11.5, 4.4 Hz, 7H). ¹³C-NMR (Chloroform-d) δ 209, 209, 183.9, 181.1, 169.2, 160.3, 158.8, 143.3, 135.2, 134.7, 133.6, 124.2, 119.7, 119.1, 109.5, 108.7, 77.4, 77.3, 77.1, 76.8, 68.4, 68.3, 40, 39.7, 30.3, 30.2, 24.9, 23.3. HRMS (ESI) m/z: Calculated for C₂₆H₂₇NO₇[M+H]⁺464.1788; Found 464.1036.

N-(9,10-dioxo-4,5-bis ((4-oxopentyl)oxy)-9,10-dihydroanthracen-2-yl) acrylamide (BW-AQ-353): Isolated yield: 44%, ¹H-NMR (Chloroform-d) § 8.61 (s, 1H), 8.28 (s, 1H), 7.71 (d, J=7.5 Hz, 1H), 7.53 (t, J=8.0 Hz, 1H), 7.45 (d, J=1.4 Hz, 1H), 7.23 (d, J=8.3 Hz, 1H), 6.39 (dt, J=16.8, 13.2 Hz, 2H), 5.80 (d, J=10.3 Hz, 1H), 4.15 (dd, J=11.1, 5.6 Hz, 4H), 2.87 (dd, J=12.6, 6.6 Hz, 4H), 2.24 (s, 6H), 2.16 (dd, J=11.3, 5.8 Hz, 4H). ¹³C-NMR (Chloroform-d) δ 209.1, 209, 183.8, 181.1, 164.1, 160.3, 158.8, 143.1, 135.2, 134.6, 133.7, 130.8, 129.2, 124.2, 119.8, 119.7, 119.1, 109.6, 108.9, 68.4, 68.3, 40, 39.8, 30.3, 30.2, 29.8, 23.3. HRMS (ESI) m/z: Calculated for C₂₇H₂₇NO₇[M+H]+=478.5130; Found 478.188.

### Procedure for the Synthesis of BW-AQ-354

Compound 6 (0.118 mmol) and Et₃N (0.354 mmol) were stirred in dry DCM at room temperature. Next, 2-chloroethanesulfonyl chloride (0.014 mmol) was added dropwise to the reaction mixture and stirred till the completion of the reaction. Further, the reaction mixture was diluted with water (10 mL), extracted with DCM (3×10 mL), dried over dry sodium sulphate, and concentrated in-vacuo. After purification by silica gel column chromatography, the intermediate compound (0.103 mmol) was further treated with TBAF (0.113 mmol) in THF (3 mL) and monitored by TLC till completion of the reaction. Next, the reaction was quenched by adding water (10 mL), extracted with DCM (3×10 mL), and the combined organic phase was further dried over dry sodium sulphate. After complete removal of the solvent in-vacuo, the crude product was purified by silica-gel column chromatography to yield desired yellow-solid product.

N-(9,10-dioxo-4,5-bis ((4-oxopentyl)oxy)-9,10-dihydroanthracen-2-yl) ethenesulfonamide (BW-AQ-354): Isolated yield: 77%, ¹H-NMR (Chloroform-d) δ 7.83 (d, J=7.2 Hz, 1H), 7.60 (t, J=8.0 Hz, 1H), 7.48 (d, J=1.9 Hz, 1H), 7.38 (s, 1H), 7.29 (d, J=7.9 Hz, 1H), 7.22 (d, J=2.1 Hz, 1H), 6.62 (dd, J=16.5, 9.8 Hz, 1H), 6.44 (s, 1H), 6.40 (s, 1H), 6.06 (d, J=9.8 Hz, 1H), 4.15 (dt, J=8.7, 6.0 Hz, 4H), 2.88 (t, J=6.7 Hz, 4H), 2.25-2.12 (m, 10H). ¹³C-NMR (Chloroform-d) δ 208.8, 208.7, 183.8, 181.1, 160.4, 158.8, 141.7, 136, 135.1, 134.5, 133.9, 129.6, 124.1, 120.6, 119.9, 119.4, 108.8, 108.5, 68.5, 68.4, 39.7, 39.6, 30.2, 29.8, 23.3, 23.1. HRMS (ESI) m/z: Calculated for C₂₆H₂₇NO: S [M+Na]⁺536.5610; Found 536.134.

### Procedure for the Synthesis of BW-AQ-365

A solution of BW-AQ-295 (0.068 mmol) and NaN₃ (0.136 mmol) was stirred at room temperature (Scheme 3). After overnight stirring, the solvent was removed in-vacuo and the compound was resuspended in DCM, washed by water, and dried over anhydrous Na₂SO₄. After concentrating under reduced pressure, the crude compound was purified using column chromatography to yield a yellow-solid product. 2-azido-N-(9,10-dioxo-4,5-bis ((4-oxopentyl)oxy)-9,10-dihydroanthracen-2-yl)acetamide (BW-AQ-365): Isolated yield: 99%, ¹H-NMR (CDCl₃) δ 8.55 (d, J=55.6 Hz, 1H), 8.07 (s, 1H), 7.79 (dd, J=7.6, 3.8 Hz, 1H), 7.64-7.48 (m, 2H), 7.29 (s, 1H), 4.18 (t, J=12.1 Hz, 6H), 2.88 (td, J=6.8, 3.2 Hz, 4H), 2.28-2.10 (m, 10H). ¹³C-NMR (CDCl₃) δ 209.01, 208.99, 208.97, 208.93, 183.55, 181.13, 165.76, 164.74, 160.17, 158.77, 142.10, 141.99, 135.37, 135.33, 134.60, 133.77, 133.73, 124.06, 120.44, 120.24, 119.70, 119.19, 119.16, 109.52, 109.13, 109.06, 77.48, 68.43, 68.30, 52.84, 43.09, 39.92, 39.88, 39.72, 30.26, 30.21, 23.24, 23.20. HRMS (ESI) m/z: Calculated for C₂₆H₂₇N₄O₇ [M+H]⁺507.1880; Found 507.1901.

### Results

Because the 1,8-hydroxyl or 1,8-alkoxyl substituted anthraquinone core is related to MDM2 downregulation, a class of compounds were developed by introducing an aliphatic ketone group at the R¹/R¹′-positions, while keeping the anthraquinone core intact. This development was based on two factors: an understanding of the sidechain's ability to tolerate modification and a plan to introduce a polar hydrogen bond acceptor group. Of note, an aliphatic ketone group is present in several FDA-approved drugs such as nabumetone, methadone and warfarin.

### Chemistry

The synthesis of the designed compounds (see Table 1) employed a similar approach as that of the previously published analogs. Specifically, the synthesis started with either rhein or its methyl ester (see Scheme 1). Alkylation with the corresponding alkenyl halide at the phenol hydroxyl groups of the anthraquinone core allowed for the introduction of the alkenyl group as a latent ketone moiety. Then the ester group of intermediates 1 was hydrolyzed before introducing an azido group on the free carboxylic acid of 2, setting up a Curtis rearrangement for the subsequent formation of the arylamino group on 4. The introduction of various acyl or sulfonyl groups led to the installation of different R² in the target compounds. The conversion of the alkene moiety to a ketone group was conducted as the last step through Wacker oxidation for the synthesis of compounds with the chloroacetyl moiety (BW-AQ-260,-295,-345). However, for analogs with functional groups that are sensitive to Wacker oxidation, including BW-AQ-350,-353, and -354, the oxidative conversion of the alkenyl moiety to a ketone group was conducted as the penultimate step.

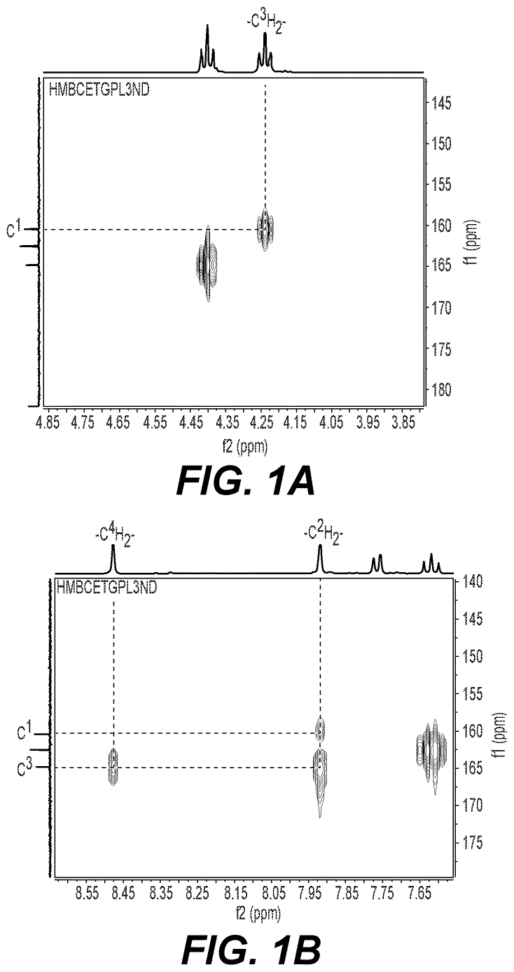

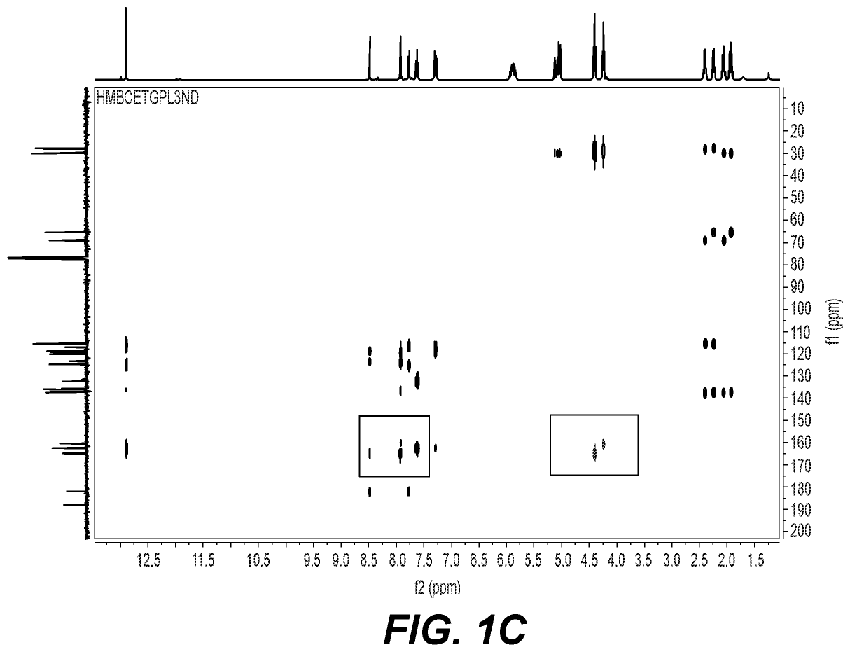

In addition to the dialkylated analogs, a mono-substituted anthraquinone analog (BW-AQ-336) was synthesized by carefully controlling the alkylation conditions (Scheme 2). This compound allows for probing the effect of mono-substitution vs. di-substitution of the hydroxyl groups present on the anthraquinone core. Using HMBC 2D-NMR (FIGS. 1A-1C), it was found that the methylene proton of the alkenyl side-chain correlated with the aromatic carbon at position 2, which can be validated by the net correlation shown in FIGS. 1A-1B. Thus, the alkenyl substitution position was confirmed to be at the R¹′ position. The preferential formation of this regioisomer could be due to the presence of an electron-withdrawing carboxyl group on the right phenyl ring of the anthraquinone moiety, which makes the hydroxyl group at the R¹′ position (general structure in Table 1) slightly more acidic than the one on the left side and thus easier to deprotonate.

### In-vitro Structure-activity Relationship (SAR) Studies

Modification at the R¹/R¹′ Position

In designing the anthraquinone analogs, SAR information was considered. Specifically, the anthraquinone core was needed for activity and the R¹/R¹′ positions were found to tolerate some degree of variations. Additionally, an ethyl or a hydroxyethyl (BW-AQ-238) (Table 1, Entry 1) substitution at the R¹/R¹′ position allowed for retention of their ability to lead to MDM2 degradation and p53 activation. At the R² position, a chloroacetyl group was found to be involved in activity in downregulating MDM2 in the ALL cells. Bearing these SAR findings in mind, the initial attempt was focused on modifying the side chain (R¹/R¹′) positions while keeping R² as the chloroacetyl group (Table 1, Entries 2-5). Incorporating a ketone group as a hydrogen-bond acceptor was of particular interest. To explore the SAR of the chain length, C₄ to C₆ in chain length were studies. Table 1 shows the analogs designed.

TABLE 1
Analogs design

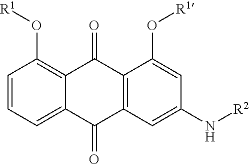

| | | | | IC₅₀ (μM) |
|---|---|---|---|---|
| Entry | BW-AQ-# | R¹/R^{1′} | R² | in EU-1 cells |
| 1 | 238 | R¹ =R^{1′} = 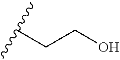 [SMILES: *CCO] | 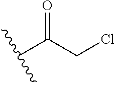 [SMILES: *C(=O)CCl] | 0.74 ± 0.12 |
| 2 | 260 | R¹ = R^{1′} = 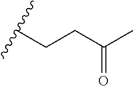 [SMILES: *CCC(C)=O] | 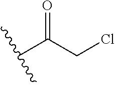 [SMILES: *C(=O)CCl] | 0.45 ± 0.02 |
| 3 | 295 | R¹ = R^{1′} = 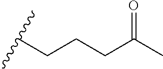 [SMILES: *CCCC(C)=O] | 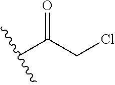 [SMILES: *C(=O)CCl] | 0.43 ± 0.06 |
| 4 | 345 | R¹ = R^{1′} = 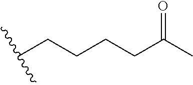 [SMILES: *CCCCC(C)=O] | 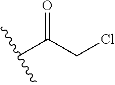 [SMILES: *C(=O)CCl] | 0.79 ± 0.03 |
| 5 | 336 | R¹ = H, R^{1′} = 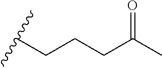 [SMILES: *CCCC(C)=O] | 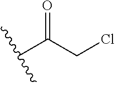 [SMILES: *C(=O)CCl] | 0.84 ± 0.02 |
| 6 | 350 | R¹ = R^{1′} = 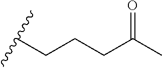 [SMILES: *CCCC(C)=O] | 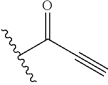 [SMILES: *C(=O)C#C] | 0.19 ± 0.01 |
| 7 | 353 | R¹ = R^{1′} = 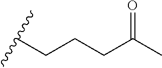 [SMILES: *CCCC(C)=O] | 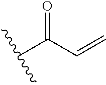 [SMILES: *C(=O)C=C] | 6.21 ± 0.45 |
| 8 | 354 | R¹ = R^{1′} = 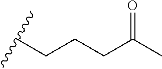 [SMILES: *CCCC(C)=O] | 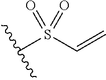 [SMILES: *S(=O)(=O)C=C] | 3.45 ± 0.45 |
| 9 | 349 | R¹ = R^{1′} = 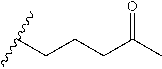 [SMILES: *CCCC(C)=O] | 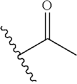 [SMILES: *C(C)=O] | >100 |
| Control compounds | | | | |
| 10 | Propiolamide | (CHCCONH₂) | | 33.5 ± 7.8 |
| 11 | Doxorubicin | | | 0.15 ± 0.02 |

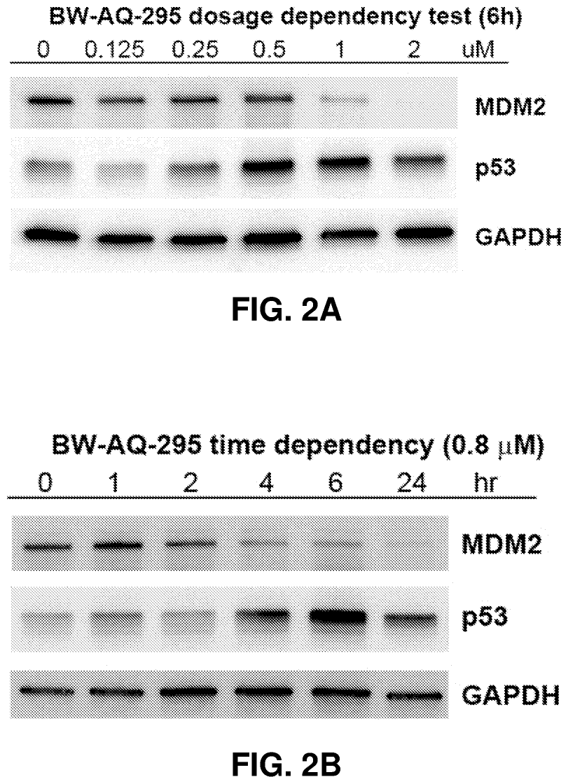

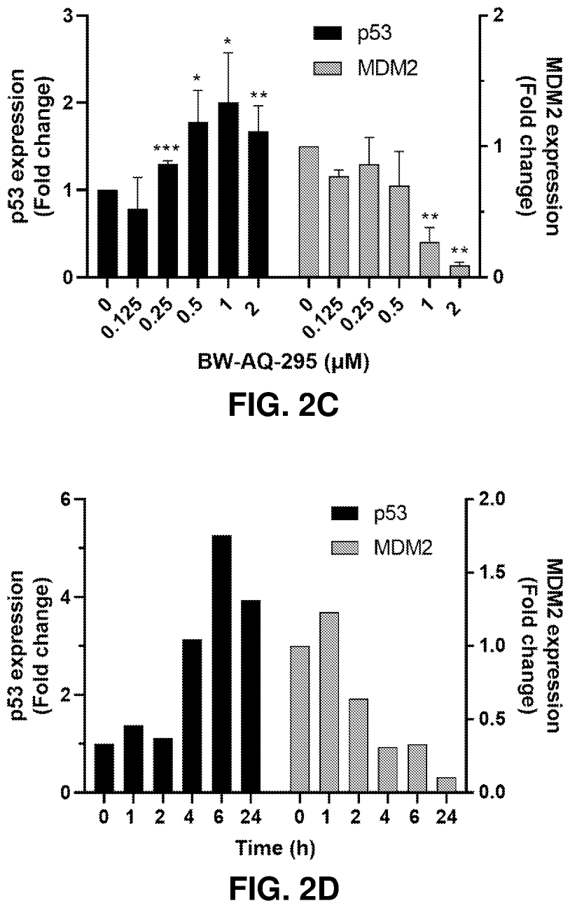

With the installation of alkyl ketone groups at the R¹ and R¹′ positions, the cytotoxicity of these analogs were evaluated in EU-1 cells using BW-AQ-238 as the positive control. The C₄ and C₅ ketone derivatives BW-AQ-260 and BW-AQ-295 were about 3-fold more potent than BW-AQ-238 (see Table 1). Further increasing the chain length to C₆ decreased the potency by about 1-fold compared to BW-AQ-295. Further, treatment of EU-1 cells with BW-AQ-336 (mono pentan-2-one substitution) resulted in a significant decrease in potency (about 2-fold). Such results show that the substitution on both phenol hydroxyl groups is of benefit for preserving the potency. With BW-AQ-295 being the most potent, experiments were conducted to confirm its degradation of MDM2 using EU-1 cells. Western-blot studies were performed accordingly, and the results show the ability of BW-AQ-295 to decrease MDM2 level and increase p53 level in a dose- and time-dependent manner (FIGS. 2A-2D). At 2 μM concentration, BW-AQ-295 decreased MDM2 to an almost undetectable level at 6 h. At 0.8 μM, decreased MDM2 and increased p53 level were observed from the 4-h time point after treatment.

### Modification at the R² Position

Having BW-AQ-295 with the introduction of terminal alkyl ketone groups at the R¹ and R¹′ positions, the chloroacetamide group of BW-AQ-295 (R² substitution) was further studied to determine whether it can be replaced by other electrophiles, such as acrylamide, propynamide, and ethensulfonamide (Table 1). Compared to BW-AQ-295, the acrylamide analog BW-AQ-353 was about 15-fold less potent. The sulfonamide analog BW-AQ-354 was found to be more potent than BW-AQ-353. The propynamide analog BW-AQ-350 showed a 2-fold increase in potency compared to BW-AQ-295, to a level that is comparable to that of doxorubicin (Table 1, Entry 11). The lack of activity in the acetamide analog BW-AQ-349 (Table 1, Entry 9) and the propiolamide analog (Table 1, Entry 10) show a role for the electrophilic moiety and the anthraquinone core. At least, such results demonstrate the need to explore the effect of their chemical reactivity as an electrophile on their cytotoxicity.

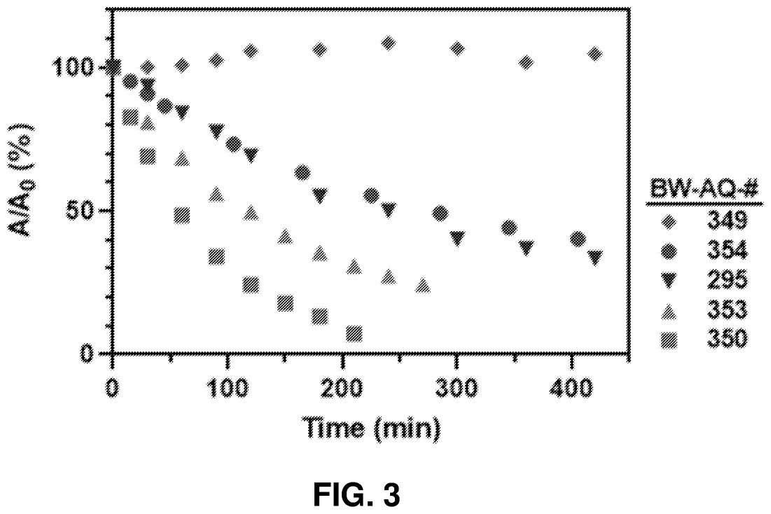

Under physiological conditions, the electrophilic moiety of these anthraquinone analogs is capable of reacting with intracellular nucleophiles such as a protein thiol group to various degrees. Therefore, the general reactivity of the developed anthraquinone-analogs (Table 1, Entries 3, 6-9) towards a commonly used model thiol, N-acetyl cysteine (NAC), were investigated using HPLC. A comparative analysis of the pseudo-first-order kinetics of the anthraquinone-analogs with NAC at 37° C. in phosphate-buffered saline (PBS) solution provided the order of reactivity being BW-AQ-350 >BW-AQ-353 >BW-AQ-295 ~ BW-AQ-354 >>BW-AQ-349 (propynamide>acrylamide>chloroacetamide≈sulfonamide>>acetamide) (FIG. 3). BW-AQ-350 showed the highest reactivity towards NAC among the tested analogs. BW-AQ-349 remained unchanged during the study duration (>400 min) because of its lack of an electrophilic group. These results agree with the understanding that a faster thiol reaction with the Michael acceptor with an alkynyl moiety than that with an alkenyl electrophile. Interestingly, the reactivity of the electrophilic moiety is not in the same order as the observed cytotoxicity. For example, BW-AQ-353 was found to be more reactive to NAC than BW-AQ-295, and yet showed much lower potency. Therefore, the chemical reactivity of the electrophile does not seem to be the determining factor for potency. This is understandable since there is no information indicating that an electrophile is an absolute requirement for activity. Further, even if a reaction involving an electrophile is involved, there are other factors that help shape both potency and selectivity. One will need much more work including extensive structural studies to truly assess the contribution of each factor.

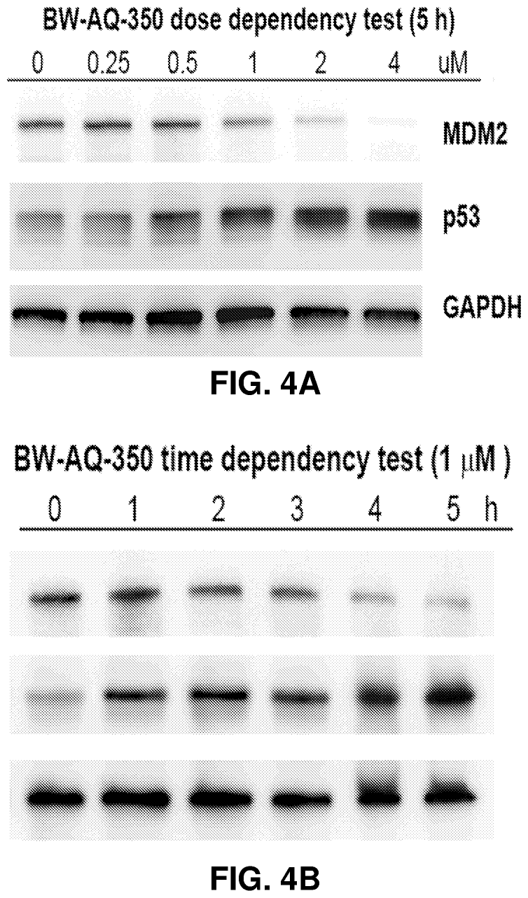

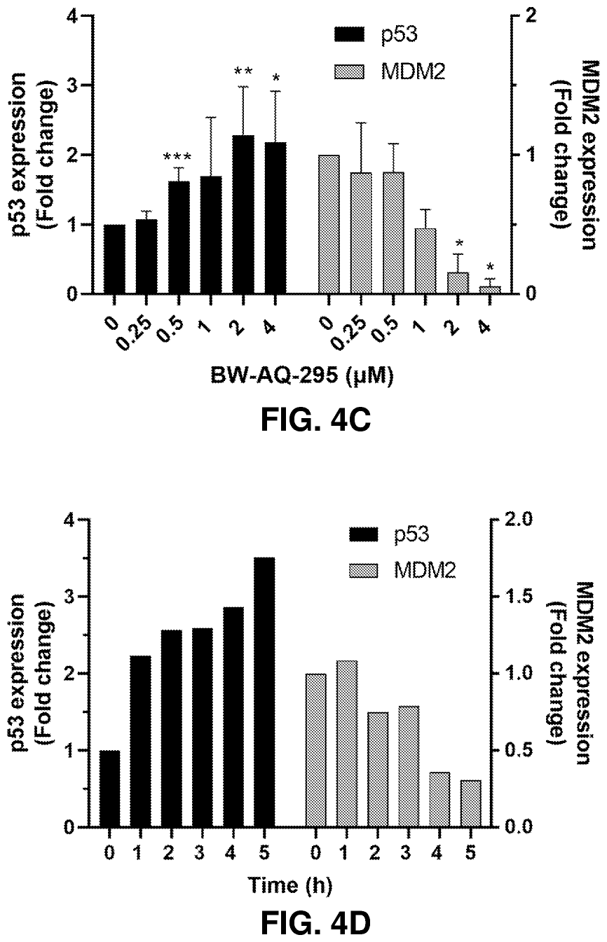

With its high potency, the propynamide analog BW-AQ-350 was selected as a representative to test its effect on downregulating MDM2 by Western-blot studies in EU-1 cells. It was found that BW-AQ-350 induced decrease in MDM2 levels in a dose- and time-dependent fashion (FIGS. 4A-4D). To this end, BW-AQ-350 was found to be the most potent candidate in inhibiting the proliferation of EU-1 cells by downregulating MDM2, thus upregulating the p53 level. The cytotoxicity of BW-AQ-295 and BW-AQ-350 in other cell lines was tested to study their general activity. Table 2 shows the cytotoxicity of BW-AQ-295 and BW-AQ-350 in cell lines harboring WT-p53, including MCF7, RS4;11, as well as HeLa cells with aberrant-p53. A non-cancerous embryonic rat cardiomyoblasts cell line H₉c2 and human embryonic kidney cell line HEK-293 were also included. These two compounds retained their high potency against RS4;11 ALL cells which further support their cytotoxicity against leukemia cells harboring WT-p53 (Table 2, Entry 1). Further, in the cancerous epithelial cells, BW-AQ-295 showed over 2-fold less potency in HeLa cells (Table 2, Entry 3) when compared to MCF7 cells (Table 2, Entry 2), demonstrating the role of WT-p53 for the higher cytotoxicity of BW-AQ-295. In both HeLa and MCF7 cancer cell lines, BW-AQ-350 showed significant lower potency than BW-AQ-295. This trend can also be seen in HEK-293 cells, though to a less extent (Table 2, Entry 5). In embryonic H₉c2 cells, BW-AQ-350 showed much higher potency than BW-AQ-295 (Table 2, Entry 4), similar to the trend seen in EU-1 cells. The difference in the order of potency of these two compounds shown in different cell lines warrant further in-depth mechanistic studies. Detailed mechanistic studies need to use cell lines with carefully controlled genetic phenotypes.

Furthermore, the IC₅₀ in the micromolar range demonstrates aside from activation of p53 through MDM2 down-regulation, BW-AQ-295 and BW-AQ-350 may also induce cell death through other p53-independent pathways.

TABLE 2
Cytotoxicity in different cell lines

IC₅₀ (μM)

| Entry | Cell lines | BW-AQ-295 | BW-AQ-350 |
|---|---|---|---|
| 1 | RS4; 11 | 0.69 ± 0.10 | 0.52 ± 0.07 |
| 2 | MCF7 | 0.95 ± 0.17 | 4.93 ± 0.45 |
| 3 | HeLa | 2.31 ± 0.08 | 4.67 ± 0.25 |
| 4 | H9c2 | 1.12 ± 0.20 | ca. 0.30 ± 0.08 |
| 5 | HEK-293 | 1.16 ± 0.34 | 2.35 ± 0.69 |

### Anticancer Evaluation of BW-AQ-365

BW-AQ-350 showed high reactivity towards nucleophile such as thiol and amino acid. BW-AQ-295 also reacts with thiol species. Thus, efforts were made to attenuate reactivity while maintaining the anticancer activity through degrading MDM2 and induce cell apoptosis. Conversion of the reactive chloroacetyl group into a less reactive azidoacetyl group, while maintaining the high bioactivity empowered by the alkylketone sidechain afforts compound BW-AQ-365. BW-AQ-365 showed less reactivity towards thiol, and the anticancer activity of BW-AQ-365 is more potent than BW-AQ-295 in RS4;11 cells, demonstrating its high specificity and less toxicity concerns for anticancer drug development.

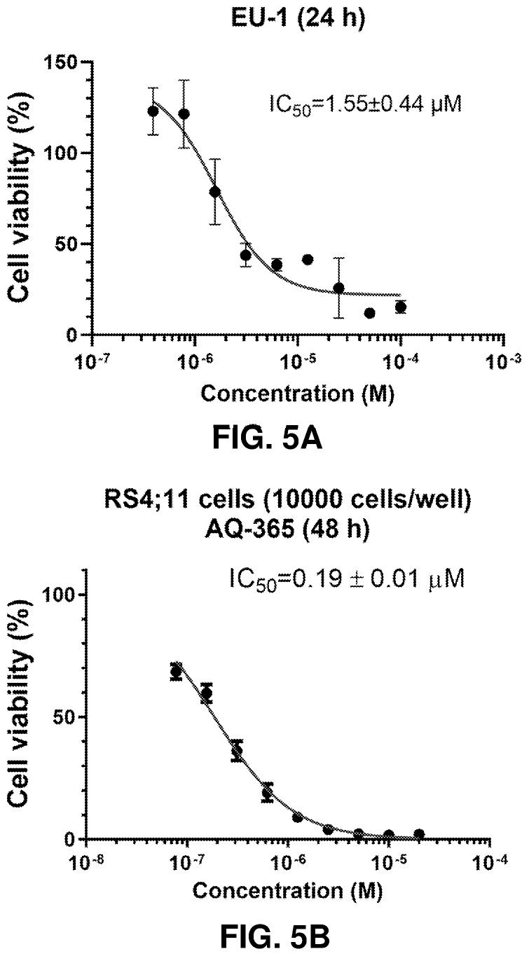

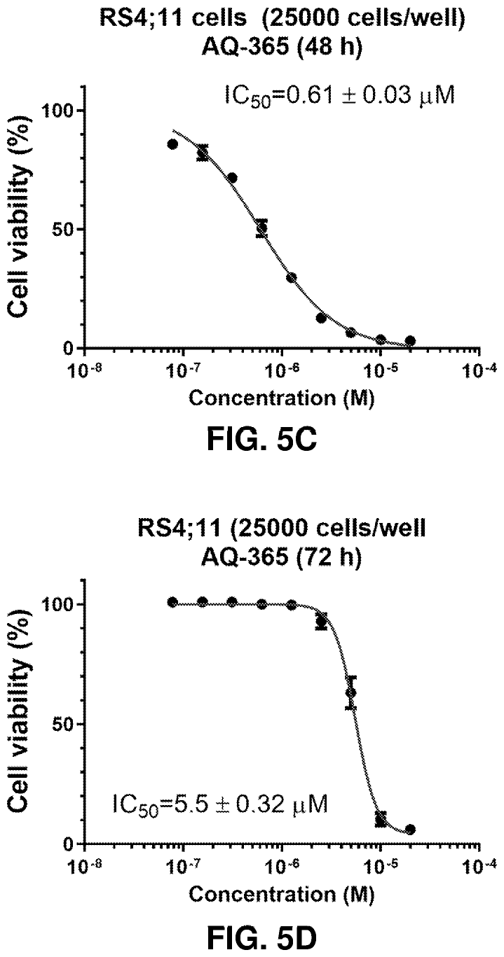

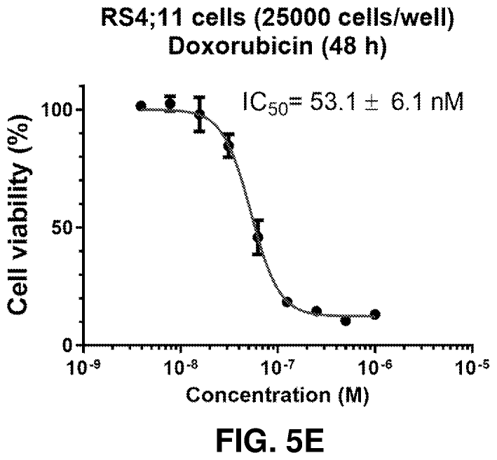

Further, in the in-vitro cytotoxicity study, the potency of BW-AQ-365 is inversely proportional to the cell confluency (Table 3 and FIGS. 5A-5E). It shows that BW-AQ-365 could still react and be compromised by the cellular environment. However, such effects are much less than BW-AQ-295 and BW-AQ-350.

TABLE 3
In-vitro cytotoxicity result of BW-AQ-365

| | Cell density | | | |
|---|---|---|---|---|
| Cell line | (cells/well) | Time (h) | IC₅₀ (±SD) | |
| EU-1 (in-house cell line) | 25000 | 24 | 1.55 ± 0.44 | μM |
| RS4; 11 | 10000 | 48 | 0.19 ± 0.01 | |
| RS4; 11 | 25000 | 48 | 0.61 ± 0.03 | μM |
| RS4; 11 | 25000 | 72 | 5.5 ± 0.32 | μM |
| RS4; 11 + Doxorubicin | 25000 | 48 | 53.1 ± 6.1 | nM |
| (Positive control) | | | | |

Preliminary study also showed that BW-AQ-365 could dose-dependently decrease MDM2 level in RS4;11 cells. Experiments to confirm dose along with the time-dependency will be assessed. The induction of cancer cell apoptosis will also be evaluated.

### CONCLUSION

Structural modifications of anthraquinone analgos to improve potency while maintaining its ability to downregulate MDM2 were conducted. At first, a hydroxyl group of the lead compound (BW-AQ-238) was modified to a hydrogen bond acceptor ketone group at the terminal position of the alkyl sidechain. With such modification, BW-AQ-260 exhibited significant improvement in potency (IC₅₀=0.45+0.02 μM) against EU-1 cells. By changing the alkyl chain length of BW-AQ-260, the C₅ ketone side-chain analog BW-AQ-295 was found to possess improved cytotoxicity, while further extending the sidechain length or removing one side chain was found to lead to inferior activity. Further, the R² substituent of the BW-AQ-295 was examined. Replacing the chloroacetamide group with a propynamide group resulted in the most potent compound, BW-AQ-350 (IC₅₀=0.19+0.01 μM), in this series of anthraquinone analogs. The activity was comparable to that of doxorubicin in the same ALL cell line. Further, Western-blot experiments showed the ability for BW-AQ-295 and BW-AQ-350 to downregulate MDM2 and upregulate p53 in EU-1 cells in a time- and dose-dependent fashion. Satisfying the key features of Lipinski's Rule of Five, BW-AQ-350 and BW-AQ-295 are potent candidates for treating ALL. Further, BW-AQ-365 showed less reactivity towards thiol, and the anticancer activity of BW-AQ-365 is more potent than BW-AQ-295 in RS4;11 cells, demonstrating its high specificity and less toxicity concerns for anticancer drug development.

### REFERENCES

Turcotte, L. M., et al., Blood 2021, 138 (Supplement 1), 663-663.
Tyner, J. W., et al., Leukemia 2012, 26 (4), 623-632.
Yuan, S., et al., Leukemia 2021.
Amaral, J. D., et al., Discov Med 2010, 9 (45), 145-152.
Wade, M., et al., Nat Rev Cancer 2013, 13 (2), 83-96.
Piette, J., et al., Oncogene 1997, 15 (9), 1001-1010.
Urso, L., et al., Crit Rev Oncol Hematol 2016, 97, 220-30.
Watanabe, T., et al., Leuk Lymphoma 1996, 21 (5-6), 391-7.
Kojima, K., et al., Exp Hematol 2016, 44 (9), 791-798.
Gu, L., et al., Leukemia 2008, 22 (4), 730-739.
Trino, S., et al., Front Pharmacol 2016, 7 (491).
Han, X., et al., Mod Pathol 2007, 20 (1), 54-62.
Anifowose, A., et al., Med Chem Res 2020, 29 (7), 1105-1121.
Shangary, S., et al., ANNU REV PHARMACOL TOXICOL 2009, 49, 223-41.
Zhu, N., et al., Mol Cancer Ther 2008, 7 (5), 1101-9.
Trino, S., et al., Oncotarget 2016, 7 (11), 12951-61.
Ravandi, F., et al., Leuk Res 2016, 48, 92-100.
Bernal, F., et al., J Am Chem Soc 2007, 129 (9), 2456-2457.
Bernal, F., et al., Cancer Cell 2010, 18 (5), 411-22.
Zhang, H., et al., PLoS One 2014, 9 (6), e100960.
Huang, M., et al., Mol Cancer Ther 2013, 12 (2), 184-94.
Li, Y., et al., J Med Chem 2019, 62 (2), 448-466.
He, S., et al., Acta Pharm Sin B 2021, 11 (6), 1617-1628.
Anifowose, A., et al., Bioorg Med Chem Lett 2020, 30 (2), 126786.
Draganov, A. B., et al., Bioorg Med Chem 2019, 27 (17), 3860-3865.
Anifowose, A., et al., Med Chem Res 2020, 29 (7), 1199-1210.
Yang, X., et al., ChemMedChem 2011, 6 (12), 2294-301.
Gu, L., et al., Mol Cancer Ther 2018, 17 (2), 497-507.
Malik, M. S., et al., RSC Adv 2021, 11 (57), 35806-35827.
Damiani, R. M., et al., Arch Toxicol 2016, 90 (9), 2063-2076.
Paul, F., et al., J Neurol Neurosurg Psychiatry 2007, 78 (2), 198-200.
Menna, P., et al., Chem Res Toxicol 2002, 15 (9), 1179-89.
Singal, P. K., et al., Mol Cell Biochem 2000, 207 (1-2), 77-86.
Liu, J., et al., Am J Physiol Heart Circ Physiol 2008, 295 (5), H₁₉₅₆-H₁₉₆₅.
Guin, P. S., et al., Int J Electrochem 2014, 2014, 517371.
Fei, J., et al., Bioelectrochemistry 2007, 70 (2), 369-374.
Pan, L., et al., Chem Commun 2018, 54 (22), 2759-2762.
Younis, I. R., et al., J Clin Pharmacol 2019, 59 (8), 1035-1043.
Gehringer, M., et al., J Med Chem 2019, 62 (12), 5673-5724.
Cully, M., Nat Rev Drug Discov 2020, 19 (11), 754.
Singh, J., et al., Nat Rev Drug Discov 2011, 10 (4), 307-317.
Sutanto, F., et al., RSC Med Chem 2020, 11 (8), 876-884.
Mons, E., et al., J Am Chem Soc 2019, 141 (8), 3507-3514.
Amishiro, N., et al., Bioorg Med Chem 2000, 8 (7), 1637-1643.
Dimmock, J. R., et al., J Med Chem 2001, 44 (4), 586-93.
Van Herck, N., et al., Angew Chem Int Ed Engl 2020, 59 (9), 3609-3617.
Henise, J. C., et al., J Med Chem 2011, 54 (12), 4133-46.
Harada, H., et al., Bioorg Med Chem 2001, 9 (11), 2955-2968.
Flanagan, M. E., et al., J Med Chem 2014, 57 (23), 10072-10079.
Petri, L., et al., Bioorg Med Chem 2020, 28 (7), 115357.
Flanagan, M. E., et al., J Med Chem 2014, 57 (23), 10072-9.
Costa, A. M., et al., J Org Chem 2021, 86 (10), 7107-7118.
Kobayashi, T., et al., J Am Chem Soc 2016, 138 (45), 14832-14835.
Li, Q., et al., Cell 2020, 182 (1), 85-97.e16.
Alkhalaf, M., et al., J Endocrinol 2003, 179 (1), 55-62.
Zhao, Y., et al., J Med Chem 2013, 56 (13), 5553-61.
Hoppe-Seyler, F., et al., J Virol 1993, 67 (6), 3111-7.
Xie, et al., Appl Microbiol Biotechnol 2018, 102 (14), 5965-5975.
Watkins, S. J., et al., In Vitro Cell Dev Biol Anim 2011, 47 (2), 125-131.
Lin, Y. -C., et al., Nat Commun 2014, 5 (1), 4767.
Belzile, J. -P., et al., Cancer Gene Ther 2003, 10 (8), 637-646.
Holbeck, S. L., et al., Mol Cancer Ther 2010, 9 (5), 1451-60.
Chen, Y., et al., Front Pharmacol 2018, 9, 1453.
Lipinski, C. A., et al., Adv Drug Deliv Rev 2001, 46 (1-3), 3-26.
Anifowose, A. “Design and Synthesis of Anticancer Agents by Targeting p53 Degradation and Photodynamic Therapy,” Chemistry Dissertaions, Dec. 16, 2019

1. A compound having the structure of 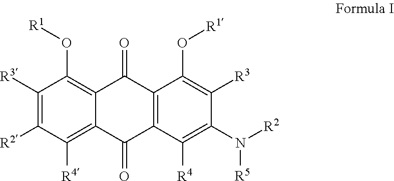
wherein
(a) R¹ and R¹′ are independently a hydrogen, a hydroxyalkyl, or carbonyl (e.g., a keto), wherein at least one of R¹ and R¹′ is not hydrogen;
(b) R² is an electrophilic group;
(c) R⁵ is a hydrogen, a substituted or unsubstituted alkyl, a substituted or unsubstituted alkenyl, or a substituted or unsubstituted alkynyl;
(d) R³, R⁴, R²′, R³′, and R⁴′ are independently a hydrogen, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclyl, a halogen, a hydroxyl, an amino, an amido, an azo, an alkoxy, a polyether, a thiol, a cyano, a nitro, or a carbonyl;
(e) each substituent is independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, an carboxyl, an amino, an amido, an azido, an oxo, a silyl, a sulfinyl, a sulfonyl, a sulfonic acid, a phosphonium, a phosphanyl, a phosphoryl, or a phosphonyl.

2. The compound of claim 1, wherein R² is a carbonyl, a sulfinyl, a sulfonyl, or a sulfamoyl.

3. The compound of claim 1, wherein R² is 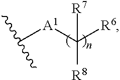
and wherein
A¹ is 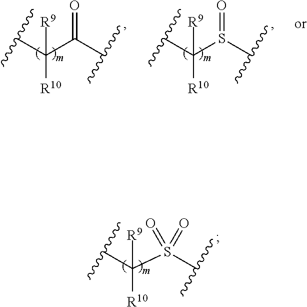
n and m are independently an integer from 0 to 6, from 0 to 5, from 0 to 4, from 0 to 3, or from 0 to 2, such as 1 or 0;
R⁶ is a halogen (e.g., fluorine, chlorine, bromine, or iodine), a cyano, an azido, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, or a substituted or unsubstituted heterocyclyl; and
R⁷—R¹⁰ are independently a hydrogen, a substituted or unsubstituted alkyl, a substituted or unsubstituted alkenyl, a substituted or unsubstituted alkynyl, a halogen, a hydroxyl, an amino, an amido, an alkoxy, an azo, a polyether, a thiol, a cyano, an azido, a nitro, a nitrile, a carbonyl, a sulfinyl, a sulfonyl, or a sulfamoyl.

4. The compound of claim 1, wherein R² is 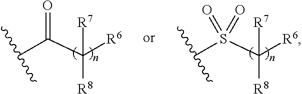
and wherein
n is an integer from 0 to 3, from 0 to 2, 0 or 1;
R⁶ is a halogen (e.g., fluorine, chlorine, bromine, or iodine), a cyano, an azido, a substituted or unsubstituted alkenyl, or a substituted or unsubstituted alkynyl; and
R⁷ and R⁸ are independently a hydrogen, a substituted or unsubstituted alkyl, a substituted or unsubstituted alkenyl, a substituted or unsubstituted alkynyl, a halogen, a cyano, or an azido.

5. The compound of claim 4, wherein R⁶ is a halogen (e.g., fluorine, chlorine, bromine, or iodine), an azido, an unsubstituted C₂-C₆ alkenyl, or an unsubstituted C₂-C₆ alkynyl, such as a chlorine, an unsubstituted ethylenyl, an unsubstituted propylenyl, an unsubstituted ethynyl, or an unsubstituted propynyl.

6. The compound of claim 1, wherein R¹ and R¹′ are independently a hydrogen, 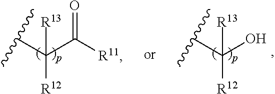
and wherein
p is an integer from 0 to 6, from 0 to 5, from 0 to 4, from 1 to 5, from 2 to 5, from 1 to 4, or from 2 to 4, such as 3; and
R¹¹—R¹³ are independently a hydrogen, a substituted or unsubstituted alkyl, a substituted or unsubstituted alkenyl, a substituted or unsubstituted alkynyl, a halogen, a hydroxyl, an amino, an amido, an alkoxy, an azo, an azido, a polyether, a thiol, a cyano, a nitro, or a nitrile.

7. The compound of claim 6, wherein R¹¹ is a hydrogen, a hydroxyl, or a substituted or unsubstituted alkyl; and R¹² and R¹³ are independently a hydrogen or a substituted or unsubstituted alkyl.

8. The compound of claim 6, wherein R¹¹ is an unsubstituted C₁-C₆ alkyl, an unsubstituted C₁-C₅ alkyl, an unsubstituted C₁-C₄ alkyl, an unsubstituted C₁-C₃ alkyl, an unsubstituted methyl or ethyl, such as methyl.

9. The compound of claim 6, wherein R¹² and R¹³ are hydrogen.

10. The compound of claim 1, wherein R⁵ is hydrogen.

11. The compound of claim 1, wherein R³, R⁴, R²′, R³′, and R⁴′ are independently a hydrogen or a substituted or unsubstituted alkyl.

12. The compound of claim 1, wherein R³, R⁴, R²′, R³′, and R⁴′ are hydrogen.

13. The compound of claim 1, wherein the compound has the structure of:

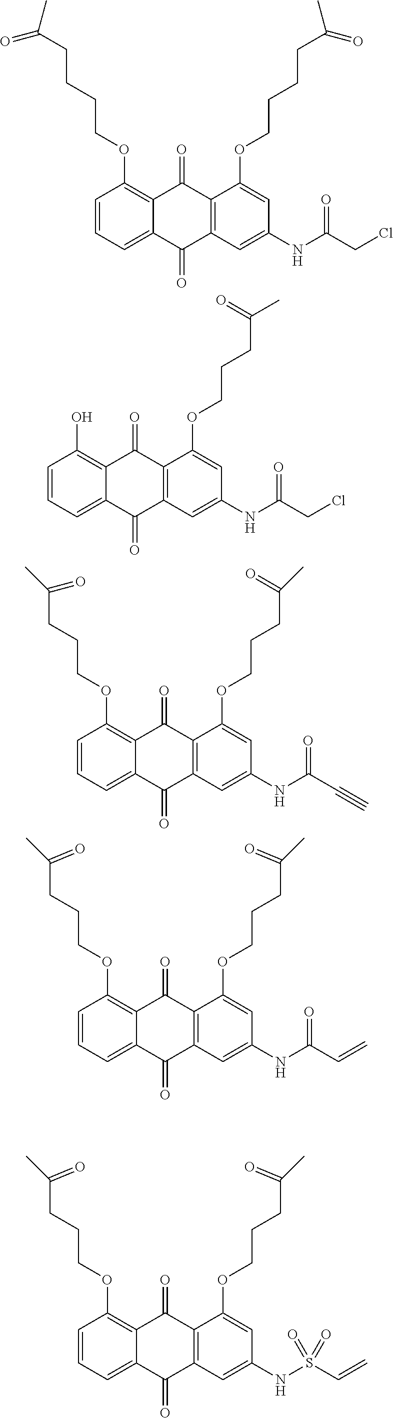

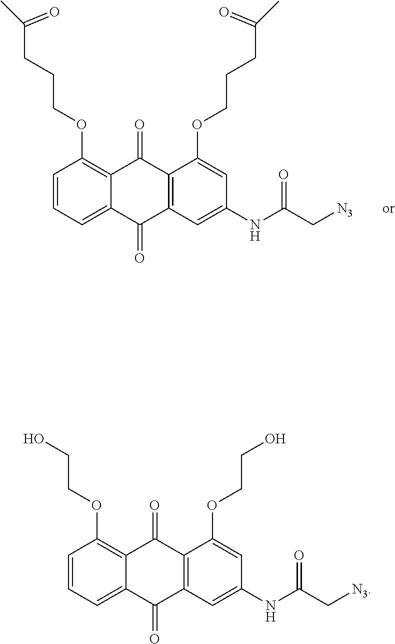 [SMILES: CC(=O)CCCOc1cccc2c1C(=O)c1c(OCCCC(C)=O)cc(NC(=O)CN=[N+]=[N-])cc1C2=O ; [N-]=[N+]=NCC(=O)Nc1cc(OCCO)c2c(c1)C(=O)c1cccc(OCCO)c1C2=O]

14. The compound of claim 1, wherein the compound is

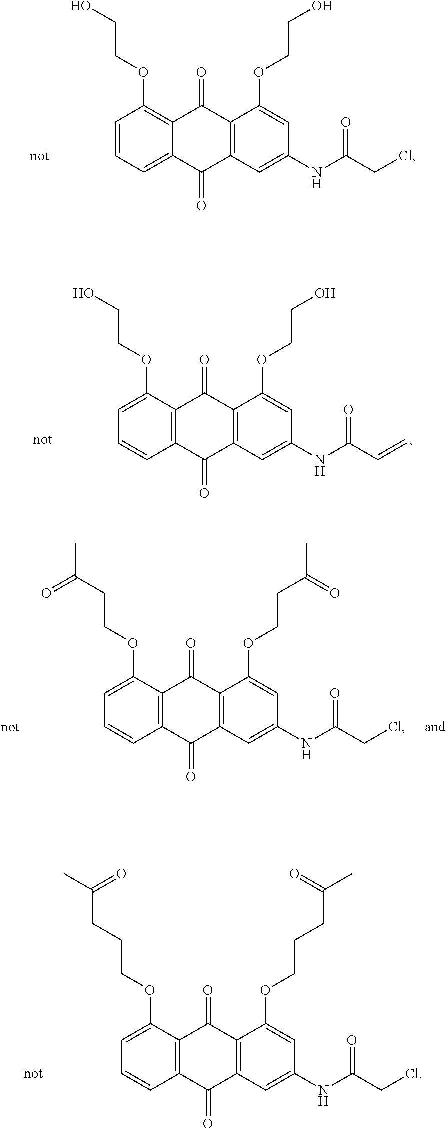

15. The compound of claim 1, wherein the compound has an IC₅₀ value against test cancer cells comparable or lower than an IC₅₀ value of BW-AQ-101 and/or BW-AQ-238 against the same test cancer cells, tested under the same conditions.

16. A pharmaceutical formulation comprising
one or more compounds of claim 1; and
a pharmaceutically acceptable carrier and/or excipient.

17. The pharmaceutical formulation of claim 16, wherein the one or more compounds are in an effective amount to treat or ameliorate one or more symptoms associated with a cancer in a subject.

18. The pharmaceutical formulation of claim 17, wherein the effective amount of the one more compounds is effective to induce apoptosis of cancer cells in a subject.

19. The pharmaceutical formulation of claim 18, wherein the cancer cells are cancer cells harboring WT-p53, and therein the effective amount of the one more compounds is effective to downregulate MDM2 and upregulate p53 in the cancer cells compared to the cancer cells before treatment as shown by Western blot.

20. The pharmaceutical formulation of claim 18, wherein the cancer cells are leukemia cells harboring WT-p53 (e.g. EU-1 leukemia cells), MCF7 cells, RS4;11 cells, or HeLa cells, or a combination thereof.

21. (canceled)

22. (canceled)

23. A method for treating or ameliorating one or more symptoms associated with a cancer in a subject in need thereof comprising
(i) administering to the subject the pharmaceutical formulation of claim 16, wherein step (i) occurs one or more times,
optionally wherein the cancer is leukemia, such as Acute lymphocytic leukemia (ALL), Acute myelogenous leukemia (AML), Chronic lymphocytic leukemia (CLL), or Chronic myelogenous leukemia (CML).

24. -32. (canceled)

33. A method for treating cancer cells in a subject in need thereof comprising (i) administering to the subject the pharmaceutical formulation of claim 16, wherein step (i) occurs one or more times,
optionally wherein the cancer cells are leukemia cells harboring WT-p53, MCF7 cells, RS4;11 cells, or HeLa cells, or a combination thereof.

34. -40. (canceled)
